SEQUENCE LISTING (90 sequences)

SEQ ID NO: 1 - protein, 133 aa, synthetic construct
  source 1..133: note = QP009(SIRPalpha)
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSDYKDDDDKHHHHHH

SEQ ID NO: 2 - protein, 119 aa, synthetic construct
  source 1..119: note = 71C10 heavy chain variable
QVQLQQPGTELVRPGASVKLSCKASGYTFTNYWINWVKQRPGQGLEWIAMIDPSDSETHYNQIFKDKATLTVDKSSNTAYMQLSSLTSGDSAVYYCAMDYGSLYAMDYWGRGTSVTVSS

SEQ ID NO: 3 - protein, 5 aa, synthetic construct
  source 1..5: note = 71C10 HCDR1
NYWIN

SEQ ID NO: 4 - protein, 17 aa, synthetic construct
  source 1..17: note = 71C10 HCDR2
MIDPSDSETHYNQIFKD

SEQ ID NO: 5 - protein, 10 aa, synthetic construct
  source 1..10: note = 71C10 HCDR3
DYGSLYAMDY

SEQ ID NO: 6 - protein, 110 aa, synthetic construct
  source 1..110: note = 71C10 light chain variable region
DIVLTQSPASLAVSLGQRATISCRASKSVSSSGYNYIFWYQQKPGQPPKLLIYLASNLDSGVPARFSGSGSGTDFTLNIHPVEEEDAATYYCQHSRELPTFGGGTKLEIK

SEQ ID NO: 7 - protein, 15 aa, synthetic construct
  source 1..15: note = 71C10 LCDR1
RASKSVSSSGYNYIF

SEQ ID NO: 8 - protein, 7 aa, synthetic construct
  source 1..7: note = 71C10 LCDR2
LASNLDS

SEQ ID NO: 9 - protein, 8 aa, synthetic construct
  source 1..8: note = 71C10 LCDR3
QHSRELPT

SEQ ID NO: 10 - protein, 237 aa, synthetic construct
  source 1..237: note = QD026 light chain
MDMRVPAQLLGLLLLWFPGSRCQIVLTQSPAIMSASPGEKVTLTCSASSSVSSSYLYWYQQKPGSSPKLWIYSTSNLASGVPARFSGSGSGTSYSLTISSMEAEDAASYFCHQWSSYPRTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 462 aa, synthetic construct
  source 1..462: note = QD027 heavy chain
MEFGLSWLFLVAILKGVQCEVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 459 aa, synthetic construct
  source 1..459: note = QD249 heavy chain
MEFGLSWLFLVAILKGVQCEVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 217 aa, synthetic construct
  source 1..217: note = QD163 light chain
DIVLTQSPASLAVSLGQRATISCRASKSVSSSGYNYIFWYQQKPGQPPKLLIYLASNLDSGVPARFSGSGSGTDFTLNIHPVEEEDAATYYCQHSRELPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, synthetic construct
  source 1..449: note = QD164 heavy chain
QVQLQQPGTELVRPGASVKLSCKASGYTFTNYWINWVKQRPGQGLEWIAMIDPSDSETHYNQIFKDKATLTVDKSSNTAYMQLSSLTSGDSAVYYCAMDYGSLYAMDYWGRGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 465 aa, synthetic construct
  source 1..465: note = QD245 heavy chain
MEFGLSWLFLVAILKGVQCQVQLQQPGTELVRPGASVKLSCKASGYTFTNYWINWVKQRPGQGLEWIAMIDPSDSETHYNQIFKDKATLTVDKSSNTAYMQLSSLTSGDSAVYYCAMDYGSLYAMDYWGRGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 110 aa, synthetic construct
  source 1..110: note = QD253 light chain variable region
DIVLTQSPDSLAVSLGERATINCRASKSVSSSGYNYIFWYQQKPGQPPKLLIYLASNLDSGVPDRFSGSGSGTDFTLTISSLQAEDVAVYYCQHSRELPTFGQGTKLEIK

SEQ ID NO: 17 - protein, 119 aa, synthetic construct
  source 1..119: note = QD256 heavy chain variable region
QVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSS

SEQ ID NO: 18 - protein, 110 aa, synthetic construct
  source 1..110: note = QD279 light chain variable region
DIVLTQSPDSLAVSLGERATINCRASQSVRSSGYNWIFWYQQKPGQPPKLLIYLASNRDSGVPDRFSGSGSGTDFTLTISSLQAEDVAVYYCQHSRELPTFGQGTKLEIK

SEQ ID NO: 19 - protein, 110 aa, synthetic construct
  source 1..110: note = QD291 light chain variable region
DIVLTQSPDSLAVSLGERATINCRASKSVGSSGYNWLFWYQQKPGQPPKLLIYLASNRDSGVPDRFSGSGSGTDFTLTISSLQAEDVAVYYCQHSRELPTFGQGTKLEIK

SEQ ID NO: 20 - protein, 110 aa, synthetic construct
  source 1..110: note = QD1581 light chain variable region
DIVLTQSPDSLAVSLGERATINCRASKSVSSSGYNYIFWYQQKPGQPPKLLIYLASNLDPGVPDRFSGSGSGTDFTLTISSLQAEDVAVYYCQHSRELPTFGQGTKLEIK

SEQ ID NO: 21 - protein, 110 aa, synthetic construct
  source 1..110: note = QD1586 light chain variable region
DIVLTQSPDSLAVSLGERATINCRASKSVSSSGYNYIFWYQQKPGQPPKLLIYLASNLDSGVPDRFSGSGSGTDFTLTISSLQAEDVAVYYCQESRELPTFGQGTKLEIK

SEQ ID NO: 22 - protein, 110 aa, synthetic construct
  source 1..110: note = QD1589 light chain variable region
DIVLTQSPDSLAVSLGERATINCRASKSVSSSGYNYIFWYQQKPGQPPKLLIYLASNLDSGVPDRFSGSGSGTDFTLTISSLQAEDVAVYYCQESWELPTFGQGTKLEIK

SEQ ID NO: 23 - protein, 110 aa, synthetic construct
  source 1..110: note = QD1594 light chain variable region
DIVLTQSPDSLAVSLGERATINCRASKSVSSSGYNYIFWYQQKPGQPPKLLIYLASNLDSGVPDRFSGSGSGTDFTLTISSLQAEDVAVYYCQHSRDLPTFGQGTKLEIK

SEQ ID NO: 24 - protein, 110 aa, synthetic construct
  source 1..110: note = QD1770 light chain variable region
DIVLTQSPDSLAVSLGERATINCRASQSVRSSGYNWIFWYQQKPGQPPKLLIYLASNRDSGVPDRFSGSGSGTDFTLTISSLQAEDVAVYYCQESRELPTFGQGTKLEIK

SEQ ID NO: 25 - protein, 110 aa, synthetic construct
  source 1..110: note = QD1771 light chain variable region
DIVLTQSPDSLAVSLGERATINCRASKSVSSSGYNWIFWYQQKPGQPPKLLIYLASNRDSGVPDRFSGSGSGTDFTLTISSLQAEDVAVYYCQESRELPTFGQGTKLEIK

SEQ ID NO: 26 - protein, 449 aa, synthetic construct
  source 1..449: note = QD3270 heavy chain
QVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISAAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 449 aa, synthetic construct
  source 1..449: note = QD3225 heavy chain
QVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 446 aa, synthetic construct
  source 1..446: note = QD3226 heavy chain
QVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 445 aa, synthetic construct
  source 1..445: note = QD3255 heavy chain
QVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 449 aa, synthetic construct
  source 1..449: note = QD3260 heavy chain
QVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 449 aa, synthetic construct
  source 1..449: note = QD3265 heavy chain
QVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 443 aa, synthetic construct
  source 1..443: note = QD3268 heavy chain
EVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISAAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 449 aa, synthetic construct
  source 1..449: note = QD3271 heavy chain
QVQLQQSGPELVKPGASVRISCKASGFTFTNYYIHWVKQRPGQGLEWIGWIYPGNVNTKYNEKFKAKATLTADKSSTTAYMQLSSLASEDSAVYFCARPTIIATDFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISAAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 449 aa, synthetic construct
  source 1..449: note = QD3222 heavy chain
QVQLQQPGTELVRPGASVKLSCKASGYTFTNYWINWVKQRPGQGLEWIAMIDPSDSETHYNQIFKDKATLTVDKSSNTAYMQLSSLTSGDSAVYYCAMDYGSLYAMDYWGRGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 440 aa, synthetic construct
  source 1..440: note = QD3221 heavy chain
EVQLQQSGAELVKPGASVKLSCTASGFNIKDYYIHWVQQRTEQGLEWIGRIDPEDGETKYAPKFQDKATITADTSSNTAYLHLSSLTSEDTAVYYCARWGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 236 aa, synthetic construct
  source 1..236: note = QD3240 light chain
MDMRVPAQLLGLLLLWFPGSRCDIVMTQSHKFMSTSVGDRVNITCKASQGVGTAVGWYQQKPGQSPRLLIYWASTRHTGVPDRFTGSGSGTDFSLAISNVQSEDLADYFCQQYSTYPFTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 15 aa, synthetic construct
  source 1..15: note = QD279/QD1770 VLCDR1
RASQSVRSSGYNWIF

SEQ ID NO: 38 - protein, 7 aa, synthetic construct
  source 1..7: note = QD279/QD291/QD1770/QD1771 VLCDR2
LASNRDS

SEQ ID NO: 39 - protein, 15 aa, synthetic construct
  source 1..15: note = QD291 VLCDR1
RASKSVGSSGYNWLF

SEQ ID NO: 40 - protein, 7 aa, synthetic construct
  source 1..7: note = QD1581 VLCDR2
LASNLDP

SEQ ID NO: 41 - protein, 8 aa, synthetic construct
  source 1..8: note = QD1586/QD1770/QD1771 VLCDR3
QESRELPT

SEQ ID NO: 42 - protein, 8 aa, synthetic construct
  source 1..8: note = QD1589 VLCDR3
QESWELPT

SEQ ID NO: 43 - protein, 8 aa, synthetic construct
  source 1..8: note = QD1594 VLCDR3
QHSRDLPT

SEQ ID NO: 44 - protein, 15 aa, synthetic construct
  source 1..15: note = QD1771 VLCDR1
RASKSVSSSGYNWIF

SEQ ID NO: 45 - protein, 376 aa, synthetic construct
  source 1..376: note = QP094
MEFGLSWLFLVAILKGVQCEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERGSSDYKDDDDKHHHHHH

SEQ ID NO: 46 - protein, 375 aa, synthetic construct
  source 1..375: note = QP096
MEFGLSWLFLVAILKGVQCEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERGSSDYKDDDDKHHHHHH

SEQ ID NO: 47 - protein, 375 aa, synthetic construct
  source 1..375: note = QP098
MEFGLSWLFLVAILKGVQCEEELQVIQPEKSVSVAAGDSATLNCTVSSLIPVGPIQWFRGAGPGRELIYNLKEGHFPRVTAVSDPTKRNNMDFSIRISNITPADAGTYYCVKFRKGSPDVELKSGAGTELSVRAKPSAPVVSGPAVRATAEHTVSFTCESHGFSPRDITLKWFKNGNELSDVQTNVDPAGKSVSYSIRSTARVLLTRRDVHSQVICEVAHVTLQGDPLRGTANLSEAIRVPPFLEVTQQSMRADNQVNVTCQVTKFYPQRLQLTWLENGNVSRTEMASALPENKDGTYNWTSWLLVNVSAHRDDVKLTCQVEHDGQPAVNKSFSVKVSAHPKEQGSNTAAENTGTNERGSSDYKDDDDKHHHHHH

SEQ ID NO: 48 - protein, 375 aa, synthetic construct
  source 1..375: note = QP100
MEFGLSWLFLVAILKGVQCEEELQVIQPEKSVSVAAGESATLNCTATSLIPVGPIQWFRGVGPGRELIYHQKEGHFPRVTPVSDPTKRNNMDFSIRISNITPADAGTYYCVKFRKGSPDVELKSGAGTELSVRAKPSAPVVSGPAVRATAEHTVSFTCESHGFSPRDITLKWFKNGNELSDFQTNVDPAGKSVSYSIRSTARVVLTRRDVHSQVICEVAHVTLQGDPLRGTANLSEAIRVPPFLEFTQQSMRADNQVNVTCQVMKFYPQRLQLTWLENGNVSRTEMASALPENKDGTYNWTSWLLVNVSAHRDDVKLTCQVEHDGQPAVNKSFSVKVSAHPKEQGSNTAAENTGTNERGSSDYKDDDDKHHHHHH

SEQ ID NO: 49 - protein, 372 aa, synthetic construct
  source 1..372: note = QP271
MEFGLSWLFLVAILKGVQCEEELQVIQPEKSVSVAAGDSATLNCTVTSLIPVGPIQWFRGAGPGRELIYHQKEGHFPRVTSVSESTKRNNMDFSIHISNITPADAGTYYCVKFRKGSPDVEVKSGAGTELSVRAKPSAPVVSGPAVRATAEHTVSFTCESHGFSPRDITLKWFKNGNELSDFQTNVDPAGKSVSYSIRSTARVVLTRRDVHSQVICEVAHVTLQGDPLRGTANLSEAIRVPPFLEVTQQSMRADNQVNVTCQVTKFYPQRLQLTWLENGNVSRTEMASALPENKDGTYNWTSWLLVNVSAHRDDVKLTCQVEHDGQPAVNKSFSVKVSAHPKEQGSNTAAENTGTNERDYKDDDDKHHHHHH

SEQ ID NO: 50 - protein, 372 aa, synthetic construct
  source 1..372: note = QP273
MEFGLSWLFLVAILKGVQCEEELQVIQPEKSVSVAAGESATLNCTATSLIPVGPIQWFRGVGPGRELIYSQKEGHFPRVTPVSDPTKRNNMDFSIRISNITPADAGTYYCVKFRKGSPDVELKSGAGTELSVRAKPSAPVVSGPAVRATAEHTVSFTCESHGFSPRDITLKWFKNGNELSDFQTNVDPAGKSVSYSIRSTARVVLTRRDVHSQVICEVAHVTLQGDPLRGTANLSEAIRVPPFLEVTQQSMRADNQVNVTCQVTKFYPQRLQLTWLENGNVSRTEMASALPENKDGTYNWTSWLLVNVSAHRDDVKLTCQVEHDGQPAVNKSFSVKVSAHPKEQGSNTAAENTGTNERDYKDDDDKHHHHHH

SEQ ID NO: 51 - protein, 123 aa, synthetic construct
  source 1..123: note = human SIRP alpha V1
GVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 52 - protein, 122 aa, synthetic construct
  source 1..122: note = human SIRP alpha V2
GVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 53 - protein, 122 aa, synthetic construct
  source 1..122: note = human SIRP alpha V3
GVAGEEELQVIQPDKSVSVAAGESAILLCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 54 - protein, 123 aa, synthetic construct
  source 1..123: note = human SIRP alpha V4
GVAGEEGLQVIQPDKSVSVAAGESAILHCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 55 - protein, 123 aa, synthetic construct
  source 1..123: note = human SIRP alpha V5
GVAGEEELQVIQPDKFVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPS

SEQ ID NO: 56 - protein, 123 aa, synthetic construct
  source 1..123: note = human SIRP alpha V6
GVAGEEELQVIQPDKSVLVAAGETATLRCTATSLIPVGPIQWFRGAGPGRELIYNQKEGHFPRVTTVSDLTKRNNMDFPIRIGNITPADAGTYYCVKFRKGSPDDVEFKSGAGTELSVRAKPS

SEQ ID NO: 57 - protein, 122 aa, synthetic construct
  source 1..122: note = human SIRP alpha V7
GVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRGKPS

SEQ ID NO: 58 - protein, 122 aa, synthetic construct
  source 1..122: note = human SIRP alpha V8
GVAGEEELQVIQPDKSVLVAAGETATLRCTATSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 59 - protein, 123 aa, synthetic construct
  source 1..123: note = human SIRP alpha V9
GVAGEEELQVIQPDKSVLVAAGETATLRCTATSLIPVGPIQWFRGAGPGRELIYNQKEGHFPRVTTVSDLTKRNNMDFSIRISNITPADAGTYYCVKFRKGSPDDVEFKSGAGTELSVRAKPS

SEQ ID NO: 60 - protein, 122 aa, synthetic construct
  source 1..122: note = human SIRP alpha V10
RVAGEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 61 - protein, 233 aa, synthetic construct
  source 1..233: note = FC(mouse IgG2a)
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 62 - protein, 129 aa, synthetic construct
  source 1..129: note = QD509 VHH
EVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSS

SEQ ID NO: 63 - protein, 10 aa, synthetic construct
  source 1..10: note = QD509 VHH CDR1
GFTLDDYAIG

SEQ ID NO: 64 - protein, 17 aa, synthetic construct
  source 1..17: note = QD509 VHH CDR2
CISKSGETTFFVESVKD

SEQ ID NO: 65 - protein, 22 aa, synthetic construct
  source 1..22: note = QD509 VHH CDR3
ATGSWCTVGSMSRQFYRQFFHS

SEQ ID NO: 66 - protein, 597 aa, synthetic construct
  source 1..597: note = QD3282
QVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISAAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSS

SEQ ID NO: 67 - protein, 217 aa, synthetic construct
  source 1..217: note = QD279
DIVLTQSPDSLAVSLGERATINCRASQSVRSSGYNWIFWYQQKPGQPPKLLIYLASNRDSGVPDRFSGSGSGTDFTLTISSLQAEDVAVYYCQHSRELPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 351 aa, synthetic construct
  source 1..351: note = QD623
EVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSSGGGGSDIVLTQSPDSLAVSLGERATINCRASQSVRSSGYNWIFWYQQKPGQPPKLLIYLASNRDSGVPDRFSGSGSGTDFTLTISSLQAEDVAVYYCQHSRELPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 582 aa, synthetic construct
  source 1..582: note = QD626
EVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSSGGGGSQVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISAAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 379 aa, synthetic construct
  source 1..379: note = QP3447(Fc-PD-L1 VHH)
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISAAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSS

SEQ ID NO: 71 - protein, 360 aa, synthetic construct
  source 1..360: note = QP509(PD-L1 VHH-Fc)
EVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISAAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 241 aa, synthetic construct
  source 1..241: note = QD250
MDMRVPAQLLGLLLLWFPGSRCDVVMTQTPLSLPVSLGDQASISCRSSQSLVHSYGNTYLYWYLQKPGQSPKLLIYRVSNRFSGVPDRFSGSGSGTDFTLKISRVEAEDLGVYFCFQGTHVPYTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 466 aa, synthetic construct
  source 1..466: note = QD251
MEFGLSWLFLVAILKGVQCQVQLQQPGAELVRPGSSVKLSCKASGYTFTSYWVHWVKQRPIQGLEWIGNIDPSDSDTHYNQKFKDKASLTVDKSSSTAYMQLSSLTFEDSAVYYCVRGGTGTMAWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 469 aa, synthetic construct
  source 1..469: note = QD3750
MEFGLSWLFLVAILKGVQCQVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 236 aa, synthetic construct
  source 1..236: note = QD3751
MDMRVPAQLLGLLLLWFPGSRCDIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 214 aa, synthetic construct
  source 1..214: note = QD1181
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 448 aa, synthetic construct
  source 1..448: note = QD1180
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 374 aa, synthetic construct
  source 1..374: note = QP001
MEFGLSWLFLVAILKGVQCQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNEEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 370 aa, synthetic construct
  source 1..370: note = QP002
MEFGLSWLFLVAILKGVQCEEELQVIQPDKSVLVAAGETATLRCTVTSLIPVGPIQWFRGAGPGRELIYNQKEGHFPRVTTVSDSTKRNNMDFSIRIGNITPADAGTYYCVKFRKGSPDDVEFKSGAGTELSVRAKPSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 592 aa, synthetic construct
  source 1..592: note = QP093
MEFGLSWLFLVAILKGVQCEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNEREPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 81 - protein, 591 aa, synthetic construct
  source 1..591: note = QP095
MEFGLSWLFLVAILKGVQCEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNEREPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 82 - protein, 356 aa, synthetic construct
  source 1..356: note = QD624
EVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSSGGGGSGGGGSDIVLTQSPDSLAVSLGERATINCRASQSVRSSGYNWIFWYQQKPGQPPKLLIYLASNRDSGVPDRFSGSGSGTDFTLTISSLQAEDVAVYYCQHSRELPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 361 aa, synthetic construct
  source 1..361: note = QD625
EVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSSGGGGSGGGGSGGGGSDIVLTQSPDSLAVSLGERATINCRASQSVRSSGYNWIFWYQQKPGQPPKLLIYLASNRDSGVPDRFSGSGSGTDFTLTISSLQAEDVAVYYCQHSRELPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 587 aa, synthetic construct
  source 1..587: note = QD627
EVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSSGGGGSGGGGSQVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISAAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 592 aa, synthetic construct
  source 1..592: note = QD628
EVQLLESGGGLVQPGGSLRLSCAASGFTLDDYAIGWFRQAPGKGREGVSCISKSGETTFFVESVKDRFTISRDNSKNTLYLQMNSLRAEDTAVYYCATGSWCTVGSMSRQFYRQFFHSWGQGTLVTVSSGGGGSGGGGSGGGGSQVQLVQSGAEVKKPGASVKVSCKASGYTFTNYWINWVRQAPGQGLEWIAMIDPSDSETHYNQIFKDRATLTTDTSTSTAYMELRSLRSDDTAVYYCAMDYGSLYAMDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISAAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 454 aa, synthetic construct
  source 1..454: note = mFC(mIgG2a)
APVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNEREPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 87 - protein, 5 aa, synthetic construct
  source 1..5: note = linker
GSGGS

SEQ ID NO: 88 - protein, 4 aa, synthetic construct
  source 1..4: note = linker
GGGS

SEQ ID NO: 89 - protein, 5 aa, synthetic construct
  source 1..5: note = linker
GGGGS

SEQ ID NO: 90 - protein, 323 aa, synthetic construct
  source 1..323: note = Fc(IgG2m 4)
ASTKGPSVFPLAPCSRSTSESTAALGCLVKDYFPEPVTVSWNSGALTSGVHTFPAVLQSSGLYSLSSWTVPSSNFGTQTYTCNVDHKPSNTKVDKTVERKCCVECPPCPAPPVAGPSVFLFPPKPKDTLMISRTPEVTCWVDVSQEDPEVQFNWYVDGVEVHNAKTKPREEQFNSTYRVVSVLTVLHQDWLNGKEYKCKVSNKGLPSSIEKTISKAKGQPREPQWTLPPSQEEMTKNQVSLTCLVKGFYPSDIAVEWESNGQPENNYKTTPPVLDSDGSFFLYSRLTVDKSRWOEGNVFSCSVMHEALHNHYTOKSLSLSLGK

(19) United States
(12) Patent Application Publication
(10) Pub. No.: US 2025/0326840 A1
(43) Pub. Date: Oct. 23, 2025

(54) BISPECIFIC ANTIBODY TARGETING SIRP-alpha AND PD-L1 OR ANTIGEN-BINDING FRAGMENT THEREOF AND USE

(71) Applicant: QURE BIOTECHNOLOGY (SHANGHAI) CO., LTD., Shanghai (CN)

(72) Inventors: Xiangdong QU, Shanghai (CN); Qin PAN, Shanghai (CN); Yejie DU, Shanghai (CN)

(21) Appl. No.: 18/870,605

(22) PCT Filed: May 6, 2023
(86) PCT No.: PCT/CN2023/092580

§ 371 Date: Nov. 29, 2024

(30) Foreign Application Priority Data
May 28, 2022 (CN) 202210595262.1

(51) Int. Cl. C07K 16/28; A61K 39/00; A61K 39/395; A61P 35/00

(52) U.S. Cl. CPC C07K 16/2803; A61K 39/3955; A61P 35/00; C07K 16/2827; A61K 2039/507; C07K 2317/24; C07K 2317/31; C07K 2317/569; C07K 2317/622; C07K 2317/73; C07K 2317/76; C07K 2317/92

(57) ABSTRACT

A bispecific antibody targeting SIRPα and PD-L1 or an antigen-binding fragment thereof and a use. The bispecific antibody comprises an SIRPα binding domain and a PD-L1 binding domain; the SIRPα binding domain comprises a heavy chain variable region and a light chain variable region, and the PD-L1 binding domain comprises: a VHH fragment. Also provided are a drug comprising the bispecific antibody targeting SIRPα and PD-L1 or the antigen-binding fragment thereof, a nucleic acid molecule, a vector, a host cell obtained by conversion of the vector, and a pharmaceutical use of the antibody.

### TECHNICAL FIELD

The present invention relates to the field of biomedical technology, specifically to a bispecific antibody targeting SIRPα and PD-L1 or antigen binding fragment thereof, and use therefor.

### BACKGROUND

PD-1 (CD279) was first reported in 1992. The human PD-1 coding gene PDCD1 is located at 2q37.3, with a total length of 2097 bp and composed of six exons. PD-1 is a membrane protein belonging to the CD28 immunoglobulin superfamily. It is mainly expressed on the surface of activated T cells. In addition, it is also expressed in low abundance on CD4-CD8-T cells, activated NK cells, and monocytes in the thymus. PD-1 has two ligands, namely PD-L1 (CD274, B7-H1) and PD-L2 (CD273, B7-DC) of the B7 protein family. The amino acid sequences of PD-L1 and PD-L2 are 40% identical. The main difference between the two ligands lies in their different expression patterns. PD-L1 is constitutively low expressed in APCs, non hematopoietic cells (such as vascular endothelial cells and pancreatic islet cells), and immune exempt sites (such as placenta, testes, and eyes). Inflammatory cytokines such as type I and type II interferons, TNF-α, and VEGF can induce the expression of PD-L1. PD-L2 is only expressed in activated macrophages and dendritic cells. After PD-1 binds to PD-L1 on activated T cells, the ITSM motif of PD-1 undergoes tyrosine phosphorylation, leading to the dephosphorylation of downstream protein kinases Syk and PI3K, inhibiting the activation of downstream pathways such as AKT and ERK, ultimately inhibiting the transcription and translation of genes and cytokines required for T cell activation, and exerting a negative regulatory effect on T cell activity. In tumor cells, tumor cells and tumor microenvironment negatively regulate T cell activity and suppress immune responses by upregulating PD-L1 expression and binding to PD-1 on the surface of tumor specific CD8+T cells. There is increasing evidence to suggest that tumors utilize PD-1-dependent immune suppression for immune evasion. High expression of PD-L1 and PD-L2 has been found in various solid tumors and hematological malignancies. In addition, there is a strong correlation between the expression of PD-Ls and the poor prognosis of tumor cells.

The phagocytic activity of tumor associated macrophages (TAMs) in the tumor microenvironment is inhibited due to the high expression of CD47 protein on the surface of almost all tumor cells, which can bind to the signal regulatory protein α (SIRPα) on the surface of bone marrow cells and emit a “don't eat me” or “self” signal to the body, thereby inhibiting phagocytic activity. CD47, also known as integrin associated protein (IAP), is a widely expressed transmembrane glycoprotein, and belongs to the immunoglobulin (Ig) superfamily. CD47 has a molecular weight of 50 kD, and its structure contains a large number of glycosylated N-terminal IgV variable domains, five highly hydrophobic transmembrane domains, and a short C-terminal cytoplasmic tail region. The four selective splicing forms of the C-terminal cytoplasmic tail region determine the expression of CD47 in different tissues. The corresponding SIRPα, also known as SHPS-1, BIT, or CD172a protein, is a transmembrane protein mainly expressed on myeloid cells, including macrophages, bone marrow dendritic cells, granulocytes, mast cells, and their precursor cells. SIRPα consists of three extracellular Ig like domains and four intracellular tyrosine residues, which are speculated to be phosphorylation sites. After phosphorylation, SIRPα activates downstream signaling pathways by binding to the SH2 domain of SHP-1/2 protein and activating it. The expression of SHP-1 and SHP-2 proteins is tissue-specific, therefore SIRPα is a docking protein that recruits and activates downstream protein phosphatases in response to extracellular stimuli. Oldenborg first reported that mature red blood cells (RBCs) protect themselves from clearance by binding to splenic macrophage SIRPα through CD47.

Subsequently, researchers found that RBCs can also bind to monocyte SIRPα to inhibit Fcγ receptor dependent phagocytosis, which is achieved by dephosphorylating the key molecule myosin IIA in phagocytosis. In clinical practice, CD47 overexpression has been found in a variety of solid tumors and hematological malignancies, including acute myeloid leukemia (AML), acute lymphoid leukemia (ALL), chronic myeloid leukemia (CMIL), non-Hodgkin's lymphoma (NHL), breast cancer, bladder cancer, ovarian cancer, colon cancer, etc. The essence is that tumor cells escape the cell clearance effect of macrophages through the above-mentioned regulatory mechanism. CD47 also affects other biological processes by binding to other receptors or through signal transduction in its intracellular cytoplasmic region. The interaction between CD47 and thrombospondin-1 (TSP-1) or vascular endothelial growth factor receptor 2 (VEGFR-2) inhibits angiogenesis, thereby limiting tumor growth.

The biological function of CD47 itself determines that CD47 therapeutic antibodies and SIRPα-Fc recombinant protein may have hematological toxicity or the risk of anemia, which has been reported in CD47 gene knockout NOD mice and mouse models treated with CD47 antibodies. In addition, endothelial cell CD47 has been reported to promote transendothelial migration of T cells by interacting with SIRPγ through cell adhesion, and SIRPγ is mainly expressed in T cells rather than bone marrow cells. Therefore, using SIRPα antibodies is a more optimal choice for blocking the CD47-SIRPα signaling pathway. In addition, Weissman research group at Stanford University has demonstrated that the humanized SIRPα antibody KWAR23, which was screened by them, could effectively inhibit the growth of Burkitt lymphoma in human SIRPα gene knock-in SRG mice (Rag2−/− Il2r γ−/−) in combination with rituximab, but KWAR23 alone had no significant therapeutic effect.

### SUMMARY OF THE INVENTION

The first objective of the present invention is to provide a bispecific antibody targeting SIRPα and PD-L1 or antigen binding fragment thereof. The bispecific antibody targeting SIRPα and PD-L1 or antigen binding fragment thereof provided by the present invention comprises: a SIRPα binding domain and a PD-L1 binding domain; wherein, the SIRPα binding domain comprises: a heavy chain variable region and a light chain variable region; The heavy chain variable region comprises: VHCDR1, VHCDR2, and VHCDR3 with amino acid sequences as shown in SEQ ID NOs: 3, 4, and 5, respectively; The light chain variable region comprises: VLCDR1, VLCDR2, and VLCDR3 with amino acid sequences as shown in SEQ ID NOs: 37, 38, and 9, respectively; The PD-L1 binding domain comprises: a VHH fragment, which comprises CDR1, CDR2, and CDR3 with amino acid sequences as shown in SEQ ID NOs: 63, 64, and 65, respectively.

Optionally, the sequence of the heavy chain variable region of the SIRPα binding domain is as shown in SEQ ID NO: 17 or has at least 85% sequence identity with SEQ ID NO: 17; Alternatively, the sequence of the light chain variable region of the SIRPα binding domain is selected from SEQ ID NO: 18 or has at least 85% sequence identity with SEQ ID NO: 18.

Optionally, the sequence of the VHH fragment is as shown in SEQ ID NO: 62 or has at least 85% sequence identity with SEQ ID NO: 62.

Optionally, the bispecific antibody or antigen binding fragment thereof further comprises: a heavy chain constant region selected from human-derived IgG1, IgG2, IgG3, or IgG4 or variants thereof, and a light chain constant region selected from human-derived κ, λ chains or variants thereof.

Optionally, the heavy chain constant region comprises: an Fc fragment or variants thereof; The variant of the Fc fragment is derived from IgG1, according to EU Numbering, including mutation sites: L234A, L235A, and K338A.

Optionally, the bispecific antibody or antigen binding fragment thereof comprises: a first polypeptide chain and a second polypeptide chain; The first polypeptide chain comprises: the heavy chain variable region of the SIRPα binding domain, the heavy chain constant region, and the VHH fragment; The VHH fragment is fused with the N-terminus of the heavy chain variable region of the SIRPα binding domain, or the VHH fragment is fused with the C-terminus of the heavy chain constant region; The second polypeptide chain comprises: a light chain variable region of the SIRPα binding domain and a light chain constant region.

Optionally, the bispecific antibody or antigen binding fragment thereof comprises: a first polypeptide chain and a second polypeptide chain; The first polypeptide chain comprises: the heavy chain variable region of the SIRPα binding domain and the heavy chain constant region; The second polypeptide chain comprises: the light chain variable region of the SIRPα binding domain, the light chain constant region, and the VHH fragment; The VHH fragment is fused with the N-terminus of the light chain variable region of the SIRPα binding domain.

Optionally, the bispecific antibody or antigen binding fragment thereof is a symmetrical structure comprising two first polypeptide chains and two second polypeptide chains.

Optionally, the bispecific antibody or antigen binding fragment thereof further comprises: a linking sequence; The linking sequence may be selected from (GGGGS)n, wherein n is an integer from 1 to 4.

Optionally, the amino acid sequence of the first polypeptide chain is as shown in any one of SEQ ID NOs: 66, 26, 69, 84, 85; Alternatively, the amino acid sequence of the second polypeptide chain is as shown in any one of SEQ ID NOs: 67, 68, 82, 83.

Optionally, the amino acid sequence of the first polypeptide chain is as shown in SEQ ID NO: 66, and the amino acid sequence of the second polypeptide chain is as shown in SEQ ID NO: 67.

The second objective of the present invention is to provide a drug comprising the aforementioned bispecific antibody targeting SIRPα and PD-L1 or antigen binding fragment thereof.

Optionally, the drug further comprises one or more other cancer therapeutic agents.

The third objective of the present invention is to provide a nucleic acid molecule encoding the aforementioned bispecific antibody targeting SIRPα and PD-L1 or antigen binding fragment thereof.

The fourth objective of the present invention is to provide a vector comprising the aforementioned nucleic acid molecule.

The fifth objective of the present invention is to provide a host cell transformed with the aforementioned vector.

The sixth objective of the present invention is to provide a use of the aforementioned bispecific antibody targeting SIRPα and PD-L1 or antigen binding fragment thereof in the manufacture of a medicament for inhibiting or treating a disease, disorder or condition.

Optionally, the disease, disorder or condition includes: cancer, solid tumor, chronic infection, inflammatory disease, multiple sclerosis, autoimmune disease, neurological disease, brain injury, nerve injury, polycythemia, hemochromatosis, trauma, septic shock, fibrosis, atherosclerosis, obesity, type II diabetes, allograft dysfunction or arthritis.

Optionally, the cancer is selected from anal cancer, appendiceal cancer, astrocytoma, basal cell cancer, gallbladder cancer, gastric cancer, lung cancer, bronchial cancer, bone cancer, hepatobiliary cancer, pancreatic cancer, breast cancer, liver cancer, ovarian cancer, testicular cancer, renal cancer, renal pelvis and ureter cancer, salivary gland cancer, small intestine cancer, urethra cancer, bladder cancer, head and neck cancer, spinal cancer, brain cancer, cervical cancer, uterine cancer, endometrial cancer, colon cancer, colorectal cancer, rectal cancer, esophageal cancer, gastrointestinal cancer, skin cancer, prostate cancer, pituitary cancer, vaginal cancer, thyroid cancer, laryngeal cancer, glioblastoma, melanoma, myelodysplastic syndrome, sarcoma, teratoma, chronic lymphoblastic leukemia (CLL), chronic myeloid leukemia (CMIL), acute lymphoblastic leukemia (ALL), acute myeloid leukemia (ANIL), Hodgkin's lymphoma, non-Hodgkin's lymphoma, multiple myeloma, T or B-cell lymphoma, gastrointestinal stromal tumor, soft tissue tumor, hepatocellular carcinoma or adenocarcinoma.

Optionally, the medicament is used in combination with one or more other drugs.

Optionally, the other drugs include rituximab.

Compared with the prior art, the present invention has the following beneficial effects:
(1) The bispecific antibody provided by the present invention can simultaneously target SIRPα and PD-L1, mediating immune cell killing while targeting tumor cells; Targeting SIRPα can avoid the risk of hematological toxicity or inducing anemia.
(2) The SIRPα binding domain sequence of the bispecific antibody of the present invention is novel.
(3) The bispecific antibody provided by the present invention has a unique configuration, which can efficiently target the target protein and achieve efficient tumor killing effect.
(4) The bispecific antibody provided by the present invention can bind to all subtypes of human SIRPα protein, which is beneficial for clinical development.

### DESCRIPTION OF THE DRAWINGS

FIGS. 1 to 6 show the results of Binding-ELISA detection.

FIG. 7 shows the result of Blocking-ELISA detection.

FIG. 8 shows the results of FACS detection of SIRPα antibody binding to human renal clear cell adenocarcinoma cells 786-O naturally expressing human SIRPα.

FIGS. 9 to 11 show the ADCP results of in vitro functional assay of anti-SIRPα antibodies.

FIG. 12 shows the tumor growth curve reflected by the tumor imaging signal value in each group and the D18 imaging signal intensity result.

FIG. 13 shows the survival curves of each group.

FIGS. 14 to 23 show the results of ELISA detection of the binding of the antibody CHO71 of the present invention and the control antibodies 18D5 and KWAR23 to SIRPα V1/V2/V3/V4/V5/V6/V7/V8/V9/V10 subtypes.

FIG. 24 shows the amino acid sequence alignment of known human SIRP alpha binding domain alleles.

FIG. 25 shows the binding curve of antibody molecules to human PD-L1 protein detected by ELISA.

FIG. 26 shows the binding curve of antibody molecules to human SIRPα V1 protein detected by ELISA.

FIG. 27 shows the binding curve of antibody molecules to human SIRPα V2 protein detected by ELISA.

FIG. 28 shows the curve of PD-L1 binding to PD-1 blocked by antibody molecules of ELISA detection.

FIG. 29 shows the curve of CD47 binding to SIRPα blocked by antibody molecules of ELISA detection.

FIG. 30 shows the results of FACS detection of antibody molecules synergistically enhancing CD20 antibody Rituxan dependent ADCP.

FIG. 31 shows the results of IL-2 secretion after 48 hours of SEB stimulated PBMC proliferation by antibody molecules.

FIGS. 32 to 40 shows the binding curve of antibody molecules such as Q-1801 to human SIRPα V1/V2/V3/V4/V5/V6/V7/V8/V9 subtypes detected by ELISA.

FIG. 41 shows the binding curve of antibody molecules such as Q-1801 to human SIRPα V1 detected by FACS.

FIG. 42 shows the binding curve of antibody molecules such as Q-1801 to human SIRPα V2 detected by FACS.

FIG. 43 shows the binding curve of antibody molecules such as Q-1801 to human PD-L1 protein detected by ELISA.

FIG. 44 shows the binding curve of antibody molecules such as Q-1801 to human PD-L1 detected by FACS.

FIG. 45 shows the curve of SIRPα/CD47 binding blocked by antibody molecules such as Q-1801 of ELISA detection.

FIG. 46 shows the curve of PD-1/PD-L1 binding blocked by antibody molecules such as Q-1801 of ELISA detection.

FIG. 47 shows the curve of PD-1/CD80 binding blocked by antibody molecules such as Q-1801 of ELISA detection.

FIG. 48 shows the results of Q-1801 synergistically enhancing CD20 antibody Rituxan dependent ADCP.

FIG. 49 shows the results of Q-1801 synergistically enhancing CD20 antibody Rituxan dependent ADCP.

FIG. 50 shows the result of IL-2 secretion in the supernatant of mixed lymphocyte reaction for 48 hours.

FIG. 51 shows the result of IFN-γ secretion in the supernatant of mixed lymphocyte reaction for 120 hours.

FIG. 52 shows the results of IL-2 secretion after 48 hours of SEB stimulated PBMC proliferation by antibody molecules such as Q-1801.

FIG. 53 shows the results of IFN-γ secretion after 120 hours of SEB stimulated PBMC proliferation by antibody molecules such as Q-1801.

FIG. 54 shows the trend of tumor growth after administration.

FIG. 55 shows the trend of weight changes in animals after administration.

FIG. 56 shows the live imaging photos of mice on day 0 after grouping.

FIG. 57 shows the live imaging photos of mice on day 7 after grouping.

FIG. 58 shows the live imaging photos of mice on day 14 after grouping.

FIG. 59 shows the trend of tumor volume changes in the MC38-hPD-L1 colon cancer tumor model after administration.

FIG. 60 shows the tumor growth curves of individual mice in different groups of MC38-hPD-L1 colon cancer tumor model.

FIG. 61 shows the weight change curves of mice in each group of MC38-hPD-L1 colon cancer tumor model after administration.

FIG. 62 shows the trend of tumor volume changes in the CT26-hPD-L1&hSIRPα colon cancer tumor model after administration.

FIG. 63 shows the tumor growth curves of each mice of CT26-hPD-L1&hSIRPα colon cancer tumor model after administration.

FIG. 64 shows the weight change curves of mice in each group of CT26-hPD-L1&hSirpα colon cancer tumor model after administration.

FIG. 65 shows the re-inoculated trend of tumor volume changes in mice with tumor regression after the first administration.

FIG. 66 shows the tumor growth curves of each mice re-inoculated with CT26-hPD-L1&hSIRPα colon tumor.

FIG. 67 shows the weight change curves of mice in each group of CT26-hPD-L1&hSIRPα colon tumor re-inoculation.

FIG. 68 shows the changes in tumor volume of NCG mice inoculated subcutaneously with non-small cell lung cancer HCC827 model in different administration groups.

FIG. 69 shows the weight changes of mice in different groups.

### DETAILED DESCRIPTION

### Term

An “antibody (Ab)” refers to an immunoglobulin molecule (Ig) that comprises at least one antigen-binding site and can specifically bind to an antigen.

An “antigen” refers to a substance that can induce an immune response and specifically bind to antibody in the body. The binding of an antibody to an antigen is mediated by the interaction formed between the two, including hydrogen bonds, Van der Waals' force, ionic bonds, and hydrophobic bonds. The region where the antigen surface binds to the antibody is called the “antigenic determinant cluster” or “epitope”. Generally speaking, each antigen has multiple determinant clusters.

“Fusion” refers to the connection of components through peptide bonds or with the help of one or more peptide linkers. The different components of an antibody molecule are connected by “peptide linkers” to ensure correct protein folding and peptide stability. Peptide linkers can be selected as amino acid sequences with low immunogenicity. Herein, “peptide linker” and “linking sequence” have the same meaning. The linking sequence connects the various components of the fusion protein. In a specific embodiment, suitable linking sequences such as (GS) n, (GSGGS (SEQ ID NO: 87)) n, (GGGS (SEQ ID NO: 88) n, (GGGGS (SEQ ID NO: 89)) n can be selected. The n can be chosen from 1-4, or a larger number.

The term “antibody” referred to in the present invention is understood in its widest sense and includes monoclonal antibodies (including full-length monoclonal antibodies), polyclonal antibodies, antibody fragments, and multispecific antibodies containing at least two different antigen-binding domains (e.g., bispecific antibodies). Antibodies also include mouse-derived antibodies, humanized antibodies, chimeric antibodies, human antibodies, and antibodies derived from other origins. The antibodies of the present invention can originate from any animal, including but not limited to immunoglobulin molecules of humans, non-human primates, mice, rats, cows, horses, chickens, camels, alpacas, etc. Antibodies can contain additional changes, such as non-natural amino acids, Fc effector functional mutations, and glycosylation site mutations. Antibodies also include post translational modified antibodies, fusion proteins containing antigenic determinant clusters of antibodies, and any other modified immunoglobulin molecules containing antigenic recognition sites, as long as these antibodies exhibit the desired biological activity.

The basic structure of a conventional antibody is a Y-shaped monomer connected by two identical heavy chains (H) and two identical light chains (L) via disulfide bonds. Each chain is composed of 2-5 structural domains (also known as functional regions) containing approximately 110 amino acids, with similar sequences but different functions. The amino acid sequences near the N-terminus of the light and heavy chains in an antibody molecule undergo significant changes, forming a structural domain called the variable region (V region); and the region near the C-terminus where the amino acid sequences are relatively constant is called the constant region (C region).

The V regions of heavy and light chains are called VH and VL, respectively. VH and VL each have three regions of highly variable amino acid composition and arrangement, known as the hypervariable region (HVR). This region forms a spatial conformation complementary to the antigen epitope, also known as the complementarity determining region (CDR). The three CDRs of VH are represented by VHCDR1, VHCDR2, and VHCDR3, while the three CDRs of VL are represented by VLCDR1, VLCDR2, and VLCDR3, respectively. VH and VL have a total of 6 CDRs that together form the antigen-binding site. The diversity of amino acids in the CDR region is the molecular basis for the specific binding of antibodies to a large number of different antigens. The composition and arrangement order of amino acids outside of CDR in the V region have relatively little change, and are called frame region or framework region (FR). VH and VL each have four framework regions, represented by FR1, FR2, FR3, and FR4, respectively. VH and VL each consist of three CDRs and four FRs, arranged from the amino-terminus (N-terminus) to the carboxyl-terminus (C-terminus) in the following order: FR1, CDR1, FR2, CDR2, FR3, CDR3, FR4.

According to the amino acid sequence of the antibody heavy chain constant region, human immunoglobulins can be divided into 5 classes: IgM, IgG, IgA, IgD, and IgE. It can also be further divided into different subclasses (isotypes), such as human IgG can be divided into IgG1, IgG2, IgG3, IgG4; IgA can be divided into IgA1 and IgA2. No subclass of IgM, IgD, and IgE has been found. Light chains can be classified as κ chain and λ chain according to their amino acid sequences. The antibodies of the present invention can be of any class (such as IgM, IgG, IgA, IgD, IgE) or subclass (such as IgG1, IgG2, IgG3, IgG4, IgA1, IgA2).

The constant regions of heavy and light chains are referred to as CH and CL, respectively. The heavy chain constant region of IgG, IgA, and IgD has three domains of CH1, CH2, and CH3, and the heavy chain constant region of IgM and IgE has four domains of CH1, CH2, CH3, and CH4.

The region located between CH1 and CH2 is hinge region, which is rich in proline, making it easy to stretch and bend. It can change the distance between the two Y-shaped arms, which is conducive to the simultaneous binding of the two arms to epitopes.

An “antigen-binding fragment” refers to a Fab fragment, a F(ab′)2 fragment, a Fv fragment, a ScFv fragment, or the like having antigen-binding activity. A “Fab fragment” (fragment of antigen binding, Fab) refers to an antibody fragment consisting of VL, VH, CL and CH1 domains, and it binds to a single epitope (monovalent). Those skilled in the art know that papain hydrolyzes IgG to form two identical Fab segments and one Fc segment; pepsin hydrolyzes IgG to form one F(ab′)2 segment and several polypeptide fragments (pFc′). If the disulfide bond between F (ab′) 2 heavy chains is broken, two Fab′ fragments can be formed, and the latter can be further enzymatically hydrolyzed into Fv fragments. An Fv fragment contains a heavy chain variable region and a light chain variable region of the antibody, but no constant region. Single chain antibody fragment (scFv), also known as single chain antibody, is formed by linking the heavy chain variable region and light chain variable region of the antibody through a linking fragment (linker).

In 1993, Hamers laboratory discovered that in addition to conventional quadruple antibodies, there were also a large number of molecules similar to immunoglobulin G (IgG) in camel serum. This type of molecule is called heavy chain antibody (HCAb), which naturally lacks the conventional antibody light chain and heavy chain constant region CH1, but still has strong binding affinity to antigens. Hamers laboratory also analyzed and identified the structure and sequence of heavy chain antibodies in camel serum, and found that the antigen binding region of heavy chain antibodies is only composed of variable region fragments, which is equivalent to the functional equivalent of conventional antibody antigen binding fragment (Fab). Therefore, the antigen recognition region fragment of heavy chain antibody is referred to as VHH (variable domain of the heavy chain of heavy-chain antibody), and on this basis, nanobody containing only the VHH domain has been developed. Nanobody is also known as single domain antibody (sdAb).

Nanobodies are easy to be modified and formed multivalent forms. Due to their small molecular weight, nanobodies are encoded by a single gene, making them easy to be genetically engineered. Multiple nanobodies can aggregate through short linking sequences, and can even be linked and combined with conventional antibody Fab fragments, Fv fragments, ScFv fragments, etc., to form multivalent or multi-specific antibody structures. Bivalent or multivalent antibodies can recognize the same epitope, but have a higher antigen affinity than monovalent antibodies. Bispecific or multispecific antibodies can bind to different targets or different binding regions on the same target, and have stronger antigen recognition ability than monovalent antibodies.

Nanobodies can easily form new fusion molecules with other structures such as BSA, IgG Fc, etc. In the new fusion molecule, the nanobody binds to its target antigen in a targeted manner, and the part fused with the nanobody can perform its corresponding function. Therefore, it can be used in combination with other drugs or applied as a diagnostic and experimental research tool in various fields. Nanobody screening can be divided into steps such as alpaca immunization, lymphocyte extraction, nanobody library construction, phage library construction, specific phage screening, E. coli expression, and antibody purification, etc.

The terms “Fc”, “Fc segment” or “Fc fragment” refer to a fragment crystallizable, which has no antigen binding activity and is the interaction site of an antibody with an effector molecule or a cell surface Fc receptor (FcR). The Fc fragment comprises the heavy chain constant region polypeptides of antibody, except for the heavy chain constant region CH1. Fc fragments bind to cells with corresponding Fc receptors on their surface, resulting in different biological effects. In ADCC effect (antibody dependent cell-mediated cytotoxicity), the Fab segment of the antibody binds to the antigen epitopes of virus-infected cells or tumor cells, and its Fc segment binds to the FcR on the surface of killer cells (NK cells, macrophages, etc.), to mediate direct killing of target cells by killer cells. ADCP refers to antibody-dependent cellular phagocytosis, and the mechanism of ADCP is that the target cells acted by antibodies activate the FcγR on the surface of macrophages, induce phagocytosis, make the target cells internalized and degraded by phagosome acidification. Elimination of antibody Fc function is more beneficial in certain specific situations. These situations include the use of antibodies as: (1) receptor agonists to induce cell signaling; (2) Receptor antagonists to block the binding of receptors and ligands and inhibit signaling; or, (3) drug carriers to deliver drugs to target cells expressing the corresponding antigen. If Fc function is maintained, it will lead to the accidental injury of cells expressing corresponding receptors by antibody drugs, as well as the accidental injury of important immune cells by antibody conjugate drugs in the case of off-target.

The combination of Fc variants or mutations is not limited to the following forms (according to EU Numbering):

| IgG | Fc Mutate (EU Numbering) |
|---|---|
| IgG1 | L234A, L235A |
| | L234A, L235A, P329G |
| | L234F, L235E, P331S |
| | D265A, N297A |
| | L234F, L235E, N297A |
| | L234F, L235E, D265A |
| | L234A, L235E, P331S |
| | L234A, L235E, N297A |
| | L234A, L235E, D265A |
| | L234A, L235A, P331S |
| | L234A, L235A, N297A |
| | L234A, L235A, D265A |
| | L235E, D265A, P331S |
| | L235E, N297A, P331S |
| | L235E, N297A |
| | L235A, D265A, P331S |
| | L235A, N297A, P331S |
| | N297Q |
| | N297A |
| | N297G |
| | A287C, N297G, L306C |
| | R292C, N297G, V302C |
| hIgG4 | IS228P, L235E, P329G |
| | S228P, L235E |
| | S228P, F234A, L235E |
| | S228P, F234A, L235A |
| IgG2m4 | ASTKGPSVFPLAPCSRSTSESTAALGCLVKDYFPEPVTVSWNSGALT |
| | SGVHTFPAVLQSSGLYSLSSWTVPSSNFGTQTYTCNVDHKPSNTKV |
| | DKTVERKCCVECPPCPAPPVAGPSVFLFPPKPKDTLMISRTPEVTCW |
| | VDVSQEDPEVQFNWYVDGVEVHNAKTKPREEQFNSTYRVVSVLTV |
| | LHQDWLNGKEYKCKVSNKGLPSSIEKTISKAKGQPREPQWTLPPSQ |
| | EEMTKNQVSLTCLVKGFYPSDIAVEWESNGQPENNYKTTPPVLDSD |
| | GSFFLYSRLTVDKSRWQEGNVFSCSVMHEALHNHYTQKSLSLSLGK |
| | (SEQ ID NO: 90) |
| | N297A |
| | N297Q |
| | N297G. |

At present, murine antibodies are a major source of antibody drugs. Because of their immunogenicity, murine antibodies are generally humanized. The following examples provide murine antibodies, chimeric antibodies, and humanized antibodies. A “chimeric antibody” is an antibody obtained by fusing variable regions of a murine antibody with constant regions of a human antibody, and it can reduce the immune response induced by the murine antibody. The constant regions of the human antibody may be selected from the heavy chain constant region of human IgG1, IgG2, IgG3, IgG4 or variants thereof, and the light chain constant region of human kappa, lambda chain or variants thereof. The “humanized antibody” refers to an antibody obtained by transplanting CDR sequences of a murine antibody into a human antibody variable region framework, and it can overcome the strong reaction induced by a chimeric antibody due to carrying a large number of mouse protein components. Such framework sequences can be obtained from public DNA databases or published references including germline antibody gene sequences. In order to avoid the reduced activity caused by the decrease of immunogenicity, the human antibody variable region framework sequence can be subjected to a minimum of reverse mutation or back mutation to maintain the activity.

Theoretically, the improvement of antibody affinity can contribute to improve the specificity and efficacy of antibodies, reduce the dose of drugs, and reduce toxic side effects, etc. Although actual research work has proved that the relationship between the improvement of affinity and the improvement of antibody titer is not always linear, especially in the treatment of solid tumors, in many cases this linear relationship is obvious. The humanized antibody of the present invention also includes a humanized antibody in which CDRs are further subjected to affinity maturation by phage display. The theoretical basis of antibody affinity maturation in vitro is to mimic the process of antibody affinity in vivo. By constructing a random mutation library to simulate the high frequency mutation of B cells in vivo, high affinity antibodies can be screened.

The drugs provided herein may contain a “therapeutically effective amount” of the antibody or antigen-binding fragment. A “therapeutically effective amount” refers to an amount of a therapeutic agent effective to prevent or ameliorate a particular disease and may vary depending on multiple factors such as the disease state, age, and weight of the patient, and the ability of the agent to produce a desired therapeutic effect in different patients.

“Sequence identity” refers to the sequence similarity between two polynucleotide sequences or between two polypeptides, and the degree to which two polynucleotides or two polypeptides have the same bases or amino acids. As used herein, “having at least 85% sequence identity” refers to achieving at least 85%, 90%, 95%, 97%, or 99% identity.

Antibody-Drug Conjugates (ADCs) refer to binding proteins linked to one or more chemical drugs, which optionally may be therapeutic or cytotoxic agents. An antibody-drug conjugate can be obtained by linking the cytotoxic small molecule (cytotoxin) and the antibody via a permanent or labile chemical linker. ADCs can selectively and sustainably deliver cytotoxic drugs to tumors.

The gene encoding SIRPα is a polymorphic gene, and 10 variants of SIRPα are known in the human population. Katsuto Takenaka et al. sequenced the IgV-encoding SIRP alpha domain of 37 unrelated normal Caucasians, Africans, Chinese, and Japanese from the Human HapMap Genome Project and found 10 different SIRP alpha IgV-encoding alleles (Polymorphism in SIRPα modulates engraftment of human hematopoietic stem cells, NATURE IMMUNOLOGY VOLUME 8 NUMBER 12 Dec. 2007). The 10 SIRPα variants are SIRPα V1/V2/V3/V4/V5/V6/V7/V8/V9/V10 subtypes, respectively. Although SIRPalpha is highly polymorphic, the amino acid sequence alignment of known human SIRPalpha alleles by Chia Chi M. Ho et al showed that there are only two unique sequences at the CD47 binding interface of SIRPalpha, which are allele V1 (a2d1) and V2 (a1d1). (“Velcro” Engineering of High Affinity CD47 Ectodomain as Signal Regulatory Protein (SIRP alpha) Antagonists That Enhance Antibody-dependent Cellular Phagocytosis, JOURNAL OF BIOLOGICAL CHEMISTRY, VOLUME 290•NUMBER 20•May 15, 2015).

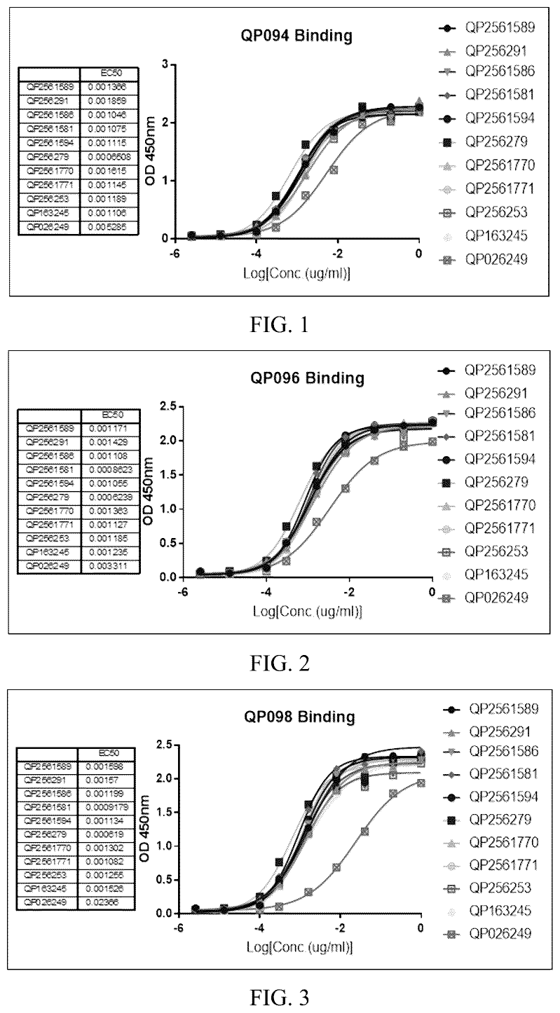

As shown in FIG. 24, the amino acid sequence alignment of known human SIRP alpha binding domain alleles shows only two variations at the CD47-contact interface: a1d1 and a2d1. The first line of text in FIG. 24 is the amino acid sequence of the most significant human SIRP alpha allele V1 (a2d1), and the second line of text in FIG. 1 is the amino acid sequence of the most significant human SIRP allele V2 (a1d1). Black boxes indicate residues that interact with CD47, while shading parts indicate residues that differ from the V1 sequence. Sanger sequencing of SIRPα sequences from 2535 individuals and 510 samples by Janet Sim et al. identified two SIRPα variants v1 and v2, representing three allelic groups: homozygous v1/v1, homozygous v2/v2, and heterozygous v1/v2. The distribution and frequency of SIRPα v1 and v2 allelic groups were determined in different populations and unrelated subpopulations. The distributions of v1/v2 heterozygotes in 5 super populations Europe (EUR), America (AMR), East Asia (EAS), Africa (AFR) and South Asia (SAS) are similar, ranging from 42.0% to 47.2%. The number of v2/v2 in East Asian population is significantly higher than v1/v1, with the occurrence frequencies of 42.3% and 13.3%, respectively. The number of v1/v1 in African, European, American and South Asian population is higher than v2/v2, and the occurrence frequencies of v1 and v2 are 30.3-49.1% and 8.9-24.2%, respectively (see MABS, 2019, VOL. 11, NO. 6, 1036*C1052, https://doi.org/10.1080/19420862.2019.1624123). Aduro Biotech also studied that the occurrence frequency of v2/v2 homozygotes in East Asian population is 41.3% and that of v1/v1 homozygotes is 34.6%, which also proves that 41.3% of East Asian population are V2/V2 homozygotes (see Voets et al. Journal for ImmunoTherapy of Cancer (2019) 7:340).

Based on the results of the SIRPα polymorphism analysis, the ability of anti-SIRPα antibodies to simultaneously bind to both SIRPα v1 and SIRPα v2 genes is critical for clinical development.

The technical solution of the present invention is described in detail below in conjunction with specific embodiments.

The experimental method without specific conditions in the following embodiments is generally in accordance with conventional conditions, or in accordance with the conditions recommended by the raw material or commodity manufacturer, or in accordance with experimental methods recorded in biotechnology textbooks such as Molecular Cloning, Laboratory Manuals, Cold Spring Harbor Laboratory, Contemporary Molecular Biology methods, Cell Biology.

Reagents without specific source specified are conventional reagents purchased through commercial channels.

The molecules and cell lines used in this study are listed in Tables 1 and 2.

TABLE 1
List of Control Antibodies in Pharmacological Studies

| No. | Molecule |
|---|---|
| QP37503751(1H9 | Analog of the anti-SIRPα monoclonal antibody |
| analog) | 1H9 from Forty Seven company |
| QP026249(KAWA23 | Analog of the anti-SIRPα monoclonal antibody |
| analog) | KAWA23 from Forty Seven company |
| QP250251(18D5 | Analog of the anti-SIRPα monoclonal antibody |
| analog) | 18D5 from OSE Immunotherapeutics |
| Tecentriq | Anti-PD-L1 monoclonal antibody Atezolizumab |
| QP11801181 | from Roche Tecentirq analog |

TABLE 2
List of Control Antibodies in Pharmacological Studies

| Cell Name | Description of cell line |
|---|---|
| HCC827 | Human lung cancer cells expressing endogenous PD-L1 |
| U-937 | Human histiocytic lymphoma cells expressing endogenous |
| | SIRPα V1 |
| THP-1 | Human monocytic leukemia cells expressing endogenous |
| | SIRPα V2 |
| Raji | Human Burkitt lymphoma cells expressing endogenous |
| | CD47/CD20 |

### Obtaining Anti SIRPα Antibodies

### Example 1: Obtaining Anti-SIRPα Mouse Antibodies

### (1) Immunization of Mice:

Anti-human SIRPα monoclonal antibodies were generated by immunizing mice. Experimental Balb/c white mice, female, 6 weeks old. Feeding environment: SPF level. After purchase, the mice were kept in laboratory environment for 1 week, with 12/12 hours light/dark cycle adjustment, at the temperature of 20-25° C., and humidity of 40-60%. Balb/c mice were immunized with recombinant protein QP009 (SIRPα) 50 μg/mouse for the first time with complete Freund's adjuvant (CFA). Two weeks later, the mice were alternately immunized with QP009 (SIRPα) in incomplete Freund's adjuvant (IFA) or QP009 (SIRPα) in aluminum salt Alum+CpG ODN 1826 at 25 μg/mouse once a week.

QP009 (SIRPα) has the amino acid sequence shown below (SEQ ID NO:1):

EEELQVIQPDKSVLVAAGETATLRCTATSLIPVGPIQWFRGAGPGRELIYNQKEGHFP RVTTVSDLTKRNNMIDFSIRIGNITPADAGTYYCVKFRKGSPDDVEFKSGAGTELSVRAKP SDYKDDDDKHIIHIHH. The sequence is referenced from UNIPROT number P78324 (31-149) (SIRPA—Tyrosine-protein phosphatase non-receptor type substrate 1 precursor—Homo sapiens (Human)—SIRPA gene & protein (uniprot.org)).

### (2) Cell Fusion:

Mice with high antibody titers in serum were selected for spleen cell fusion. 72 hours prior to fusion, the selected mice were rush immunized by intraperitoneal injection. Spleen lymphocytes were fused with myeloma Sp2/0 cells using an optimized PEG-mediated fusion procedure to obtain hybridoma cells. The fused hybridoma cells were resuspended in HAT complete medium (IMDM medium containing 20% FBS, 1×HAT and 1×OPI), subpackaged in 96-well cell culture plates (1×10^{5/150} μl/well), cultured at 37° C. and 5% CO₂. On the 5^{th} day after fusion, IMDM medium (containing 2×HAT and 1×OPI) containing 20% FBS was added at 50 μl/well, and cultured at 37° C., 5% CO₂. On the 7^{th} to 8^{th} day after fusion, according to the cell growth density, the whole medium was changed with 250 μl/well, and cultured at 37° C., 5% CO₂, wherein the culture medium was HT complete medium (IMDM medium containing 20% FBS, 1×HT and 1×OPI).

### (3) Screening of Hybridoma Cells:

According to the cell growth density, 10-14 days after fusion, ELISA detection was performed to screen the anti-SIRPα antibody in the hybridoma supernatant. The supernatant of hybridoma fusion wells was taken and primarily screened by ELISA in whole 96-well plate, and the anti-SIRPα antibodies in the supernatant that were detected blocking the binding of SIRPα/CD47 were the primary screening positive wells. Then, the supernatant of the primary screening positive wells was taken to detect the binding to QP009 (SIRPα) by ELISA, and the clones that are positive for binding to SIRPα and blocking the binding of SIRPα/CD47 were selected, i.e., the anti-SIRPα antibody positive clone wells. The positive clones were expanded and transferred to the 24/6 wells plate in time, and the cell culture supernatant was detected again by ELISA, and those clone wells positive for binding to SIRPα and blocking the binding of SIRPα/CD47 were the anti-SIRPα antibody positive clone wells. The positive clones were subjected to 2-3 rounds of limited dilution to single-cell clones, and the positive single-cell strain was cryopreserved to obtain single-cell clone 71C10.

### (4) Sequencing of Hybridoma Monoclonal Antibody to Obtain Antibody Sequence:

The positive hybridoma monoclonal cell line 71C10 was taken and mRNA of that was extracted. The mRNA was reverse transcribed into cDNA, and the cDNA was used as a template for PCR amplification. PCR positive clones were selected for sequencing, and the sequences of the light and heavy chain variable regions of monoclonal antibody were obtained by sequence analysis.

The sequence of heavy chain variable region of 71C10 is SEQ ID NO: 2, as follows:

QVQLQQPGTELVRPGASVKLSCKASGYTFTNYWINWVKQRPGQGLEWI
AMIDPSDSETHYNQIFKDKATLTVDKSSNTAYMQLSSLTSGDSAVYYC
AMDYGSLYAMDYWGRGTSVTVSS.

Note: The order is FR1-CDR1-FR2-CDR2-FR3-CDR3-FR4. The bold and underlined parts are VHCDR1 (SEQ ID NO: 3), VHCDR2 (SEQ ID NO: 4), and VHCDR3 (SEQ ID NO: 5), respectively.

The sequence of light chain variable region of 71C10 is SEQ ID NO: 6, as follows:

DIVLTQSPASLAVSLGQRATISCRASKSVSSSGYNYIFWYQQKPGQPP
KLLIYLASNLDSGVPARFSGSGSGTDFTLNIHPVEEEDAATYYCQHSR
ELPTFGGGTKLEIK.

Note: The order is FR1-CDR1-FR2-CDR2-FR3-CDR3-FR4. The bold and underlined parts are VLCDR1 (SEQ ID NO: 7), VLCDR2 (SEQ ID NO: 8), and VLCDR3 (SEQ ID NO: 9), respectively.

### Example 2: Affinity Detection of Anti-SIRPα Chimeric Antibody by SPR

(1) The murine variable region sequences of 71C10 monoclonal cell line were fused with human constant region gene to obtain chimeric antibody molecules. The antibody light chain employs a kappa light chain constant region CL. At the same time, different antigen sequences were designed for the performance test of antibody molecules. The molecular clone designs of antigens and chimeric antibodies are shown in Table 3 and Table 4.

TABLE 3
Molecular clone designs of chimeric antibodies

| Protein | Plasmid | Sequence | | |
|---|---|---|---|---|
| number | number | number | Description | Origin |
| QP026027 | QD026 | SEQ ID NO: 10 | pQDK-KWAR23-L | |
| | QD027 | SEQ ID NO: 11 | pQDH-KWAR23-H | |
| QP026249 | QD026 | SEQ ID NO: 10 | pQDK-KWAR23-L | |
| | QD249 | SEQ ID NO: 12 | pQDH-KWAR23-H(IgG4) | |
| QP163164 | QD163 | SEQ ID NO: 13 | pQDK-180122 VL | 71C10 |
| | QD164 | SEQ ID NO: 14 | pQDH-180122 VH | |
| QP163245 | QD163 | SEQ ID NO: 13 | pQDK-180122 VL | |
| | QD245 | SEQ ID NO: 15 | pQDH-180122 VH (IgG4) | |

Note:
Antibodies with protein numbers QP026027 and QP026249 are used as control antibodies, both of which adopt the variable region sequences of the known anti-SIRPa antibody KWAR23 and are different in the constant regions. The variable regions of monoclonal cell line 71C10 are used in QP163164 and QP163245, and the difference is that the constant regions are different. The sequences shown by the above sequence numbers give the heavy and light chain sequences of each antibody molecule, respectively.

pQD is the name of the vector with the signal peptide and constant region gene (CH1-FC/CL) fragment, wherein pQDH is used for the connection and expression of the heavy chain variable region, and has the signal peptide and constant region gene (CH1-FC) fragment; and pQDK is used for the connection and expression of the light chain variable region, and has the signal peptide and constant region gene (CL) fragment. “H” represents a heavy chain and “L” represents a light chain. “(IgG4)” represents that the heavy chain adopts the constant region of human IgG4. If “(IgG4)” is not indicated, the constant region of human IgG1 is used by default. 180122VH represents the heavy chain variable region derived from the monoclonal cell line 71C10, and 180122VL represents the light chain variable region derived from the monoclonal cell line 71C10.

Exemplarily, “pQDH-KWAR23-H” represents that the control sequence KWAR23 is fused to the pQDH vector, wherein pQDH carries a signal peptide and a constant region gene (CH1-FC) fragment and uses the constant region of human IgG1. “pQDH-180122VH” represents that the heavy chain variable region sequence 180122VH is fused to the pQDH vector, using the constant region of human IgG1. The sequences shown in the above sequence numbers are as follows:

>QD026
(SEQ ID NO: 10)
MDMRVPAQLLGLLLLWFPGSRCQIVLTQSPAIMSASPGEKVTLTCSASSSVSSSYLYW
YQQKPGSSPKLWIYSTSNLASGVPARFSGSGSGTSYSLTISSMEAEDAASYFCHQWSSYPR
TFGAGTKLELKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFNRGEC.

Wherein, the double underlined part is the constant region sequence.

>QD027
(SEQ ID NO: 11)
MEFGLSWLFLVAILKGVQCEVQLQQSGAELVKPGASVKLSCTASGFNIKDYYIHWV
QQRTEQGLEWIGRIDPEDGETKYAPKFQDKATITADTSSNTAYLHLSSLTSEDTAVYYCAR
WGAYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK.

Wherein, the single underlined part is the signal peptide, and the double underlined part is the heavy chain constant region sequence.

>QD249
(SEQ ID NO: 12)
MEFGLSWLFLVAILKGVQCEVQLQQSGAELVKPGASVKLSCTASGFNIKDYYIHWV
QQRTEQGLEWIGRIDPEDGETKYAPKFQDKATITADTSSNTAYLHLSSLTSEDTAVYYCAR
WGAYWGQGTLVTVSSASTKGPSVEPLAPCSRSTSESTAALGCLVKDYFPEPVTVSWNSG
ALTSGVHTFPAVLQSSGLYSLSSVVTVPSSSLGTKTYTCNVDHKPSNTKVDKRVESKYGP
PCPPCPAPEFLGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK

Wherein, the single underlined part is the signal peptide, and the double underlined part is the heavy chain constant region sequence.

>QD163
(SEQ ID NO: 13)
DIVLTQSPASLAVSLGQRATISCRASKSVSSSGYNYIFWYQQKPGQPPKLLIYLASNL
DSGVPARFSGSGSGTDFTLNIHPVEEEDAATYYCQHSRELPTFGGGTKLEIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC.

Wherein, the double underlined part is the constant region sequence.

>QD164
(SEQ ID NO: 14)
QVQLQQPGTELVRPGASVKLSCKASGYTFTNYWINWVKQRPGQGLEWIAMIDPSD
SETHYNQIFKDKATLTVDKSSNTAYMQLSSLTSGDSAVYYCAMDYGSLYAMDYWGRGT
SVTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKENWYVDGVEVHNAKTKPR
EEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPGK.

Wherein, the double underlined part is the constant region sequence.

>QD245
(SEQ ID NO: 15)
MEFGLSWLFLVAILKGVQCQVQLQQPGTELVRPGASVKLSCKASGYTFTNYWINW
VKQRPGQGLEWIAMIDPSDSETHYNQIFKDKATLTVDKSSNTAYMQLSSLTSGDSAVYYC
AMDYGSLYAMDYWGRGTSVTVSSASTKGPSVFPLAPCSRSTSESTAALGCLVKDYFPEP
VTVSWNSGALTSGVHTFPAVLQSSGLYSLSSVVTVPSSSLGTKTYTCNVDHKPSNTKVDK
RVESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK.

Wherein the single underlined part is the signal peptide, and the double underlined part is the heavy chain region sequence.

TABLE 4
Clone designs of antigens

| | Protein | Plasmid | Sequence | |
|---|---|---|---|---|
| | number | number | number | Description |
| Antigen | QP094 | QD094 | SEQ ID | pQD-human SIRPα V1 |
| expression | | | NO: 45 | ECD(E31-R370)-flag-his |
| and | QP096 | QD096 | SEQ ID | pQD-human SIRPα V2 |
| production | | | NO: 46 | ECD(E31-R370)-flag-his |
| | QP098 | QD098 | SEQ ID | pQD-cyno SIRPα(17G9Z7) |
| | | | NO: 47 | ECD(E31-R369)-flag-his |
| | QP100 | QD100 | SEQ ID | pQD-cyno SIRPα(G7PGS8) |
| | | | NO: 48 | ECD(E29-R368)-flag-his |
| | QP271 | QD271 | SEQ ID | pQD-rhesusSIRPα1-flag-his |
| | | | NO: 49 | T-557-28 |
| | QP273 | QD273 | SEQ ID | pQD-cynoSIRPα2-flag-his |
| | | | NO: 50 | T-557-14 |

Note:
QP098 is cynomolgus SIRPα sequence (Uniprot database sequence number I7G9Z7), QP100 is cynomolgus SIRPα sequence (Uniprot database sequence number G7PGS8), QP271 is rhesus monkey SIRPα sequence, which is obtained by the inventor through sequencing monkey PBMC, QP273 is cynomolgus SIRPα sequence, which is obtained by the inventor through sequencing monkey PBMC.

### (2) Expression and Purification of Antigen and Chimeric Antibody

The culture density of 293E cells was maintained at (0.2-3)×10⁶/ml, and a maintenance phase medium (GIBCO Freestyle 293 expression medium) was used for culture; the cells to be transfected were centrifuged one day before transfection, the medium was replaced, and the cell density was adjusted to (0.5-0.8)×10⁶/ml. On the day of transfection, the cell density of 293E cells was (1-1.5)×10⁶/ml. The plasmids and the transfection reagent PEI were prepared. The amount of plasmids to be transfected was 100 ag/100 ml cells, and the mass ratio of PEI to plasmid used was 2:1. The plasmids and PEI were mixed uniformly, then standing for 15 min (should not exceed 20 min). The mixture of plasmids and PEI was slowly added to 293E cells, and cultured in a shaker at 8% CO₂, 120 rpm and 37° C. On the fifth day of transfection, the cell supernatant was collected after being centrifuged in a horizontal centrifuge at 4700 rpm for 20 min.

Protein A affinity chromatography purification: At least 3CV (actual volume 20 ml) of balance solution was allowed to pass through the column, so as to ensure that the pH and conductivity of the final solution flowing out of the instrument are consistent with the balance solution, and the flow rate was 1 ml/min; the culture supernatant after centrifugation was allowed to pass through the column, 40 ml of sample was loaded, and the flow rate was 0.33 ml/min; at least 3CV (actual volume 20 ml) of balance solution was allowed to pass through the column, so as to ensure that the pH and conductivity of the final solution flowing out of the instrument are consistent with the balance solution, and the flow rate was 0.33 ml/min; the eluent was allowed to pass through the column, and the elution peaks (PAC-EP) began to be collected when the UV280 increased to 15 mAU; and collection stopped when the UV280 decreased to 15 mAU, and the flow rate was 1 ml/min. After the sample collection was completed, the PAC-EP was adjusted to neutral with a pH adjustment solution.

### (3) Affinity Detection by Surface Plasmon Resonance (SPR)

The affinity of the anti-SIRPα chimeric antibody QP163164 to human SIRPα V1 (protein number QP094) and human SIRPα V2 (protein number QP096) was determined by Biacore T200 (GE). Tables 5 and 6 show the detection results of QP163164 and QP026027. The results show that the SIRPα chimeric antibody QP163164 binds to human SIRPα V1 with a KD of 5.27E-10M, and binds to human SIRPα V2 with a KD of 6.78E-10M. The binding affinity to human SIRPα V1 and human SIRPα V2 is significantly better than the control antibody KWAR23 (QP026027).

TABLE 5
Affinity of anti-SIRPα chimeric antibodies to SIRPα V1 and SIRPα V2

| | SIRPα V1(QP094) | | | SIRPα V2(QP096) | | |
|---|---|---|---|---|---|---|
| No. | ka(1/Ms) | Kd(1/s) | KD (M) | ka(1/Ms) | Kd(1/s) | KD (M) |
| QP163164 | 2.71E+06 | 1.43E−03 | 5.27E−10 | 3.39E+06 | 2.30E−03 | 6.78E−10 |
| QP026027 | 9.08E+05 | 4.65E−03 | 5.12E−09 | 2.01E+06 | 2.46E−02 | 1.23E−08 |

The affinity of the chimeric antibodies to cynomolgus SIRPα determined by biacore is as shown in the following table:

TABLE 6
Affinity of chimeric antibodies to cynomolgus SIRPα

| | Cynomolgus SIRPα (QP100) | | |
|---|---|---|---|
| No. | ka(1/Ms) | Kd(1/s) | KD (M) |
| QP163164 | 1.99E+06 | 2.63E−01 | 1.32E−07 |
| QP026027 | 3.13E+06 | 1.99E−02 | 6.35E−09 |

### Example 3: Humanization of Anti-SIRPα Hybridoma Monoclonal Antibodies

By means of aligning the IMGT germline gene database of the heavy and light chain variable regions of human antibody and using MOE software, the heavy and light chain variable region germline genes having high homology with QP163164 were selected as templates, and the CDRs of murine antibodies were grafted into the corresponding human templates to form a variable region sequence in the order of FR1-CDR1-FR2-CDR2-FR3-CDR3-FR4. Then, some important amino acid residues were selected for reverse mutation combinations. Among them, the amino acid residues were identified and annotated by Kabat numbering system. In the following examples, the heavy chain FR region sequences are derived from the combined sequences of human germline heavy chains IGHV1-18 and IGHJ2*01, which comprise the FR1, FR2, FR3 regions of human germline heavy chain IGHV1-18 and the FR4 region of human germline heavy chain IGHJ2*01. The light chain FR region sequences are derived from the combined sequences of human germline light chains IGKV4-1 and IGKJ2*01, which comprise the FR1, FR2, FR3 regions of human germline light chain IGKV4-1 and the FR4 region of human germline light chain IGKJ2*01.

### (1) Cloning of Anti-SIRPα Antibody Humanized Molecules

Primers were designed for PCR to construct VH/VK gene fragments of various humanized antibodies, and then homologous recombination was carried out with the expression vector pQD having a signal peptide and a constant region gene (CH1-FC/CL) fragments to construct the antibody full-length expression vector VH-CH1-FC-pQD/VK-CL-pQD.

The online software DNAWorks (v3.2.2) (http://helixweb.nih.gov/dnaworks/) was used to design a number of primers to synthesize VH/VK containing the gene fragment needed for recombination: 5′-30 bp signal peptide+VH/VK+30 bp CH1/CL-3′. According to the operating instructions of Primer STAR GXL DNA polymerase of TaKaRa, VH/VK containing the gene fragment needed for recombination was obtained by two-step PCR using the multiple primers designed above. Construction and enzyme digestion of expression vector pQD: the characteristic of a number of restriction enzymes, such as BsmBI, that their recognition sequences differ from the digestion sites was used to design and construct the expression vector pQD. The vector was digested using BsmBI enzyme, and the gel was cut and recovered for later use. Construction of heavy chain expression vector pQD-VH-CH1-FC and light chain expression vector pQD-VL-CL: heavy chain variable region VH gene fragments were mixed with BsmBI-digested vector pQD (with a signal peptide and a heavy chain constant region (CH1-FC) fragment) at a ratio of 3:1; light chain variable region VL gene fragments were mixed with BsmBI-digested vector pQD (with a signal peptide and a light chain constant region (CL) fragment) at a ratio of 3:1; the mixtures were transferred into DH5a competent cells respectively, then subjected to ice bath at 0° C. for 30 min, heat shock at 42° C. for 90 s, then added with 5 times of LB medium, incubated at 37° C. for 45 min, coated to LB-Amp plate, and cultured overnight at 37° C., and single clones were picked for sequencing to obtain individual clones of interest.

The specific information of the humanization design for QP163164 is shown in the Table below. The protein expression number is QP256253. In this table, a kappa light chain constant region CL is employed for the antibody light chain, and a human IgG4 constant region is employed for the antibody heavy chain (see Example 2 for the specific sequences of constant regions). The humanized design light and heavy chain variable region sequences are not limited to the sequences shown in the following table.

TABLE 7
Humanized design light and heavy chain sequences and protein expression number

| | | Heavy | | | Light | | |
|---|---|---|---|---|---|---|---|
| | | chain | | | chain | | |
| | Protein | plasmid | Sequence | | plasmid | Sequence | |
| | number | number | number | Description | number | number | Description |
| QP163164 | QP256253 | QD256 | SEQ ID | pQDH (IgG4)- | QD253 | SEQ ID | pHrK- |
| humanized | | | NO: 17 | 164_VH.1B | | NO: 16 | 164_VL.1A |
| clone | | | | | | | |

Note:
The light chain variable region of QP256253 is encoded by the plasmid numbered QD253.

The specific sequence of light chain variable region SEQ ID NO:16 is:

DIVLTQSPDSLAVSLGERATINCRASKSVSSSGYNYIFWYQQKPGQPP
KLLIYLASNLDSGVPDRFSGSGSGTDFTLTISSLQAEDVAVYYCQHSR
ELPTFGQGTKLEIK.

The heavy chain variable region of QP256253 is encoded by the plasmid numbered QD256. The specific sequence of heavy chain variable region SEQ ID NO:17 is:

QVQLVQSGAEVKKPGASVKVSCKASGYTFTNYWINWVRQAPGQGLEWI
AMIDPSDSETHYNQIFKDRATLTTDTSTSTAYMELRSLRSDDTAVYYC
AMDYGSLYAMDYWGRGTLVTVSS.

### (2) Expression of Humanized Anti-SIRPα Antibody Proteins

The culture density of 293E cells was maintained between (0.2-3)×10⁶/ml, and maintenance phase medium (GIBCO Freestyle 293 expression medium) was used for culture; the cells to be transfected were centrifuged one day before transfection, the medium was replaced, and the cell density was adjusted to (0.5-0.8)×10⁶/ml. On the day of transfection, the cell density of 293E cells was (1-1.5)×10⁶/ml. The plasmid and transfection reagent PEI were prepared, the amount of plasmid to be transfected was 100 μg/100 ml cells, and the mass ratio of PEI to the plasmid as used was 2:1. The plasmid and PEI were mixed uniformly, then allowed to stand for 15 min (should not exceed 20 min). The mixture of plasmids and PEI was slowly added to 293E cells, and cultured in a shaker at 8% CO₂, 120 rpm and 37° C. On the fifth day of transfection, the cell supernatant was collected by centrifugation at 4700 rpm for 20 min in a horizontal centrifuge.

### (3) Purification of Anti-SIRPα Antibody Humanized Protein

Protein A affinity chromatography purification: At least 3CV (actual volume 20 ml) of balance solution was allowed to pass through the column, so as to ensure that the pH and conductivity of the final solution flowing out of the instrument are consistent with the balance solution, and the flow rate was 1 ml/min; the culture supernatant after centrifugation was allowed to pass through the column, 40 ml of sample was loaded, and the flow rate was 0.33 ml/min; at least 3CV (actual volume 20 ml) of balance solution was allowed to pass through the column, so as to ensure that the pH and conductivity of the final solution flowing out of the instrument are consistent with the balance solution, and the flow rate was 0.33 ml/min; the eluent was allowed to pass through the column, and the elution peaks (PAC-EP) began to be collected when the UV280 increased to 15 mAU; and collection stopped when the UV280 decreased to 15 mAU, and the flow rate was 1 ml/min. After the sample collection was completed, the PAC-EP was adjusted to neutral with a pH adjustment solution.

### (4) Identification of Humanized SIRPα Antibody Activity (Binding-ELISA)

Binding-ELISA experimental method: The plate was coated with QP094 (SIRPαV1-flag-his), QP096 (SIRPαV2-Flag-his), and QP100 (cynoSIRPα-flag-his) at 0.5 μg/ml, 50 μl/well, respectively, for 4° C. overnight. The plate was washed with PBS for 3 times, added with 3% BSA/PBS at 200 μl/well, and incubated at RT for 2 h, then washed with PBST for 3 times. Antibodies with different concentrations were added, and incubated for 1 h at RT, then the plate was washed with PBST for 3 times and with PBS for 3 times. Secondary antibody RP-anti Fab diluted at 1:2500 was added and incubated at RT for 1 h, then the plate was washed with PBST for 3 times and with PBS for 3 times. TMB was used to develop and 2M H₂SO₄ was used to terminate, and reading was performed at 450 nm.

### (5) Identification of Humanized SIRPα Antibody Affinity by SPR

The affinity of the humanized antibody to human SIRPα V1, human SIRPα V2 and cynomolgus SIRPα was determined by Biacore. The results are as shown in Table 6 below. The results show that the anti-SIRPα humanized antibody QP256253 binds to human SIRPα V1 with a KD value of 3.36E-10M, and binds to human SIRPα V2 with a KD value of 3.19E-10M.

TABLE 8
Affinity of the humanized antibody to human SIRPα V1 and SIRPα V2 and cynomolgus SIRPα

| | QP094 (human SIRPα V1) | | | QP096 (human SIRPα V2) | | | QP100 (cyno SIRPα) | | |
|---|---|---|---|---|---|---|---|---|---|
| Number | ka(1/Ms) | Kd(1/s) | KD (M) | ka(1/Ms) | Kd(1/s) | KD (M) | ka(1/Ms) | Kd(1/s) | KD (M) |
| QP256253 | 2.35E+06 | 7.90E−04 | 3.36E−10 | 3.42E+06 | 1.09E−03 | 3.19E−10 | 2.52E+06 | 1.96E−01 | 7.79E−08 |

### Example 4: Affinity Maturation of Anti-SIRPα Antibody QP163164

### (1) Construction of Humanized Phagemid Vector

Humanized QP256253 was constructed into a phagemid vector in scFv mode (VH-3×GGGGS-VL) as the wild-type sequence (i.e., as the original or initial sequence, and the sequences obtained by affinity maturation screening were mutant sequences). VH, (GGGGS)3 linker, and VL were spliced by over-lap PCR, and were ligated into phagemid vectors via NcoI and NotI restriction sites.

### (2) Construction of Phage Display Library

The constructed wild-type scFv was used as a template, and codon-based primers were used. In the process of primer synthesis, there was 50% wild-type codon and 50% NNK (reverse primer: MNN) in the codon of the mutation region, and the mutation library was constructed by introducing mutations into all CDR regions. The PCR fragment was digested by NcoI and NotI enzymes, ligated into the phagemid vector, and finally electrotransformed into E. coli TG1. Each codon-based primer was used to establish an independent library.

### (3) Library Screening

After the library was rescued to package the phage particles for screening, biotinylated QP098 (cynoSIRPα (ECD)) antigen and streptavidin magnetic beads were used for liquid phase screening, and the antigen concentration was reduced in each round of screening relative to that in the previous round. After three rounds of screening, 250 clones were picked for phage ELISA detection of binding activity, and the positive clones were sequenced. After alignment analysis of the sequenced clones and removal of redundant sequences, the non-redundant sequences were converted into full-length IG (CH1-CH2-CH3 of hIgG4 was selected for heavy chain constant region; kappa (κ) light chain CL was selected for light chain constant region) for expression in mammalian cells. Full length IG protein was obtained after affinity purification. The specific sequence is shown in the table below. In this table, a kappa light chain constant region CL is employed for the antibody light chain, and a human IgG4 constant region is employed for the antibody heavy chain (see Example 2 for the specific sequences of constant regions).

TABLE 9

| QP163164 | | Heavy chain | | Light chain | |
|---|---|---|---|---|---|
| affinity | Protein | plasmid | Sequence | plasmid | Sequence |
| maturation | number | number | number | number | number |
| | QP256279 | QD256 | SEQ ID | QD279 | SEQ ID NO: 18 |
| | | | NO: 17 | | |
| | QP256291 | QD256 | SEQ ID | AD291 | SEQ ID NO: 19 |
| | | | NO: 17 | | |
| | AP2561581 | QD256 | SEQ ID | QD1581 | SEQ ID NO: 20 |
| | | | NO: 17 | | |
| | QP2561586 | QD256 | SEQ ID | QD1586 | SEQ ID NO: 21 |
| | | | NO: 17 | | |
| | QP2561589 | QD256 | SEQ ID | QD1589 | SEQ ID NO: 22 |
| | | | NO: 17 | | |
| | QP2561594 | QD256 | SEQ ID | QD1594 | SEQ ID NO: 23 |
| | | | NO: 17 | | |
| | QP2561770 | QD256 | SEQ ID | QD1770 | SEQ ID NO: 24 |
| | | | NO: 17 | | |
| | QP2561771 | QD256 | SEQ ID | QD1771 | SEQ ID NO: 25 |
| | | | NO: 17 | | |

Note:
The naming rule of protein number is the combination of heavy chain plasmid number
and light chain plasmid number. Exemplarily, the anidbody molecule whose protein
number is QP256279 has a heavy chain plasmid number of QD256 and a light chain
plasmid number of QD279. The sequences represented by the sequence numbers in the
table are heavy chain variable region or light chain variable region sequences of
different antibodies. The specific sequences of light chain varable regions are as
follows.
>QD279 (SEQ ID NO: 18)
DIVLTQSPDSLAVSLGERATINCRASQSVRSSGYNWIFWYQQKPGQPPKLLIYLASN
RDSGVPDRFSGSGSGTDFTLTISSLQAEDVAVYYCQHSRELPTFGQGTKLEIK.
>QD291 (SEQ ID NO: 19)
DIVLTQSPDSLAVSLGERATINCRASKSVGSSGYNWLFWYQQKPGQPPKLLIYLAS
NRDSGVPDRFSGSGSGTDFTLTISSLQAEDVAVYYCQHSRELPTFGQGTKLEIK.
>QD1581 (SEQ ID NO: 20)
DIVLTQSPDSLAVSLGERATINCRASKSVSSSGYNYIFWYQQKPGQPPKLLIYLASNL
DPGVPDRFSGSGSGTDFTLTISSLQAEDVAVYYCQHSRELPTFGQGTKLEIK.
>QD1586 (SEQ ID NO: 21)
DIVLTQSPDSLAVSLGERATINCRASKSVSSSGYNYIFWYQQKPGQPPKLLIYLASNL
DSGVPDRFSGSGSGTDFTLTISSLQAEDVAVYYCQESRELPTFGQGTKLEIK.
>QD1589 (SEQ ID NO: 22)
DIVLTQSPDSLAVSLGERATINCRASKSVSSSGYNYIFWYQQKPGQPPKLLIYLASNL
DSGVPDRFSGSGSGTDFTLTISSLQAEDVAVYYCQESWELPTFGQGTKLEIK.
>QD1594 (SEQ ID NO: 23)
DIVLTQSPDSLAVSLGERATINCRASKSVSSSGYNYIFWYQQKPGQPPKLLIYLASNL
DSGVPDRFSGSGSGTDFTLTISSLQAEDVAVYYCQHSRDLPTFGQGTKLEIK.
>QD1770 (SEQ ID NO: 24)
DIVLTQSPDSLAVSLGERATINCRASqSVrSSGYNwIFWYQQKPGQPPKLLIYLASNrD
SGVPDRFSGSGSGTDFTLTISSLQAEDVAVYYCQeSRELPTFGQGTKLEIK.
>QD1771 (SEQ ID NO: 25)
DIVLTQSPDSLAVSLGERATINCRASKSVSSsGYNwIFWYQQKPGQPPKLLIYLASNr
DSGVPDRFSGSGSGTDFTLTISSLQAEDVAVYYCQeSRELPTFGQGTKLEIK.

The above bold and underlined parts are VLCDR1, VLCDR2, VLCDR3 of each antibody molecule, respectively, and the comparison with the wild-type sequence QP256253 is as follows:

TABLE 10
Comparison of LCDR region sequences of each antibody molecule with wild-type
sequences

| | Light chain | | | |
|---|---|---|---|---|
| Protein | plasmid | | | |
| number | number | VLCDR1 | VLCDR2 | VLCDR3 |
| QP256253 | QD253 | RASKSVSSSGYNYIF | LASNLDS | QHSRELPT |
| | | (SEQ ID NO: 7) | (SEQ ID NO: 8) | (SEQ ID NO: |
| | | | | 9) |
| QP256279 | QD279 | RASQSVRSSGYNWIF | LASNRDS | / |
| | | (SEQ ID NO: 37) | (SEQ ID NO: | |
| | | | 38 | |
| QP256291 | QD291 | RASKSVGSSGYNWLF | LASNRDS | / |
| | | (SEQ ID NO: 39) | (SEQ ID NO: | |
| | | | 38) | |
| QP2561581 | QD1581 | / | LASNLDP | / |
| | | | (SEQ ID NO: | |
| | | | 40) | |
| QP2561586 | QD1586 | / | / | QESRELPT |
| | | | | (SEQ ID NO: |
| | | | | 41) |
| QP2561589 | QD1589 | / | / | QESWELPT |
| | | | | (SEQ ID NO: |
| | | | | 42) |
| 0QP2561594 | QD1594 | / | / | QHSRDLPT |
| | | | | (SEQ ID NO: |
| | | | | 43) |
| QP2561770 | QD1770 | RASQSVRSSGYNWIF | LASNRDS | QESRELPT |
| | | (SEQ ID NO: 37) | (SEQ ID NO: | (SEQ ID NO: |
| | | | 38) | 41) |
| QP2561771 | QD1771 | RASKSVSSSGYNWIF | LASNRDS | QESRELPT |
| | | (SEQ ID NO: 44) | (SEQ ID NO: | (SEQ ID NO: |
| | | | 38) | 41) |

Note:
″/″ indicates that the sequence is the same as QP256253, and bold indicates different amino acids from QD253.

### (4) ELISA Assay

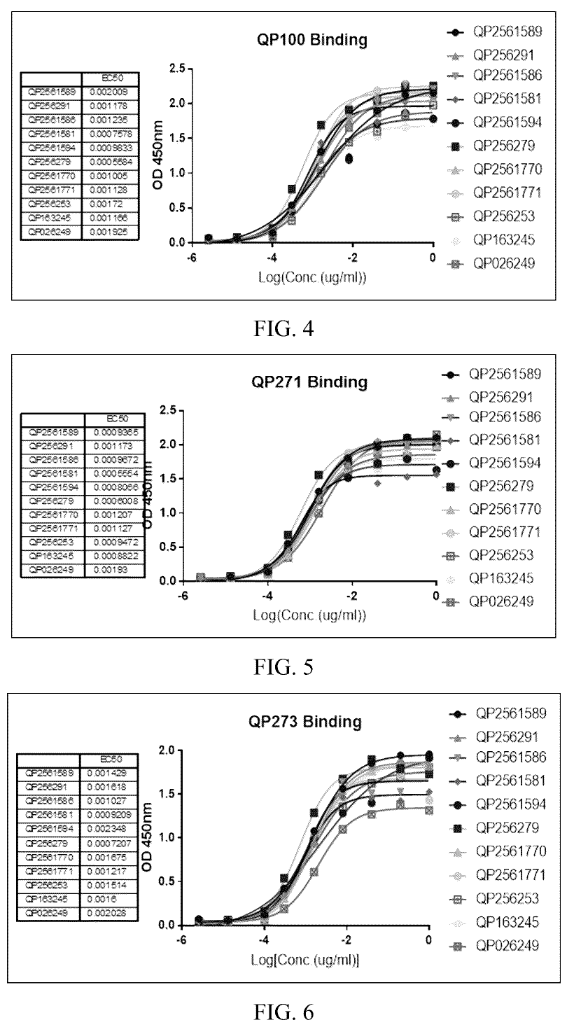

Binding-ELISA experimental method: The plate was coated with QP094 (SIRPαV1-flag-his), QP096 (SIRPαV2-Flag-his), QP098 (cynoSRP-flag-his), and QP100 (cynoSRP 8-flag-his) at 0.5 μg/ml, 50 μl/well, respectively, for 4° C. overnight. The plate was washed with PBS for 3 times, added with 300 BSA/PBS at 200 μl/well, and incubated at RT for 2 h, then washed with PBST for 3 times. Antibodies with different concentrations were added, and incubated for 1 h at RT, then the plate was washed with PBST for 3 times and with PBS for 3 times. Secondary antibody FWR-anti Fab diluted at 1:2500 was added and incubated at RT for 1 h, then the plate was washed with PB ST for 3 times and with PBS for 3 times. TMB was used to develop and 2M H₂SO₄ was used to terminate, and reading was performed at 450 nm. The EC50 values are shown in the table below. The following table also shows the detection results of humanized antibody QP256253, chimeric antibody QP163245, and control antibody QP026249. The results are shown in FIGS. 1 to 6.

TABLE 10
EC50 values detected by ELISA

| conc.(μg/ml) | QP094 | QP096 | QP098 | QP100 | QP271 | QP273 |
|---|---|---|---|---|---|---|
| QP2561589 | 0.001366 | 0.001171 | 0.001598 | 0.002009 | 0.0009365 | 0.001429 |
| QP256291 | 0.001859 | 0.001429 | 0.00157 | 0.001178 | 0.001173 | 0.001618 |
| QP2561586 | 0.001046 | 0.001108 | 0.001199 | 0.001235 | 0.0009672 | 0.001027 |
| QP2561581 | 0.001075 | 0.0008623 | 0.0009179 | 0.0007578 | 0.0005554 | 0.0009209 |
| QP2561594 | 0.001115 | 0.001055 | 0.001134 | 0.0009833 | 0.0008066 | 0.002348 |
| QP256279 | 0.0006508 | 0.0006239 | 0.000619 | 0.0005584 | 0.0006008 | 0.0007207 |
| QP2561770 | 0.001615 | 0.001363 | 0.001302 | 0.001005 | 0.001207 | 0.001675 |
| QP2561771 | 0.001145 | 0.001127 | 0.001082 | 0.001128 | 0.001127 | 0.001217 |
| QP256253 | 0.001189 | 0.001185 | 0.001255 | 0.00172 | 0.0009472 | 0.001514 |
| QP163245 | 0.001106 | 0.001235 | 0.001526 | 0.001166 | 0.0008822 | 0.0016 |
| QP026249 | 0.005285 | 0.003311 | 0.02366 | 0.001925 | 0.00193 | 0.002028 |

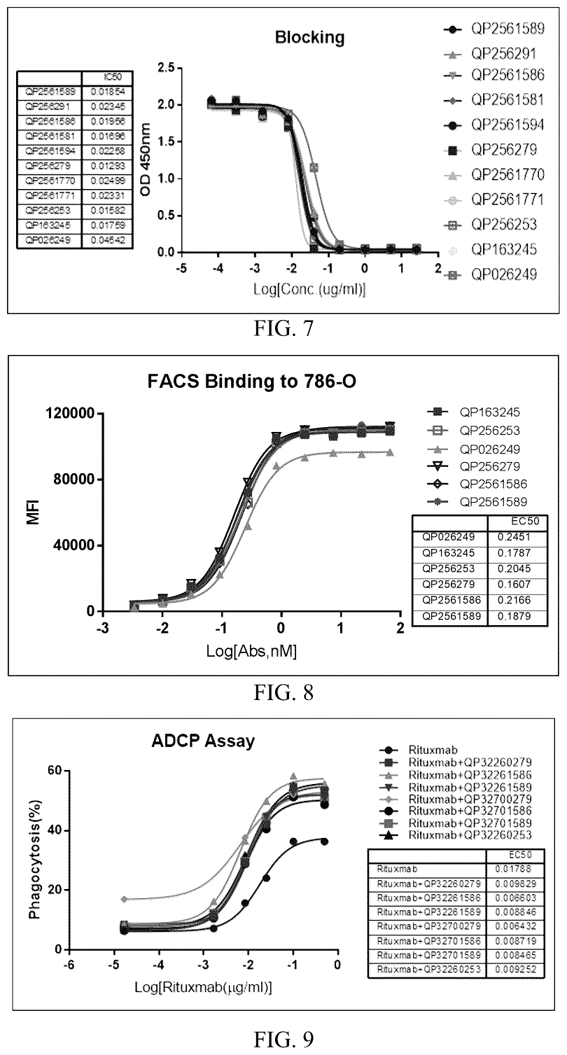

Blocking-ELISA experimental method: the plate was coated with QP001.2 at 2 μg/ml for 4° C. overnight, washed with PBS for 3 times, and blocked with 5% milk at 250 μl/well. Biotin-QP002 0.05 μg/ml+Abs 50 μg/ml were mixed at 1:1 and incubated at 25° C. for 1 h with HRP-Strepavidin (1:5000). The results are shown in FIG. 7

### (5) Affinity Detection by Surface Plasmon Resonance (SPR)

The affinity of the anti-SIRPα antibodies to human SIRPα V1 type, human SIRPα V2 type and cynomolgus SIRPα was determined by Biacore, and some of the results are shown in Table 10. As shown in Table 10, the anti-SIRP1 antibodies QP2561589, QP2561586, QP2561581, QP256279, and QP2561770 all bind to human SRP1 V1 type and human SIRPα V2 type. At the same time, QP2561589, QP2561586, QP256279, QP2561770, QP256253 all bind to different cynomolgus and rhesus monkey SIRPα proteins.

TABLE 11
Results of Affinity detected by SPR

| | Biacore (KD/M) | | | | |
|---|---|---|---|---|---|
| | QP094 | QP096 | QP098 | QP271 | QP273 |
| | (Human | (Human | (cyno- | (rhesus | (cyno- |
| | SIRPα | SIRPα | molgus | monkey | molgus |
| Protein | V1) | V2) | SIRPα) | SIRPα) | SIRPα) |
| QP2561589 | 8.50E−11 | 3.92E−11 | 6.03E−09 | 2.41E−09 | 3.20E−09 |
| QP2561586 | 5.97E−11 | 8.91E−11 | 4.22E−09 | 1.74E−09 | 2.22E−09 |
| QP2561581 | 1.37E−10 | 1.76E−10 | / | / | / |
| QP256279 | 3.54E−11 | 4.46E−11 | 1.56E−09 | 4.41E−10 | 9.18E−10 |
| QP2561770 | 2.88E−11 | 3.68E−11 | 2.56E−10 | 1.00E−10 | 1.63E−10 |
| QP256253 | 2.60E−10 | 2.76E−10 | 2.68E−08 | 7.97E−09 | 1.27E−08 |
| QP163245 | 3.86E−10 | 5.58E−10 | / | / | / |
| QP026249 | 3.43E−09 | 1.36E−08 | 2.71E−08 | / | / |

As can be seen from the above table, the affinity of the affinity mature antibodies QP2561589, QP2561586 and QP256279 proteins for human SIRPα V1 and SIRPα V2 is more than 50 times higher than that of the control antibody KWAR23 (QP026249).

### Example 5: FACS Detection of Anti-SIRPα Antibody Binding to Human Renal Clear Cell Adenocarcinoma Cells 786-0 Cells Naturally Expressing Human SIRPα

Experimental steps: 786-0 cells were collected at 2E5 cells/well, washed with PBS once, then centrifuged at 300 g for 3 min and the supernatant was discarded. Blocking: the cells were resuspended with 2% FBS and were seeded into 96-well U bottom plate with 2E5/well, 200 μl/well, then subjected to ice bath for 1 h. Centrifugation was performed at 300 g for 3 min, and the supernatant was discarded. Antibody incubation: 10 μg/ml antibodies for incubation were diluted at 1:3, 100 μl/well, and subjected to ice bath for 1 h. Centrifugation was performed, and the supernatant was discarded. Pre-cooled PBS was added at 200 μl/well, centrifuged at 300 g for 5 min to discard supernatant, which was repeated twice. Secondary antibody: PE-anti human FC (1:200) 50 μl/well was subjected to ice bath for 0.5 h. Centrifugation was performed and the supernatant was discarded. Pre-cooled PBS was added at 200 μl/well, centrifuged at 300 g for 5 min to discard supernatant, which was repeated 3 times. The mean fluorescence values were read on FACS. The results are shown in FIG. 8, the anti-SIRPα antibodies QP163245, QP256253, QP256279, QP2561586, QP2561589 all bind to human renal clear cell adenocarcinoma cells 786-0 cells naturally expressing human SIRPα, and the binding affinity is much better than that of the control antibody QP026249 (KWAR23).

### Example 6: In Vitro Functional Assay ADCP of Anti-SIRPα Antibody

(1) The Anti-SIRPα Antibody was Made into Different IgG Subtypes, and the Molecular Cloning Designs were as Follows

TABLE 12
Molecular cloning designs for in vitro functional assay ADCP of anti-SIRPα antibody

| | | Protein | Heavy chain | Sequence | Light chain | Sequence |
|---|---|---|---|---|---|---|
| | IgG subtype | number | plasmid number | number | plasmid number | number |
| Different | IgG1 (N297Q) | QP32250279 | QD3225 | SEQ ID NO: 27 | QD279 | SEQ ID NO: 18 |
| subtypes of | IgG4 (N297Q) | QP32260279 | QD3226 | SEQ ID NO: 28 | QD279 | SEQ ID NO: 18 |
| QP256279 | IgG2 | QP32550279 | QD3255 | SEQ ID NO: 29 | QD279 | SEQ ID NO: 18 |
| | IgG1 (L234A, L235A) | QP32600279 | QD3260 | SEQ ID NO: 30 | QD279 | SEQ ID NO: 18 |
| | IgG1 (L234A, L235A, | QP32650279 | QD3265 | SEQ ID NO: 31 | QD279 | SEQ ID NO: 18 |
| | P329G) | | | | | |
| | IgG1 (L234A, L235A, | QP32700279 | QD3270 | SEQ ID NO: 26 | QD279 | SEQ ID NO: 18 |
| | K338A) | | | | | |
| Different | IgG4 (N297Q) | QP32261586 | QD3226 | SEQ ID NO: 28 | QD1586 | SEQ ID NO: 21 |
| subtypes of | IgG1 (L234A, L235A, | QP32701586 | QD3270 | SEQ ID NO: 26 | QD1586 | SEQ ID NO: 21 |
| QP2561586 | K338A) | | | | | |
| Different | IgG4 (N297Q) | QP32261589 | QD3226 | SEQ ID NO: 28 | QD1589 | SEQ ID NO: 22 |
| subtypes of | IgG1 (L234A, L235A, | QP32701589 | QD3270 | SEQ ID NO: 26 | QD1589 | SEQ ID NO: 22 |
| QP2561589 | K338A) | | | | | |
| Different | IgG1 (L234A, L235A, | QP32680026 | QD3268 | SEQ ID NO: 32 | QD026 | SEQ ID NO: 10 |
| subtypes of | K338A) | | | | | |
| KWAR23 | IgG4 (N297Q) | QP32210026 | QD3221 | SEQ ID NO: 35 | QD026 | SEQ ID NO: 10 |
| Different | IgG1 (L234A, L235A, | QP32713240 | QD3271 | SEQ ID NO: 33 | QD3240 | SEQ ID NO: 36 |
| subtypes of | K338A) | | | | | |
| Aduro 50A | | | | | | |
| Different | IgG1 (N297Q) | QP32220163 | QD3222 | SEQ ID NO: 34 | QD163 | SEQ ID NO: 13 |
| subtypes of | | | | | | |
| QP163164 | | | | | | |
| Different | IgG1 (N297Q) | QP32250253 | QD3225 | SEQ ID NO: 27 | QD253 | SEQ ID NO: 16 |
| subtypes of | IgG4 (N297Q) | QP32260253 | QD3226 | SEQ ID NO: 28 | QD253 | SEQ ID NO: 16 |
| QP256253 | | | | | | |

Note: The naming rule of protein number is the combination of heavy chain plasmid number and light chain plasmid number. The sequence numbers of the heavy chains show the heavy chain sequences of different subtype antibodies. The sequence numbers of light chains show the sequences of light chains or light chain variable regions of different subtype antibodies. (L234A, L235A, K338A) refers to the FC segment mutation that eliminates FC γ R function (EU count L234A/L235A/K338A).

Among them, the specific sequence of the heavy chain of QP32700279 (SEQ ID NO: 26) is as follows

QVQLVQSGAEVKKPGASVKVSCKASGYTFTNYWINWVRQAPGQGLEWIAMIDPSD
SETHYNQIFKDRATLTTDTSTSTAYMELRSLRSDDTAVYYCAMDYGSLYAMDYWGRGTL
VTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISAAKGQPREPQVYTLP
PSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK.

The sequence of the light chain variable region of QP32700279 is shown in SEQ ID NO: 18.

### (2) In Vitro Functional Assay ADCP of Anti-SIRPα Antibody

Preparation of macrophages: PBMCs were resuscitated, monocytes were isolated with EasySep™ Human Monocyte Isolation Kit (Stemcell-19359), Human Recombinant M-CSF (final concentration of 50 ng/mL) was added and mixed evenly. Cells were cultured at 37° C. for 6 days to be induced into macrophages, and the cells were collected and counted for later use. Raji cells were labeled with CFSE. Raji cells were resuspended to 2×10⁶ cells/ml and added to the 96-well plate with macrophages at 50 μl/well (1×10⁵/well). Dilution of antibody: Rituximab was diluted to 80 μg/ml with complete medium, and diluted with 3 times to 9 gradients; anti-SIRPα antibodies were diluted to 20 μg/ml with complete medium; Mixture of Antibody: the diluted two kinds of antibodies were mixed at a ratio of 1:1 in the Combination group, and the antibodies were mixed with equal volume of culture medium in the Rituximab group, then added them into the 96-well plate previously seeded with cells at 50 μl/well and cultured at 37° C. for 2 h; FACS detection: phagocytosis was measured by gating live CFSE+/CD14+ cells.

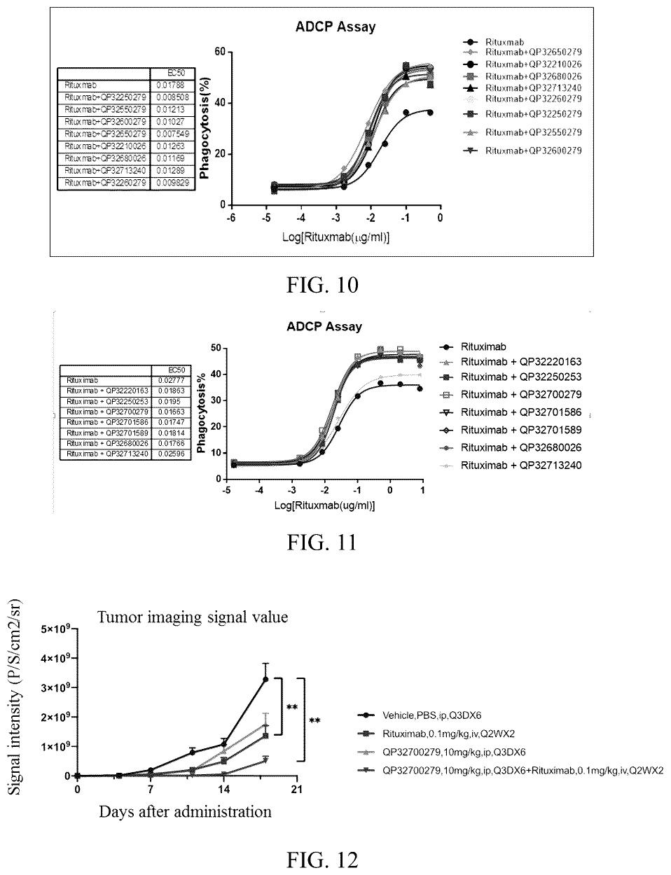

The affinity mature molecule and the control antibody were used for ADCP assay in cooperation with Rituximab, and the experimental results show that the combination of anti-SIRPα antibody and Rituximab has a smaller EC50 and a significantly greater synergistic effect of ADCP than Rituximab alone. The results are shown in FIGS. 9, 10 and 11.

### Example 7: Evaluation of the Inhibition of Raji-Luc Tumor Growth by Anti-SIRPα Antibody QP32700279 in the B-NDG-hSIRPA Mouse Model

In order to investigate the tumor killing effect of anti-SIRPα antibody, the Raji-Luc tumor model was inoculated intravenously with B-NDG-hSIRPα to evaluate the inhibitory effect of SIRPα antibodies and Rituximab on tumor growth. Raji-Luc cells were cultured in RPMI1640 medium containing 10% fetal bovine serum. PBS-resuspended Raji-Luc cells were inoculated into the tail vein of the B-NDG-hSIPRa mouse at a concentration of 5×10⁵ cells/0.2 mL in a volume of 0.2 mL/mouse. On day 0 and day 3 after inoculation, the tumor imaging signal value was measured with a small animal imager. When the average imaging signal intensity reached about 1×10⁶ P/S, appropriate animals were selected according to the tumor imaging signal value and animal weight into the group, and were evenly distributed to 4 experimental groups, with 8 animals in each experimental group. The administration was started on the day of grouping, and the specific administration scheme is shown in the following Table 14:

TABLE 13

| | | Animal | | | | |
|---|---|---|---|---|---|---|
| | | Numbers | Dosage | Administration | Administration | Administration |
| Group | Sample | (mouse) | (mg/kg)^{a} | route | Frequency^{b} | Times |
| G1 | Solvent | 8 | — | i.p. | Q3D | 6 |
| | control PBS | | | | | |
| G2 | Rituximab | 8 | 0.1 | i.v. | Q2W | 2 |
| G3 | QP32700279 | 8 | 10 | i.p. | Q3D | 6 |
| G4 | QP32700279 + | 8 | 10 + 0.1 | i.p. + i.v. | Q3D + Q2W | 6 + 2 |
| | Rituximab | | | | | |

Note:
^{a}The administration volume is calculated as 10 μL/g according to the body weight of the experimental animal.
^{b}Q3D refers to administration once every 3 days, and Q2W refers to administration once every 2 weeks.

The day of grouping and administrating is defined as D0, up to D18, the tumor growth curve reflected by tumor imaging signal values of each group and imaging signal intensity data on D18 are shown in FIG. 12 and Table 15:

TABLE 14

| | | D18 | p value |
|---|---|---|---|
| Group | D18 Signal intensity P/S/cm²/sr | TGI % | v.s. G1 |
| G1 | 3.28E9/5.46E8/D18 | — | — |
| G2 | 1.36E9 ± 3.49E8/18 | 58.6% | 0.0027. |
| G3 | 1.76E9/3.72E8/D18 | 46.4% | 0.0167. |
| G4 | 5.08E8/1.6E8/D18 | 84.5% | <0.0001 |

The results of tumor growth curve show that the groups of Rituximab, QP32700279 and the combination of QP32700279 and Rituximab all significantly inhibit the growth of Raji-Luc tumors, and the tumor growth inhibition rate (TGI) is 58.6%, 46.4% and 84.5%, respectively, and the combination group shows stronger anti-tumor activity than the single-drug groups.

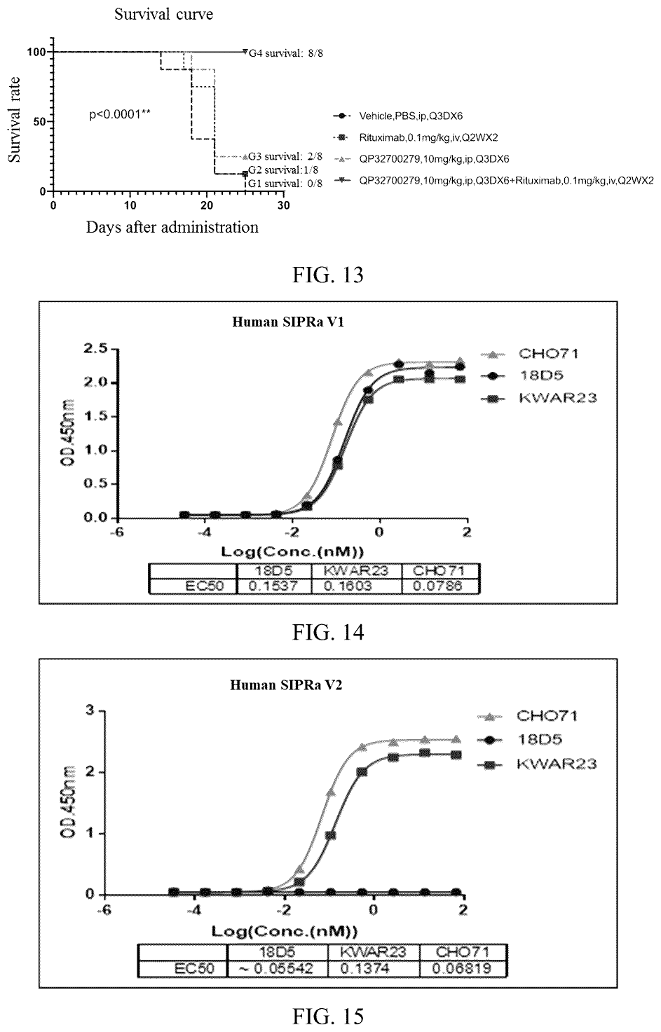

Due to the characteristics of the model, the mice would have abnormal action or paralysis in the later stage of the experiment. At this time, the mice would be euthanized and the survival curve would be recorded. By the end of all mice in G1 group died (D25), the survival curve of each group is shown in FIG. 13.

Kaplan-Meier method was used for survival analysis, Log rank test was used for inter-group comparison, p<0.05 is regarded as significant difference. Compared with the control group, both the QP32700279 group and the combined administration group (QP32700279+Rituximab) could significantly prolong the survival of Raji-Luc tumor-bearing mice (p=0.0445*, p<0.001**), while the Rituximab group could not effectively prolong the survival of tumor-bearing mice (p=0.23). The results suggest that QP32700279 and the combination of QP32700279 and Rituximab can effectively inhibit the tumor growth of Raji-Luc tumor-bearing mice and improve the survival of mice.

### Example 8: ELISA Detection of Anti-SIRPα Antibody Binding to all Subtypes of Human SIRPα

According to the SIRPα V1/V2/V4/V5/V6/V7/V8/V9/V10 sequences reported by the prior literature (“Velcro” Engineering of High Affinity CD47 Ectodomain as Signal Regulatory Protein (SIRP alpha) Antagonists That Enhance Antibody-dependent Cellular Phagocytosis, JOURNAL OF BIOLOGICAL CHEMISTRY, VOLUME 290•NUMBER 20•May 15, 2015), the Fc (mouse IgG2a) of mouse IgG2a subtype was fused to the C-terminal of SIRPα above through gene synthesis and constructed into eukaryotic expression vector pQD. The supernatant of 293E transient transfection at the fifth day was purified by Protein A to obtain SIRPα V1/V2/V3/V4/V5/V6/V7/V8/V9/V10 fusion Fc (mouse IgG2a) proteins respectively. ELISA was further performed to detect binding of SIRPα antibodies to all subtypes of SIRPα. The sequences are shown below.

>SIRPα V1
(SEQ ID NO: 51)
GVAGEEELQVIQPDKSVLVAAGETATLRCTATSLIPVGPIQWFRGAGPGRELIYNQKEGHF
PRVTTVSDLTKRNNMDFSIRIGNITPADAGTYYCVKFRKGSPDDVEFKSGAGTELSVRAK
PS
>SIRPα V2
(SEQ ID NO: 52)
GVAGEEELQVIQPDKSVSVAAGESAILHCTVTSLIPVGPIQWFRGAGPARELIYNQKEGHF
PRVTTVSESTKRENMDFSISISNITPADAGTYYCVKFRKGSPDTEFKSGAGTELSVRAKPS
>SIRPα V3
(SEQ ID NO: 53)
GVAGEEELQVIQPDKSVSVAAGESAILLCTVTSLIPVGPIQWFRGAGPARELIYNQKEGHF
PRVTTVSESTKRENMDFSISISNITPADAGTYYCVKFRKGSPD-TEFKSGAGTELSVRAKPS
>SIRPα V4
(SEQ ID NO: 54)
GVAGEEGLQVIQPDKSVSVAAGESAILHCTATSLIPVGPIQWFRGAGPGRELIYNQKEGHF
PRVTTVSDLTKRNNMDFSIRIGNITPADAGTYYCVKFRKGSPDDVEFKSGAGTELSVRAK
PS
>SIRPα V5
(SEQ ID NO: 55)
GVAGEEELQVIQPDKFVLVAAGETATLRCTATSLIPVGPIQWFRGAGPGRELIYNQKEGHF
PRVTTVSDLTKRNNMDFSIRIGNITPADAGTYYCVKFRKGSPDDVEFKSGAGTELSVRAK
PS
>SIRPα V6
(SEQ ID NO: 56)
GVAGEEELQVIQPDKSVLVAAGETATLRCTATSLIPVGPIQWFRGAGPGRELIYNQKEGHF
PRVTTVSDLTKRNNMDFPIRIGNITPADAGTYYCVKFRKGSPDDVEFKSGAGTELSVRAK
PS
>SIRPα V7
(SEQ ID NO: 57)
GVAGEEELQVIQPDKSVSVAAGESAILHCTVTSLIPVGPIQWFRGAGPARELIYNQKEGHF
PRVTTVSESTKRENMDFSISISNITPADAGTYYCVKFRKGSPD-TEFKSGAGTELSVRGKPS
>SIRPα V8
(SEQ ID NO: 58)
GVAGEEELQVIQPDKSVLVAAGETATLRCTATSLIPVGPIQWFRGAGPARELIYNQKEGHF
PRVTTVSESTKRENMDFSISISNITPADAGTYYCVKFRKGSPD-TEFKSGAGTELSVRAKPS
>SIRPα V9
(SEQ ID NO: 59)
GVAGEEELQVIQPDKSVLVAAGETATLRCTATSLIPVGPIQWFRGAGPGRELIYNQKEGHF
PRVTTVSDLTKRNNMDFSIRISNITPADAGTYYCVKFRKGSPDDVEFKSGAGTELSVRAK
PS
>SIRPα V10
(SEQ ID NO: 60)
RVAGEEELQVIQPDKSVSVAAGESAILHCTVTSLIPVGPIQWFRGAGPARELIYNQKEGHF
PRVTTVSESTKRENMDFSISISNITPADAGTYYCVKFRKGSPD-TEFKSGAGTELSVRAKPS
>FC(mouse IgG2a)
(SEQ ID NO: 61)
EPRGPTIKPCPPCKCPAPNLLGGPSVFIFPPKIKDVLMISLSPIVTCVVVDVSEDDPDVQIS
WFVNNVEVHTAQTQTHREDYNSTLRVVSALPIQHQDWMSGKEFKCKVNNKDLPAPIER
TISKPKGSVRAPQVYVLPPPEEEMTKKQVTLTCMVTDFMPEDIYVEWTNNGKTELNYKN
TEPVLDSDGSYFMYSKLRVEKKNWVERNSYSCSVVHEGLHNHHTTKSFSRTPGK.

### SIRPα Antibody to be Tested:

The SIRPα antibody QP256279 was stably expressed in CHOS cells, and the CHOS stable expression protein was numbered CHO71.

According to the sequence provided by WO2017178653, molecular cloning construction, expression and purification of anti-SIRPα antibody 18D5 of OSE Company were performed for experimental control. Meanwhile, as mentioned above, the QP026249 is the anti-SIRPα antibody KWAR23 of Forty Seven Company, which is expressed here as KWAR23.

Experimental steps of ELISA detection of SIRPα antibody binding to SIRPα V1/V2/V3/V4/V5/V6/V7/V8/V9/V10:

The plate was coated with SIRPα V1/V2/V3/V4/V5/V6/V7/V8/V9/V10 at 1 μg/ml, 60 μl/well overnight at 4° C., and washed twice with PBST; blocked with 5% non-fat milk (Sangon) of 200 μl/well, incubated at room temperature for 1 h, and washed twice with PBST. Antibody was incubated at 10 μg/ml, diluted with 5 times for 10 gradients, 60 μl/well, incubated at room temperature for 1 h, and washed with PBST 5 times. Secondary antibody incubation: anti-hFab 1:10000, 60 μl/well, incubated at room temperature for 1 h, washed with PBST 5 times. Developing: TMB was equilibrated at room temperature for 1 h in advance, 100 μl/well, to develop for 10 min, 2M H₂SO₄ of 50 μl/well was used for termination, and reading was performed at 450 nm on microplate reader.

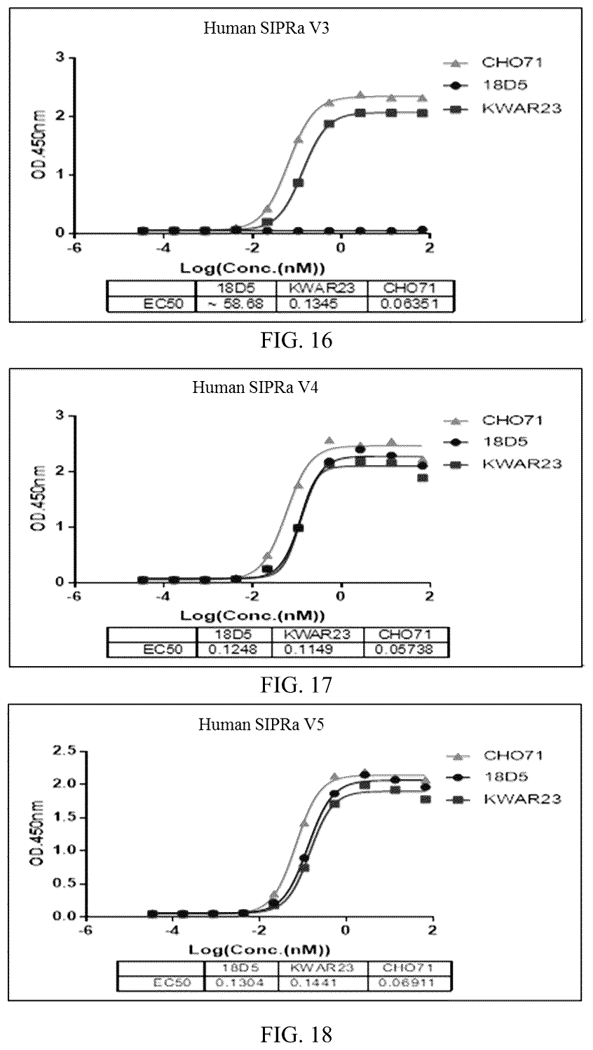

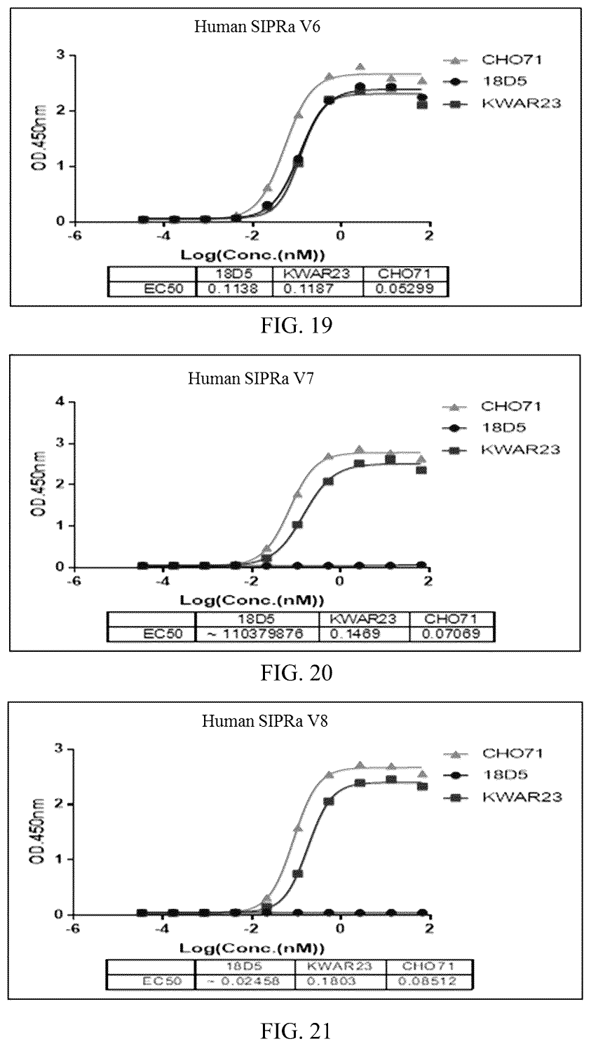

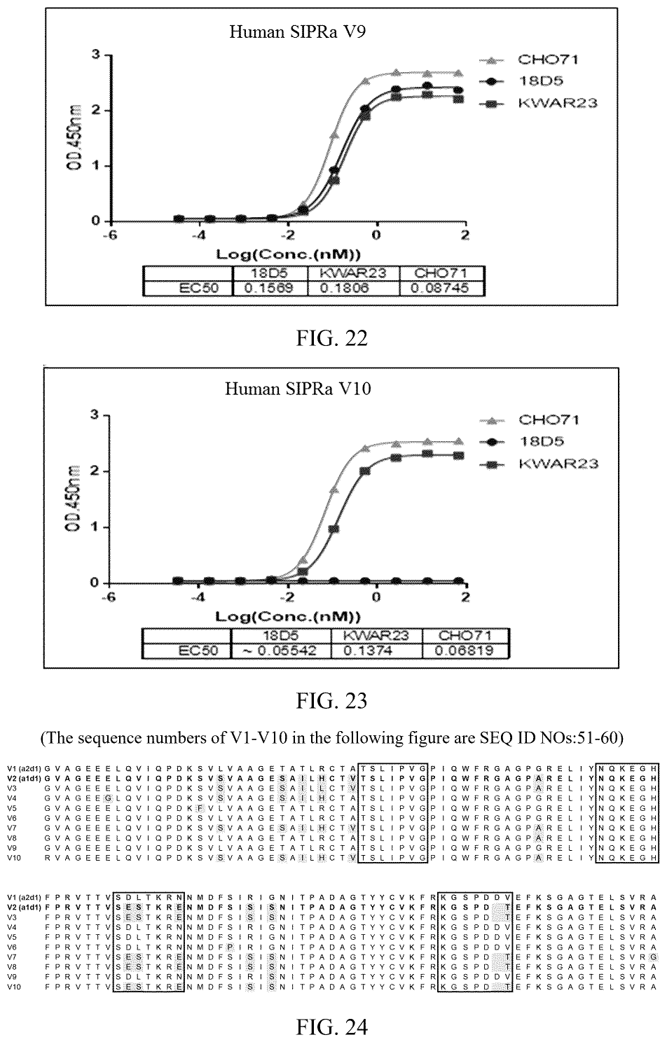

The experimental results are shown in FIGS. 14 to 23, the SIRPα antibody CHO71 of the present invention binds to all subtypes of SIRPα V1/V2/V3/V4/V5/V6/V7/V8/V9/V10. SIRPα antibody 18D5 from OSE does not bind to SIRPα V2/V3/V7/V8/V10.

### Construction and Detection of Bispecific Antibody Targeting SIRPα and PD-L1

Based on the above results, bispecific antibodies targeting SIRPα and PD-L1 were constructed using the sequence of the anti-SIRP a antibody with protein number QP256279. the SIRPα binding domain of the bispecific antibody comprises a heavy chain variable region and a light chain variable region, and the sequence of the heavy chain variable region is selected from QD256, and the sequence of the light chain variable region is selected from QD279. Bispecific antibodies targeting SIRPα and PD-L1 were constructed by combining the sequence of PD-L1 nanobody obtained by the applicant in the previous stage (invention name: An anti-PD-L1 nanobody and use thereof, patent publication number: application number: 202011309419.7); The selected PD-L1 nanobody, with plasmid number QD509, was obtained by immunizing alpaca and humanization. In this study, QD509 can represent the VHH fragment against PD-L1 or the fusion protein of VHH fragment and FC. The amino acid sequence of the VHH fragment is as shown in SEQ ID NO: 62, specifically as follows:

EVQLLESGGGLVQPGGSLRLSCAASGFTLDDYAIGWFRQAPGKGREGV
SCISKSGETTFFVESVKDRFTISRDNSKNTLYLQMNSLRAEDTAVYYC
ATGSWCTVGSMSRQFYRQFFHSWGQGTLVTVSS.

Among them, the sequence order is FR1-CDR1-FR2-CDR2-FR3-CDR3-FR4, and the bolded and underlined parts are CDR1, CDR2, and CDR3, respectively. The amino acid sequences are shown in SEQ ID NO: 63, 64, and 65, respectively.

### Example 9: Cloning Designs

The anti SIRPα/PD-L1 bispecific antibody molecules were designed, by fusing the anti-PD-L1 nanobody QP509 VHH to the C-terminus (QD3282 in Table 16) or N-terminus (QD626 in Table 16) of the SIRPα antibody heavy chain, or to the N-terminus (QD623 in Table 16) of the SIRP a antibody light chain through G4S linker sequences with different numbers of repeats. Primers were designed based on the sequences, and PCR was used to construct the full-length genes of each bispecific antibody. Homologous recombination was performed with the expression vector pQD, and the constructed expression vectors pQD were labeled with plasmid numbers as shown in Table 16. The obtained antibody molecules were then labeled with protein numbers. In Table 16, the amino acid sequences of QD623, QD624, and QD625 are basically similar, with the difference being the number of G4S repeats. Similarly, the amino acid sequences of QD626, QD627, and QD628 are basically similar, with the difference being the number of G4S repeats. The sequence and protein expression numbers of bispecific antibodies are as follows:

TABLE 15
Sequence numbers and protein expression numbers of anti SIRPα/PD-L1 bispecific antibodies

| Protein | Plasmid | Sequence | |
|---|---|---|---|
| number | number | number | Description |
| QP32820279 | QD3282 | SEQ ID | pQDH(hIgG1)-antiSIRPα(QD256)VH-CH1- |
| | | NO: 66 | FC(L234A, L235A, K338A)-(G4S)4-antiPDL1(QD509)VHH |
| | QD279 | SEQ ID | pQDK-(QD279)VL-CL |
| | | NO: 67 | |
| QP32700623 | QD3270 | SEQ ID | pQDH(hIgG1)-antiSIRPα(QD256)VH-CH1- |
| | | NO: 26 | FC(L234A, L235A, K338A) |
| | QD623 | SEQ ID | pQDK-antiPDL1(QD509)VHH-G4S-antiSIRPα(QD279)VL-CL |
| | | NO: 68 | |
| QP32700624 | QD3270 | SEQ ID | pQDH(hIgG1)-antiSIRPα(QD256)VH-CH1- |
| | | NO: 26 | FC(L234A, L235A, K338A) |
| | QD624 | SEQ ID | pQDK-antiPDL1(QD509)VHH-(G4S)2-antiSIRPα(QD279)VL-CL |
| | | NO: 82 | |
| QP32700625 | QD3270 | SEQ ID | pQDH(hIgG1)-antiSIRPα(QD256)VH-CH1- |
| | | NO: 26 | FC(L234A, L235A, K338A) |
| | QD625 | SEQ ID | pQDK-antiPDL1(QD509)VHH-(G4S)3-antiSIRPα(QD279)VL-CL |
| | | NO: 83 | |
| QP06260279 | QD626 | SEQ ID | pQDH-antiPDL1(QD509)VHH-G4S-antiSIRPα(QD256)VH-CH1-FC(L234A, |
| | | NO: 69 | L235A, K338A) |
| | QD279 | SEQ ID | pQDK-(QD279)VL-CL |
| | | NO: 67 | |
| QP06270279 | QD627 | SEQ ID | pQDH-antiPDL1(QD509)VHH-(G4S)2-antiSIRPα(QD256)VH-CH1-FC(L234A, |
| | | NO: 84 | L235A, K338A) |
| | QD279 | SEQ ID | pQDK-(QD279)VL-CL |
| | | NO: 67 | |
| QP06280279 | QD628 | SEQ ID | pQDH-antiPDL1(QD509)VHH-(G4S)3-antiSIRPα(QD256)VH-CH1-FC(L234A, |
| | | NO: 85 | L235A, K338A) |
| | QD279 | SEQ ID | pQDK-(QD279)VL-CL |
| | | NO: 67 | |

In Table 16, the specific sequences of SEQ ID NO: 66 and SEQ ID NO: 67 consisting of QP32820279 are as follows:

>QD3282
(SEQ ID NO: 66)
QVQLVQSGAEVKKPGASVKVSCKASGYTFTNYWINWVRQAPGQGLEWIAMIDPSD
SETHYNQIFKDRATLTTDTSTSTAYMELRSLRSDDTAVYYCAMDYGSLYAMDYWGRGTL
VTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPEA
AGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISAAKGQPREPQVYTLP
PSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGGGGGSGGGGSGGGGSGGGGSEVQL
LESGGGLVQPGGSLRLSCAASGFTLDDYAIGWFRQAPGKGREGVSCISKSGETTFFVESV
KDRFTISRDNSKNTLYLQMNSLRAEDTAVYYCATGSWCTVGSMSRQFYRQFFHSWGQG
TLVTVSS*.
>QD279
(SEQ ID NO: 67)
DIVLTQSPDSLAVSLGERATINCRASQSVRSSGYNWIFWYQQKPGQPPKLLIYLASNR
DSGVPDRFSGSGSGTDFTLTISSLQAEDVAVYYCQHSRELPTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC.

Monoclonal antibodies against SIRPα and fusion FC molecules of anti-PD-L1 nanobodies were designed as controls. The protein number of the monoclonal antibody against SIRPα is QP32700279, and the molecular numbers of fusion FC molecules of anti-PD-L1 nanobodies are QP509 and QP3447, as shown in Table 17:

TABLE 16

| | Plasmid | Sequence | |
|---|---|---|---|
| Protein number | number | number | Description |
| QP32700279 | QD3270 | SEQIDNO: 26 | pQDH(hIgG1)-antiSIRPα(QD256)VH-CH1- |
| | | | FC(L234A, L235A, K338A) |
| | QD279 | SEQIDNO: 67 | pQDK-(QD279)VL-CL |
| QP3447 | QD3447 | SEQIDNO: 70 | pQD-FC-PD-L1VHH |
| QP509 | QD509 | SEQIDNO: 71 | pQD-PD-L1VHH-FC |

Additionally, based on existing literature, anti-SIRPα antibodies and anti-PD-L1 antibodies were designed as controls. They are specifically shown in Table 18. Among them, QP026249 is a SIRPα monoclonal antibody KWAR23 analogue from 47 company. QP250251 is an analogue of the anti-SIRPα monoclonal antibody 18D5 from OSE Immunotherapeutics. QP37503751 is an analog of the anti-SIRPα monoclonal antibody 1H9 from 47 company. QP11801181 is an analog of Tecentriq, which is the anti-PD-L1 monoclonal antibody Atezolizumab from Roche.

TABLE 17

| | Plasmid | | |
|---|---|---|---|
| Protein number | number | Sequence number | Description |
| QP026249 | QD026 | SEQIDNO: 10 | pQDK-KWAR23-L |
| | QD249 | SEQIDNO: 12 | pQDH-KWAR23-H(IgG4) |
| QP250251 | QD250 | SEQIDNO: 72 | pQD-18D5VL-CL |
| | QD251 | SEQIDNO: 73 | pQD-18D5VH-CH1-FC |
| QP37503751 | QD3750 | SEQIDNO: 74 | QD3750-pQDH-hz1H9.hIgG1(N297A).gb |
| | QD3751 | SEQIDNO: 75 | QD3751-pQDK-hz1H9(antiSirpα).gb |
| QP11801181 | QD1181 | SEQIDNO: 76 | pQD-3280AVL-CL |
| | QD1180 | SEQIDNO: 77 | pQD-3280AVH-CH1-FC |

Based on existing literature, SIRPα antigens were designed for the experiment, as shown in Table 19:

TABLE 18

| Protein | Plasmid | Sequence | |
|---|---|---|---|
| number | number | number | Description |
| QP001 | QD001 | SEQIDNO: 78 | pQD-CD47-FC |
| QP002 | QD002 | SEQIDNO: 79 | pQD-SIRPα-FC |
| QP093 | QD093 | SEQIDNO: 80 | pQD-humanSIRPαV1ECD(E31-R370)-mFC(IgG2a) |
| QP095 | QD095 | SEQIDNO: 81 | pQD-humanSIRPαV2ECD(E31-R370)-mFC(IgG2a) |
| QP094 | QD094 | SEQIDNO: 45 | pQD-humanSIRPαV1ECD(E31-R370)-flag-his |
| QP096 | QD096 | SEQIDNO: 46 | pQD-humanSIRPαV2ECD(E31-R370)-flag-his |
| QP098 | QD098 | SEQIDNO: 47 | pQD-MACFASIRPα(I7G9Z7)ECD(E31-R369)-flag-his |
| QP271 | QD271 | SEQIDNO: 49 | pQD-monkeySIRPα1-flag-hisT-557-28 |
| QP532 | QD532 | SEQIDNO: 53 | pQD-SIRPαV3-mFC(mIgG2a) |
| QP533 | QD533 | SEQIDNO: 54 | pQD-SIRPαV4-mFC(mIgG2a) |
| QP534 | QD534 | SEQIDNO: 55 | pQD-SIRPαV5-mFC(mIgG2a) |
| QP535 | QD535 | SEQIDNO: 56 | pQD-SIRPαV6-mFC(mIgG2a) |
| QP536 | QD536 | SEQIDNO: 57 | pQD-SIRPαV7-mFC(mIgG2a) |
| QP537 | QD537 | SEQIDNO: 58 | pQD-SIRPαV8-mFC(mIgG2a) |
| QP538 | QD538 | SEQIDNO: 59 | pQD-SIRPαV9-mFC(mIgG2a) |

Note:
QP098 is the sequence of cynomolgus monkey SIRPα (Uniprot database sequence number I7G9Z7); QP271 is the sequence of rhesus monkey SIRPα, obtained by the inventor through sequencing monkey PBMC. QP532~QP538 provide sequences for encoding SIRPα molecules, and the mFC sequence is shown in SEQ ID NO: 86.

### Example 10: Protein Expression and Purification

The proteins in Example 9 were subject to expression and purification, referring to the expression and purification method of proteins in Example 2. Each antibody was further subject to SEC purification. The results showed that the transient expression yields of the anti-SIRPα/PD-L1 bispecific antibodies were good, with good SEC purity and stable physicochemical properties.

### Example 11: SPR Detection of SIRPα/PD-L1 Bispecific Antibody Binding Affinity to SIRPα/PD-L1

(1) This study used Biacore8K to detect the affinity between antibody molecules and the antigen SIRPα. As described above, QP026249 is a SIRPα monoclonal antibody KWAR23 analogue from 47 company. The results are shown in the table below:

TABLE 19
Biacore detection of the binding affinity
of antibody molecules to human SIRPα V1

| Antibody | Antigen | kₐ(1/Ms) | k_{d}(1/s) | KD (M) |
|---|---|---|---|---|
| QP32700623 | SIRPαV1-his (QP094) | 7.17E+05 | 1.35E−04 | 1.88E−10 |
| QP32700624 | SIRPαV1-his (QP094) | 4.02E+05 | 9.78E−05 | 2.43E−10 |
| QP32700625 | SIRPαV1-his (QP094) | 4.00E+05 | 1.25E−04 | 3.13E−10 |
| QP06260279 | SIRPαV1-his (QP094) | 9.74E+05 | 1.51E−04 | 1.55E−10 |
| QP06270279 | SIRPαV1-his (QP094) | 1.08E+06 | 1.35E−04 | 1.25E−10 |
| QP06280279 | SIRPαV1-his (QP094) | 1.48E+06 | 1.68E−04 | 1.14E−10 |
| QP32820279 | SIRPαV1-his (QP094) | 3.05E+06 | 1.83E−04 | 6.00E−11 |
| QP026249 | SIRPαV1-his (QP094) | 8.22E+05 | 3.86E−03 | 4.70E−09 |
| (Control) | | | | |

The results showed that the different forms of bispecific antibody molecules designed by the present invention all had high affinity for binding to the SIRPα V1 recombinant protein.
(2) Then the affinity between antibody molecules and the antigen PD-L1 was detected by Biacore8K. QP11801181 is an analog of the PD-L1 monoclonal antibody Tecentriq from Roche company. QP509 represents the fusion of PD-L1 nanobody VHH at the N-terminus of FC, while QP3447 represents the fusion of PD-L1 nanobody at the C-terminus of FC. The results are shown in the table below:

TABLE 20
Biacore detection of the binding affinity
of antibody molecules to human PD-L1

| Antibody | Antigen | ka (1/Ms) | kd (1/s) | KD (M) |
|---|---|---|---|---|
| QP32820279 | PD-L1-his (QPP09) | 1.01E+06 | 4.33E−04 | 4.29E−10 |
| QP32700624 | PD-L1-his (QPP09) | 7.44E+05 | 1.22E−03 | 1.64E−09 |
| QP32700625 | PD-L1-his (QPP09) | 8.01E+05 | 1.03E−03 | 1.29E−09 |
| QP3447 | PD-L1-his (QPP09) | 8.58E+05 | 4.11E−04 | 4.79E−10 |
| (Control) | | | | |
| QP509 | PD-L1-his (QPP09) | 1.65E+06 | 4.30E−04 | 2.61E−10 |
| (Control) | | | | |
| QP11801181 | PD-L1-his (QPP09) | 2.07E+05 | 2.87E−04 | 1.39E−09 |
| (Control) | | | | |

The results showed that the different forms of bispecific antibody molecules designed by the present invention all had high affinity for binding to the human PD-L1 recombinant protein, and the affinity of PD-L1 nanobody VHH fused to the C-terminus of FC was slightly higher than that fused to the N-terminus.

### Example 12: ELISA Detection of SIRPα/IPD-L1 Bispecific Antibody Binding to PD-L1 and SIRPα Protein

### (1) ELISA Detection of Antibodies Binding to PD-L1:

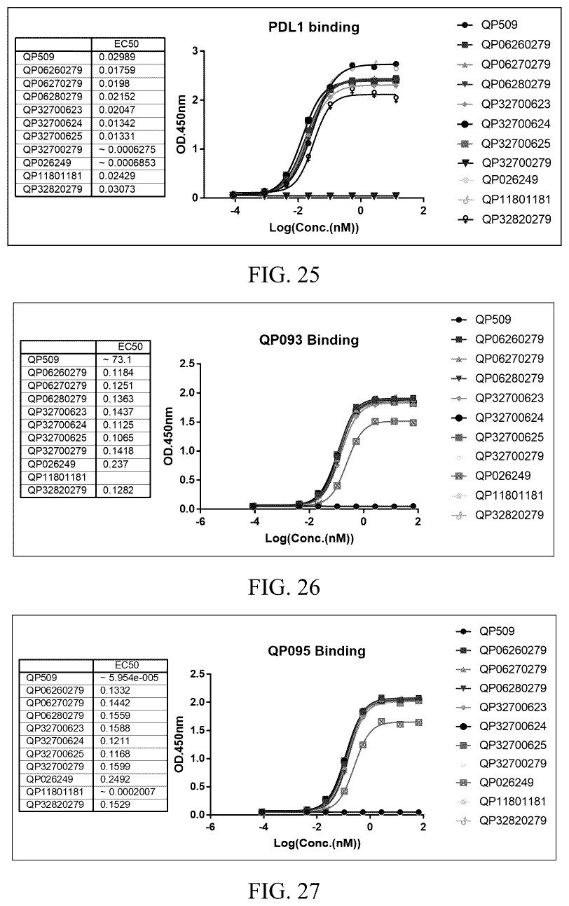

Plate coating: anti-his, 1 μg/mL in PBS, 60 μL/well, 4° C. overnight, washed twice with PBST. Blocking: 5% non-fatmilk (Sangon), 200 μl/well, 25° C. 120 rpm, being subjected to incubation for 1 hour. The antigen QP003 (PDL1-his) was incubated at 1 μg/ml, 60 μl/well, 25° C. 120 rpm, 1 h, then the plate was washed with PBST for 5 times. The primary antibody was incubated starting from 13.3 nM, diluted 5 times for 7 gradients, and the last one was 10-fold dilution, 60 μl/well, 25° C. 120 rpm, 1 h, then the plate was washed with PBST for 5 times. The secondary antibody anti-hFc (1:5000) was incubated at 60 μl/well, 25° C. 120 rpm, 1 hour, then the plate was washed with PBST for 5 times. Developing: TMB was equilibrated at room temperature for 1 hour in advance, and the development was performed for 3 minutes. Termination: 1M H₂SO₄ was used to terminate the development. The results are shown in FIG. 25.

### (2) ELISA Detection of Antibody Molecules Binding to SIRPα:

Plate coating: plates were coated with QP093 (SIRPαV1) and QP095 (SIRPαV2), respectively, 1 μg/mL in PBS, 60 μL/well, 4° C. overnight, washed twice with PBST. Blocking: 5% non-fatmilk (Sangon), 200 μl/well, 25° C. 120 rpm, being subjected to incubation for 1 hour; The primary antibody was incubated starting from 66.7 nM, diluted 5 times for 7 gradients, and the last one was 50-fold dilution, 60 μl/well, 25° C. 120 rpm, 1 h, then the plate was washed with PBST for 5 times; The secondary antibody anti-hFab (without glycerol, 1:10000) was incubated at 60 μl/well, 25° C. 120 rpm, 1 hour, then the plate was washed with PBST for 5 times; Developing: TMB was equilibrated at room temperature for 1 hour in advance, and the development was performed for 3 minutes; Termination: 1M H₂SO₄ was used to terminate the development. The results are shown in FIG. 26 and FIG. 27

### Example 13: Detection of Antibody Molecules Blocking the Binding of Human PD-L1 to PD-1 Protein

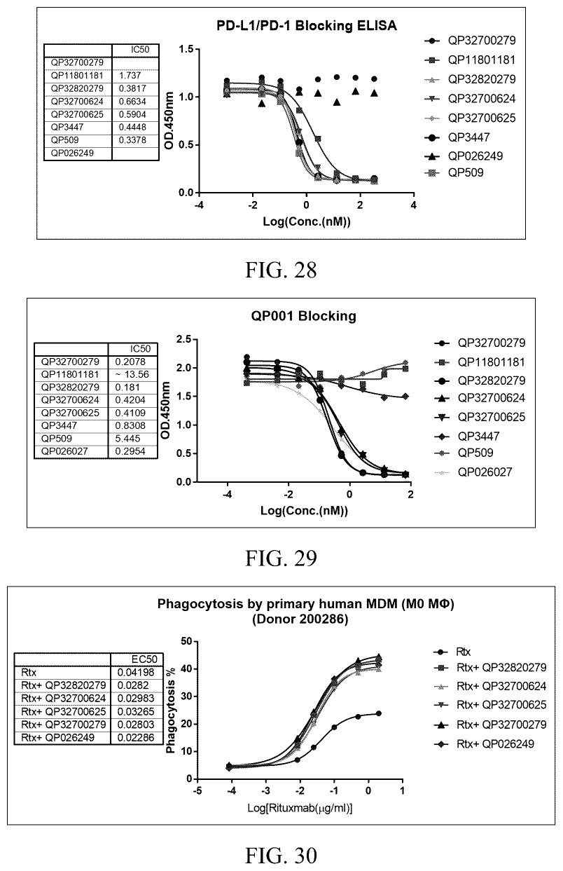

The plate was coated with protein QP1138 (PD1-FC) at 2 μg/ml, 50 μl/well, overnight at 4° C. Then the plate was washed with PBS for 3 times. Blocking: 3% BSA 250 μl/well, being subjected to incubation at room temperature for 1 hour. 2 μg/ml PDL1-mouse FC and different concentrations of antibodies were prepared, mixed evenly in equal volumes, and subjected to incubation at room temperature for 1 hour. The plate was washed with PBST for 3 times, and washed with PBS for 3 times. The secondary antibody HRP-mouse IgG (1:5000) was incubated at 50 μl/well, then the plate was washed with PBST for 6 times and with PBS for 3 times. Developing: TMB 100 μl/well, developing for 3 minutes. 2M H₂SO₄ 50 μl/well for termination. The results are shown in FIG. 28. The bispecific antibody molecules could block the binding of human PD-L1 to PD-1 protein.

### Example 14: Detection of Antibody Molecules Blocking the Binding of Human CD47 to SIRPα Protein

Blocking-ELISA experimental method: the plate was coated with CD47 FC (QP001) at 2 g/ml, overnight at 4° C., washed with PBS for 3 times, blocked with 5% milk 250 μl/well, incubated with a mixture of Biotin SIRPα-FC (QP002) 0.05 μg/ml+Abs 50 μg/ml at a ratio of 1:1 at 25° C. for 1 hour, then incubated with HRP-Strepavin (1:5000). The results are shown in FIG. 29.

### Example 15: Synergistically Enhancing CD20 Antibody Rituxan Dependent ADCP

Antibody dependent macrophage mediated phagocytosis (ADCP) refers to the binding of the Fab segment of an antibody to the antigen epitope of tumor cells, and its Fc segment binding to the FcγR on the surface of macrophages, mediating macrophage phagocytosis of target cells. At the same time, SIRPα expressed on macrophages binds to CD47 expressed on tumor cells to form inhibitory signals. Further study was conducted through ADCP to investigate biological activity of bispecific antibody molecules blocking the binding of macrophage SIRPα to CD47 in Raji cells, thereby synergistically enhancing the CD20 antibody Rituxan dependent macrophage phagocytosis of human Burkitt's lymphoma cell Raji.

Monocytes were separated from peripheral blood mononuclear cells (PBMCs) of healthy individuals, and were added with 50 ng/mL Human Recombinant M-CSF to induce differentiation into macrophages. Raji cells were labeled with green fluorescent CFSE, and were inoculated into a 96 well plate with macrophages at a ratio of 2:1, and then the plate was added with different concentrations of the CD20 antibody Rituxan alone or in combination with SIRPα antibodies. After incubating at 37° C. for 2 hours, the reaction was terminated, and then the APC anti-human CD11b antibody was incubated. The percentage of APC/FITC double positive cells at each concentration of antibody was obtained by FACS reading, which was the percentage of macrophages exhibiting phagocytic behavior.

The results are shown in FIG. 30. The results showed that the combination of the antibodies of the present invention and Rituximab resulted in a smaller EC50 and significantly enhanced ADCP synergistic effect compared to Rituximab alone.

### Example 16: Biological Activity of Bispecific Antibody Molecule Stimulating Human PBMC In Vitro Proliferation

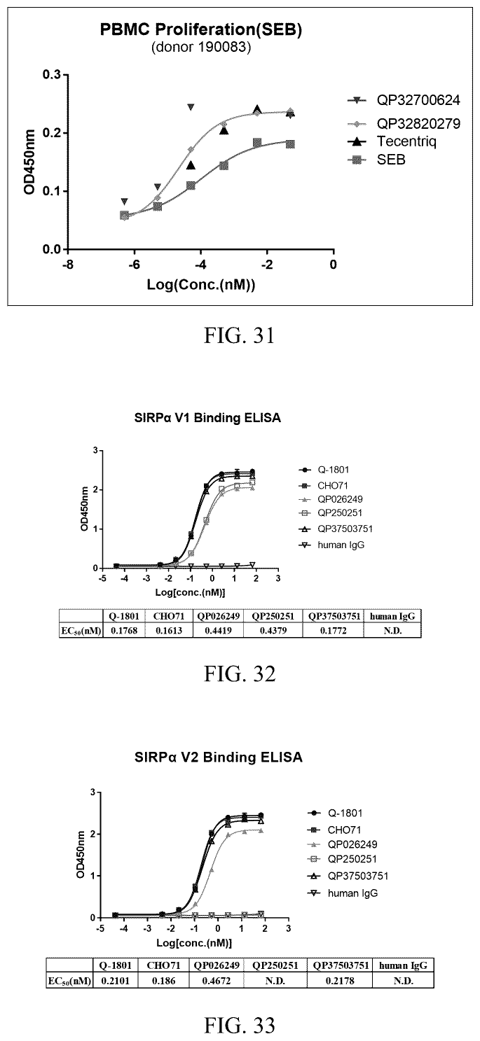

Human peripheral blood mononuclear cells (PBMCs) are composed of various white blood cells, mainly including monocytes, B cells, T cells, NK cells, dendritic cells, and macrophages, etc. Superantigen SEB was added in vitro to stimulates PBMCs, and through the presentation and activation of APC cells, lymphocytes were activated and began to proliferate, producing a variety of cytokines. PD-L1 antibody enhances T cell proliferation and the release of cytokines such as IL-2 by blocking the immunosuppressive signal of PD-1/PD-L1 binding. The biological activity of antibody molecules in PBMC in vitro proliferation assay was further investigated by detecting IL-2 release through ELISA. PBMC cells were inoculated into a 96 well plate, SEB at different concentrations were prepared and added to the wells containing PBMC cells. Then the antibody molecules of the present invention and other control antibodies were added thereto, mixed gently, and subjected to culture for 2 days. IL-2 secretion in cell culture supernatant was detected by ELISA. The results showed that QP32700624 and QP32820279 significantly enhanced the activation and proliferation of PBMCs, as well as the production of IL-2, in the in vitro proliferation assay of PBMCs stimulated by SEB. It was comparable to the control antibody Tecentriq. The results are shown in FIG. 31.

### Example 17: Q-1801 Protein Expression and Purification

The sequence of the anti-SIRPα/anti-PD-L1 bispecific antibody QP32820279 was transferred into a pCHO vector, which is a laboratory modified vector containing GS as a screening marker and can be used for screening stable transfected CHO cells. The stable transfected CHO cells were subjected to GS pressure screening to obtain a cell line with high expression of anti-SIRPα/anti-PD-L1 bispecific antibodies for protein production. After purification, the target molecule was obtained, designated as CHO44 (named Q-1801). The protein sequence numbers consisting of CHO44 were the same as QP32820279 (with the amino acid sequence numbers SEQ ID NO: 66, SEQ ID NO: 67). At the same time, SIRPα monoclonal antibody QP32700279 was also expressed and purified in CHO cells, designated as CHO71, as a SIRPα single component control. The PD-L1 nanobody VHH was fused to the C-terminus of FC, thereby obtaining a protein designated as QP3447, which served as a PD-L1 single component control.

### Example 18: Q-1801 Binding to Human and Monkey SIRPα

### (1) SPR

The gene encoding SIRPα is a polymorphic gene, and 10 variants have been identified in the population. The polymorphism of human SIRPα leads to changes in surface exposed amino acids, but does not affect the binding to CD47. The most common protein variants are SIRPα V1 and V2 (accession numbers NP_542970 (P78324) and CAA71403). This study used Biacore8K to detect the affinity between molecules such as Q-1801 and the antigen SIRPα. The results of the binding affinity of Q-1801 and QP026249 to human SIRPα V1, as well as the binding affinity of Q-1801 to human SIRPα V2, cynomolgus monkey SIRPα, and rhesus monkey SIRPα, are summarized in Table 22. The results showed that Q-1801 had a high affinity for binding to human SIRPα V1, with a KD value of 6.01E-11 (M); binding to SIRPα V2 with a KD value of 1.03E-10 (M); binding to cynomolgus monkey SIRPα with a KD value of 2.14E-09 (M); binding to rhesus monkey SIRPα with a KD value of 5.22E-10 (M). The affinity of Q-1801 binding to human SIRPα V1 was significantly higher than that of KWAR23 analog (QP026249), and the affinity KD value of KWAR23 analog binding to human SIRPα V1 was 4.69E-09M; The affinity of Q-1801 binding to human SIRPα V2 was significantly higher than that of KWAR23 analog (QP026249), and the affinity KD value of KWAR23 analog binding to human SIRPα V2 was 1.75E-08 (M).

TABLE 22
The affinity of molecules such as Q-1801
binding to human and monkey SIRPα proteins

| Antibody | Antigen | KD (M) | kₐ(1/Ms) | k_{d}(1/s) |
|---|---|---|---|---|
| Q-1801 | Human SIRPα V1 | 6.01E−11 | 3.05E+06 | 1.83E−04 |
| | (QP094) | | | |
| QP026249 | Human SIRPα V1 | 4.69E−09 | 8.22E+05 | 3.86E−03 |
| | (QP094) | | | |
| Q-1801 | Human SIRPα V2 | 1.03E−10 | 2.27E+06 | 2.34E−04 |
| | (QP096) | | | |
| QP026249 | Human SIRPα V2 | 1.75E−08 | 1.38E+06 | 2.42E−02 |
| | (QP096) | | | |
| Q-1801 | Cynomolgus monkey | 2.14E−09 | 5.46E+06 | 1.17E−02 |
| | SIRPα | | | |
| Q-1801 | rhesus monkey SIRPα | 5.22E−10 | 5.81E+06 | 3.03E−03 |
| | (QP271) | | | |

### (2) ELISA

SIRPα gene polymorphism exists in different ethnic groups. According to existing literature, there are different proportions of SIRPα V1/V2 in each ethnic group, with East Asians having a SIRPα V2 gene as high as 42.3%. The binding of Q-1801 and other molecules to different subtypes of human SIRPα and mouse SIRPα was detected by ELISA.

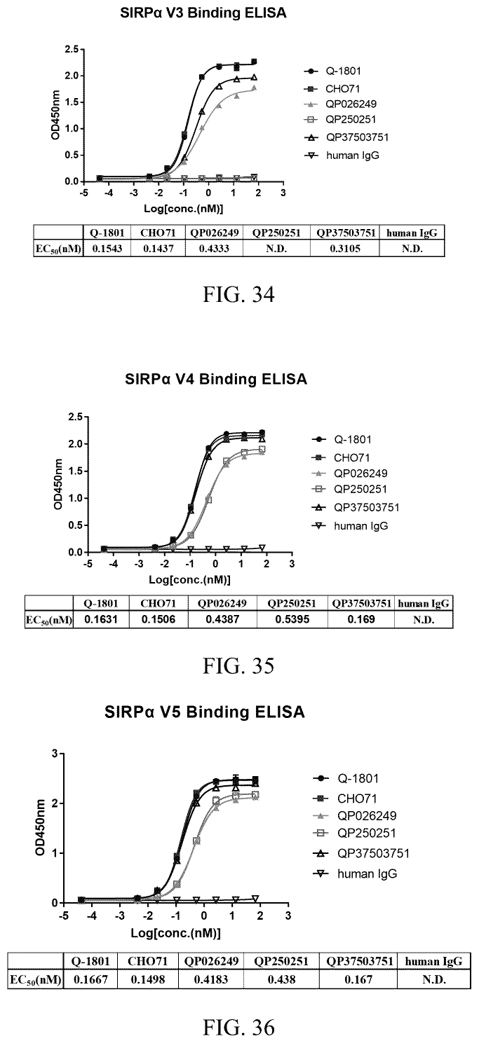

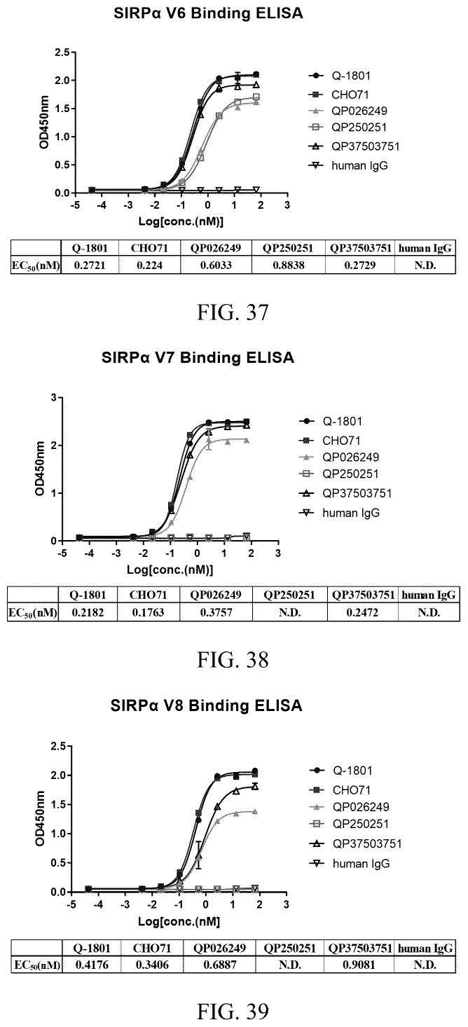

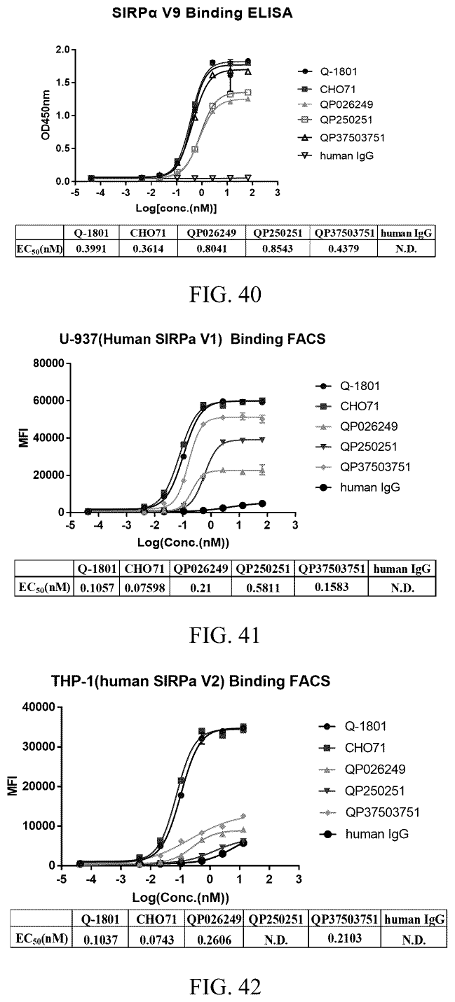

The inventor constructed 9 kinds of reported SIRPα genotypes V1-V9. Human SIRPα V1 recombinant protein (QP093), human SIRPα V2 recombinant protein (QP095), human SIRPα V3 recombinant protein (QP532), human SIRPα V4 recombinant protein (QP533), human SIRPα V5 recombinant protein (QP534), human SIRPα V6 recombinant protein (QP535), human SIRPα V7 recombinant protein (QP536), human SIRPα V8 recombinant protein (QP537), and human SIRPα V9 recombinant protein (QP538) were coated onto an ELISA plate, and then gradient diluted tested molecules such as Q-1801 were added thereto. HRP labeled anti-humanFc secondary antibody was used for detection, and the results are shown in FIG. 32 to FIG. 40: Q-1801 bound to human SIRPα V1, EC50=0.1768 nM; bound to human SIRPα V2, EC50=0.2101 nM; bound to human SIRPα V3, EC50=0.1543 nM; bound to human SIRPα V4, EC50=0.1631 nM; bound to human SIRPα V5, EC50=0.1667 nM; bound to human SIRPα V6, EC50=0.2721 nM; bound to human SIRPα V7, EC50=0.2182 nM; bound to human SIRPα V8, EC50=0.4176 nM; bound to human SIRPα V9, EC50=0.3991 nM. The 18D5 analog did not bind to human SIRPα V2, SIRPα V3, SIRPα V7, and SIRPα V8.

In summary, Q-1801 has a high affinity for all genotypes of SIRPα, while QP250251 (18D5 analog) does not bind to SIRPα V2/V3/V7/V8/V10, and QP026249 (KWAR23 analog) binds weakly to SIRPα V2/V3/V7/V8/V10.

### (3) FACS

It has been reported that the U-937 cell line is a human histiocytic lymphoma cell line expressing endogenous SIRPα V1, and the THP-1 cell line is a human monocytic leukemia cell line expressing endogenous SIRPα V2. The binding of Q-1801 to human SIRPα V1 and human SIRPα V2 proteins was tested by FACS assay. Q-1801 and other molecules at different concentrations were incubated with U-937 cells and THP-1 cells, respectively. The fluorescence values were detected by FACS, fitting curves were drew, and the values of EC50 were compared.

The results are shown in FIG. 41: the EC50 of Q-1801 binding to U-937 (human SIRPα V1) was 0.1057 nM, CHO71 binding EC50 was 0.07598 nM, QP026249 (KWAR23 analog) binding EC50 was 0.21 nM, QP37503751 (1H9 analog) binding EC50 was 0.1583 nM, and QP250251 (18D5 analog) binding EC50 was 0.5811 nM. The affinity of Q-1801 binding to human SIRPα V1 was comparable to that of monoclonal antibody CHO71.

The results are shown in FIG. 42: the EC50 of Q-1801 binding to THP-1 (human SIRPα V2) was 0.1037 nM, CHO71 binding EC50 was 0.0743 nM, QP026249 (KWAR23 analog) binding EC50 was 0.2606 nM, QP37503751 (1H9 analog) binding EC50 was 0.2103 nM, and QP250251 (18D5 analog) did not bind. The affinity of Q-1801 binding to human SIRPα V2 was comparable to that of monoclonal antibody CHO71.

In summary, Q-1801 has high affinity for binding to human SIRPα V1 and human SIRPα V2. QP250251 (18D5 analog) does not bind to human SIRPα V2.

### Example 19: Q-1801 Binding to Human and Monkey PD-L1

### (1) SPR

The affinity between molecules such as Q-1801 and the antigen PD-L1 was detected by Biacore8K. The affinity results of Q-1801, QP3447, QP11801181 (Tecentriq analog) with human PD-L1, and the affinity results of Q-1801 with rhesus monkey PD-L1 are summarized in Table 23.

TABLE 23
The affinity of molecules such as Q-1801 binding
to human PD-L1/rhesus monkey PD-L1

| Antibody | Antigen | KD (M) | kₐ(1/Ms) | k_{d}(1/s) |
|---|---|---|---|---|
| Q-1801 | Human PD-L1 | 4.31E−10 | 1.01E+06 | 4.33E−04 |
| QP3447 (Control) | Human PD-L1 | 4.79E−10 | 8.58E+05 | 4.11E−04 |
| QP11801181 | Human PD-L1 | 1.39E−09 | 2.07E+05 | 2.87E−04 |
| (Control) | | | | |
| Q-1801 | Rhesus PD-L1 | 4.99E−10 | 1.56E+06 | 7.79E−04 |

The results showed that the KD value of Q-1801 with high affinity binding to human PD-L1 was 4.31E-10 (M); The KD value of Q-1801 binding to rhesus monkey PD-L1 was 4.99E-10 (M). The KD value of QP3447 binding to human PD-L1 was 4.79E-10 (M). The KD value of Tecentriq analog binding to human PD-L1 was 1.39E-09 (M).

In summary, both Q-1801 and QP3447 have high affinity for binding to human PD-L1, and their affinities are comparable; The affinity of Q-1801 binding to human PD-L1 is higher than that of PD-L1 positive antibody Tecentriq analog.

### (2) ELISA

The binding of antibody molecules such as Q-1801 to human PD-L1 detected by ELISA.

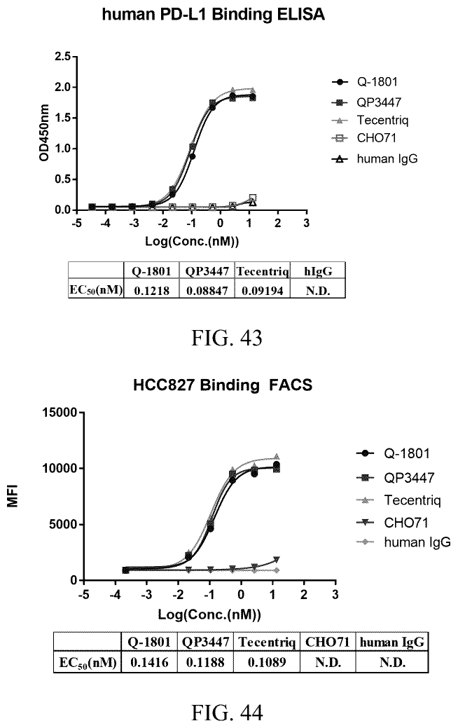

The anti-HIS antibody was coated onto an ELISA plate, and different species of PD-L1 proteins were added. After incubation, gradient diluted Q-1801 and other molecules were added. The HRP labeled anti-humanFc secondary antibody was used for detection. The results are shown in FIG. 43: Q-1801 bound to human PD-L1 protein, with an EC50 value of 0.1218 nM. QP3447 bound to human PD-L1 protein, with an EC50 value of 0.08847 nM. Tecentriq bound to human PD-L1, with an EC50 value of 0.09194 nM.

### (3) FACS

It has been reported that the HCC827 cell line is a human lung cancer cell line expressing endogenous PD-L1. The binding of Q-1801 to HCC827 cells naturally expressing human PD-L1 protein was tested by FACS. Q-1801 and other molecules at different concentrations were incubated with HCC827 cells, respectively. The fluorescence values were detected by FACS, fitting curves were drew, and the values of EC50 were compared. The results are shown in FIG. 44: the EC50 of Q-1801 binding to human PD-L1 was 0.1416 nM, the EC50 of QP3447 binding was 0.1188 nM, and the EC50 of Tecentriq binding was 0.1089 nM.

In summary, Q-1801, PD-L1 monoclonal antibody QP3447, and Tecentriq all bind to HCC827 cells naturally expressing human PD-L1, with comparable binding affinity.

### Example 20: Q-1801 Blocking the Binding of SIRPα to CD47

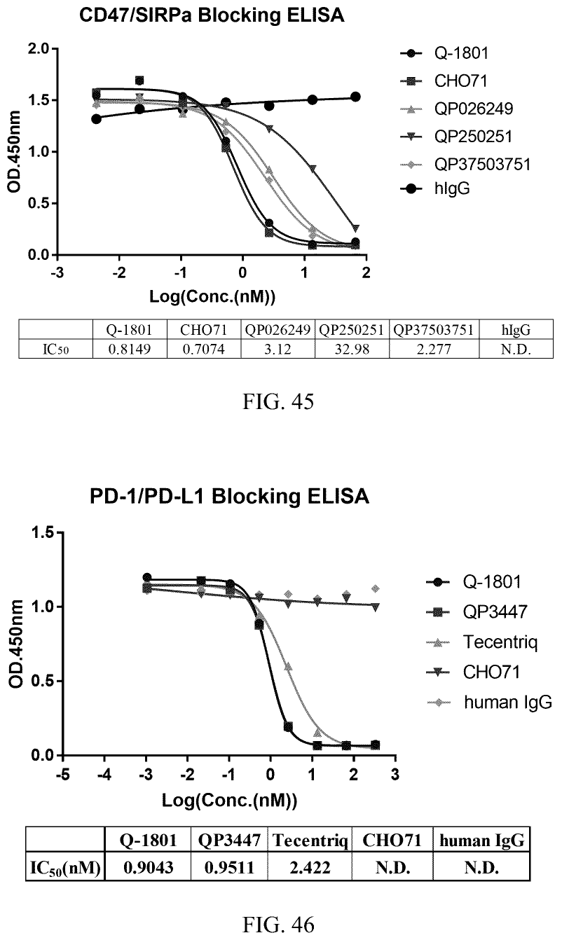

The CD47 protein overexpressed on the surface of tumor cells binds with SIRPα expressed on the surface of macrophages, escaping the phagocytosis of macrophages. The Q-1801 molecule can block the binding of CD47 to SIRPα, causing the loss of the “don't eat me” signal and promoting macrophage attack on tumors. Competitive ELISA was used to detect the ability of Q-1801 and other molecules to block the binding of SIRPα to CD47. The human CD47 protein was coated onto an ELISA plate, and SIRPα-mouse Fc protein was added. After incubation, gradient diluted Q-1801 and other molecules were added. The HRP labeled anti-mouseIgG secondary antibody was used for detection. The results are shown in FIG. 45: The IC50 of Q-1801 blocking the binding of SIRPα to CD47 was 0.8149 nM, the IC50 of CHO71 blocking the binding of SIRPα to CD47 was 0.7074 nM, the IC50 of QP026249 (KWAR23 analog) blocking the binding of SIRPα to CD47 was 3.12 nM, the IC50 of QP37503751 (1H9 analog) binding of SIRPα to CD47 was 2.277 nM, and the IC50 of QP250251 (18D5 analog) blocking the binding SIRPα to CD47 was 32.98 nM.

In summary, the ability of Q-1801 to block the binding of SIRPα to CD47 is comparable to that of SIRPα monoclonal antibody CHO71, and is significantly stronger than that of QP026249 (KWAR23 analog), QP37503751 (1H9 analog), and QP250251 (18D5 analog).

### Example 21: Q-1801 Blocking the Binding of PD-L1/PD-1, PD-L1/CD80

PD-L1 has two ligands, PD-1 and CD80. The C-terminus of the Q-1801 molecule is an anti-PD-L1 nanobody, which blocks both the binding of PD-L1 to PD-1 and the binding of CD80 to PD-L1.

Competitive ELISA was used to detect the ability of Q-1801 and other molecules to block the binding of PD-L1 to PD-1. The human PD-1 protein was coated onto an ELISA plate, and PD-L1-mouse Fc protein was added. After incubation, gradient diluted Q-1801 and other molecules were added. The HRP labeled anti-mouseIgG secondary antibody was used for detection. The results are shown in FIG. 46: the IC50 of Q-1801 blocking PD-L1 and PD-1 was 0.9043 nM, the IC50 of QP3447 blocking PD-L1 and PD-1 was 0.9511 nM, and the IC50 of Tecentriq blocking PD-L1 and PD-1 was 2.422 nM.

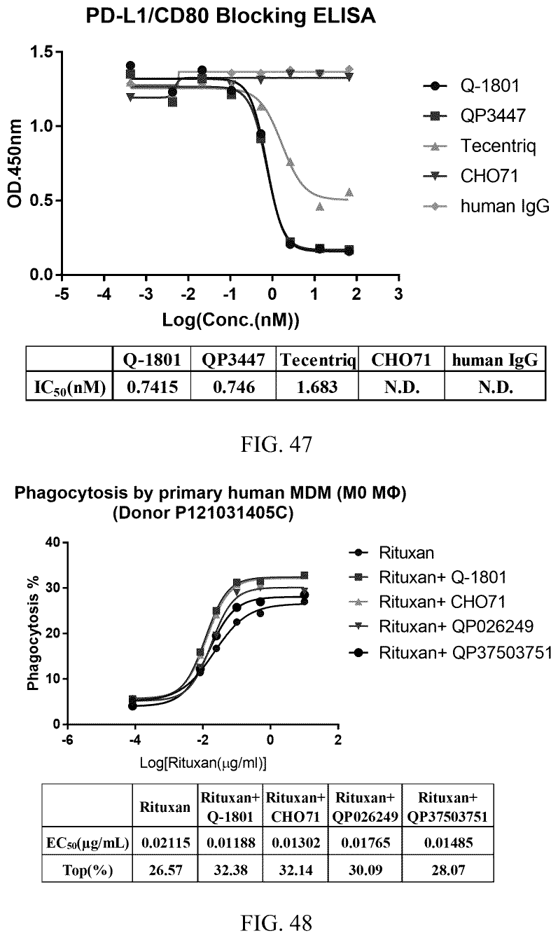

Competitive ELISA was used to detect the ability of Q-1801 and other molecules to block the binding of CD80 to PD-L1. The human CD80 protein was coated onto an ELISA plate, and PD-L1-mouse Fc protein was added. After incubation, gradient diluted Q-1801 and other molecules were added. The HRP labeled anti-mouseIgG secondary antibody was used for detection. The results are shown in FIG. 47: the IC50 of Q-1801 blocking PD-L1 and CD80 was 0.7415 nM, the IC50 of QP3447 blocking PD-L1 and CD80 was 0.746 nM, and the IC50 of Tecentriq blocking PD-L1 and CD80 was 1.683 nM.

In summary, Q-1801 blocks both PD-L1/PD-1 binding and PD-L1/CD80 binding; The ability of Q-1801 to block PD-L1/PD-1 and PD-L1/CD80 binding is superior to Tecentriq.

### Example 22: Q-1801 Synergistically Enhancing CD20 Antibody Rituxan Dependent ADCP

Antibody dependent macrophage mediated phagocytosis (ADCP) refers to the binding of the Fab segment of an antibody to the antigen epitope of tumor cells, and its Fc segment binding to the FcγR on the surface of macrophages, mediating macrophage phagocytosis of target cells. However, SIRPα expressed on macrophages binds to CD47 expressed on tumor cells will form inhibitory signals. Raji cells are human Burkitt lymphoma cells that express CD47/CD20 endogenously. Further study was conducted through ADCP to investigate biological activity of Q-1801 blocking the binding of macrophage SIRPα to CD47 in Raji cells, thereby synergistically enhancing the CD20 antibody Rituxan dependent macrophage phagocytosis of human Burkitt's lymphoma cell Raji.

Monocytes were separated from peripheral blood mononuclear cells (PBMCs) from two different donors, and were added with 50 ng/mL Human Recombinant M-CSF to induce differentiation into macrophages. Raji cells were labeled with green fluorescent CFSE, and were inoculated into a 96 well plate with macrophages at a ratio of 2:1, and then the plate was added with different concentrations of the CD20 antibody Rituxan alone or in combination with SIRPα antibodies such as Q-1801. After incubating at 37° C. for 2 hours, the reaction was terminated, and then the APC anti-human CD11b antibody was incubated. The percentage of APC/FITC double positive cells at each concentration of antibody was obtained by FACS reading, which was the percentage of macrophages exhibiting phagocytic behavior.

The results are shown in FIG. 48. Donor: P121031405C, Rituxan induced macrophage phagocytosis of Raji cells in a concentration dependent manner, with a maximum phagocytic percentage of approximately 26.57% and an EC50 value of approximately 0.02115 μg/mL; The phagocytic effect of Q-1801 synergizing Rituxan dependent macrophages to phagocytose human Burkitt's lymphoma Raji cells was strong, with a maximum phagocytic percentage increased from 26.57% to 32.38%, and an EC50 value of approximately 0.01188 μg/mL. The single component control CHO71 was equivalent to Q-1801, with a maximum phagocytic percentage increased from 26.57% to 32.14%, and an EC50 value of approximately 0.01302 μg/mL. The maximum phagocytic percentage of positive control QP026249 (KWAR23 analog) increased from 26.57% to 30.09%, with an EC50 value of approximately 0.01765 μg/mL. The maximum phagocytic percentage of positive control QP37503751 (1H9 analog) was about 28.07%, and the EC50 value was about 0.01485 μg/mL.

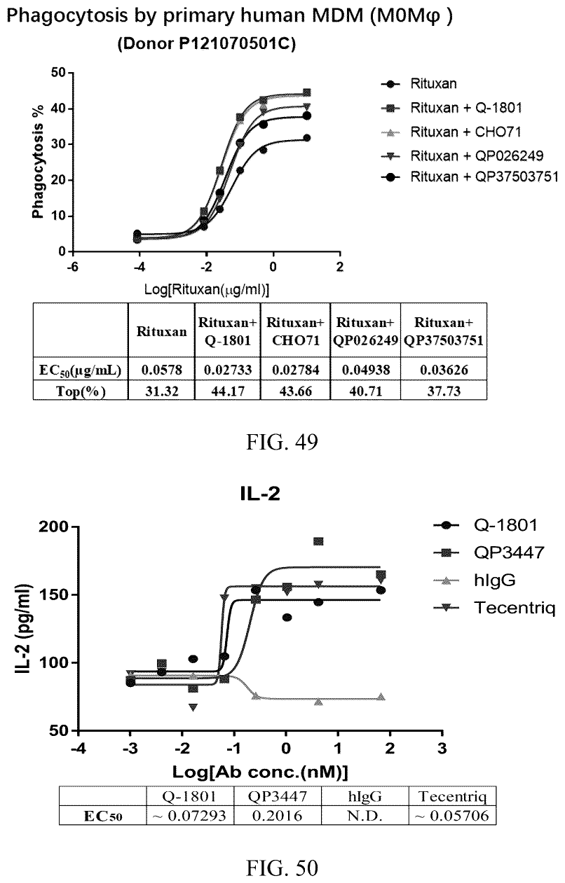

The results are shown in FIG. 49. Donor: P121070501C, Rituxan induced macrophage phagocytosis of Raji cells in a concentration dependent manner, with a maximum phagocytic percentage of approximately 31.32% and an EC50 value of approximately 0.0578 μg/mL; The result of combination of Q-1801+Rituxan showed that Q-1801 significantly increased Rituxan dependent macrophages to phagocytose human Burkitt's lymphoma Raji cells, with a maximum phagocytic percentage of approximately 44.17%, and an EC50 value of approximately 0.02733 g/mL. The maximum phagocytic percentage of the single component control CHO71 was approximately 43.66%, and the EC50 value was approximately 0.01302 μg/mL. The maximum phagocytic percentage of positive control QP026249 (KWAR23 analog) was approximately 40.71%, and the EC50 value was approximately 0.04938 μg/mL. The maximum phagocytic percentage of positive control QP37503751 (1H9 analog) was approximately 37.73%, and the EC50 value was approximately 0.03626 μg/mL.

In summary, the combination of Q-1801 and Rituxan significantly enhances Rituxan dependent macrophage phagocytosis of Raji cells.

### Example 23: Q-1801 can Stimulate T Cell Proliferation in Mixed Lymphocyte Reactions

Mixed lymphocyte reaction refers to the co-culture of human T cells and allogeneic dendritic cells, where lymphocytes undergo activation and proliferation upon stimulation by allogeneic antigens, producing a variety of cytokines. PD-L1 antibodies exhibit concentration dependent immune suppression signals that block the binding of PD-1/PD-L1, stimulating T cell proliferation and releasing cytokines such as IL-2/IFN-T. The IL-2/IFN-T release level was detected through ELISA, to further investigate the biological activity of Q-1801 in stimulating T cell proliferation in vitro in mixed lymphocyte reactions.

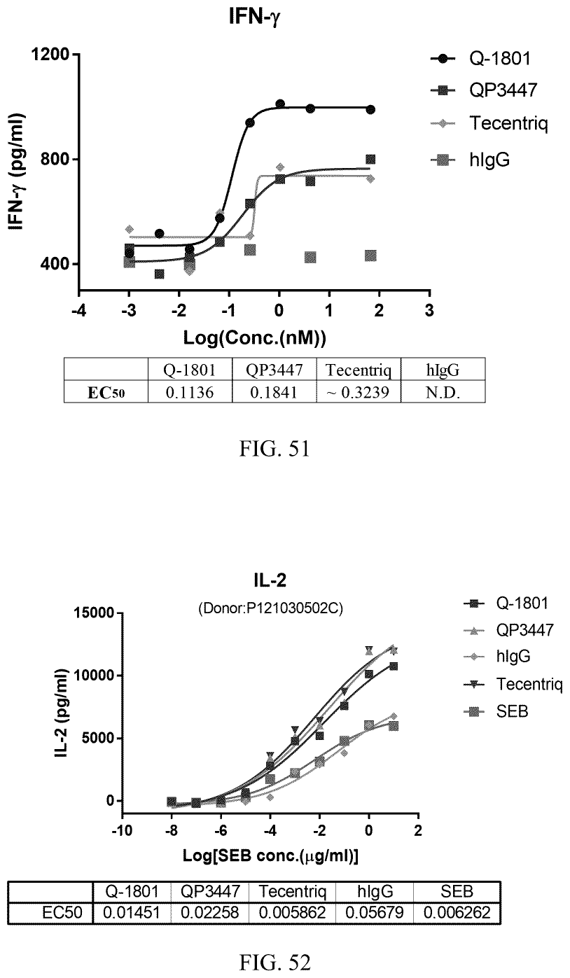

The monocytes were separated from PBMCs, and added with rhGM-CSF and rhIL-4, then induced into dendritic cells (DCs); CD4+T cells were separated from PBMCs of another donor. DC cells and T cells were mixed in a 1:10 ratio, added with antibodies of different concentrations, and subjected to co-culture for 2-5 days. The expression of IL-2 and IFN-γ in the culture supernatant was measured. The results showed that Q-1801, single component control QP3447, and control antibody Tecentriq can all stimulate T cell proliferation and enhance the production of IL-2 and IFN-γ in mixed lymphocyte reaction (MLR). (As shown in FIG. 50, FIG. 51).

In summary, Q-1801 can stimulate T cell proliferation and enhance the production of IL-2 and IFN-γ in mixed lymphocyte reaction (MLR), with IFN-γ secretion being superior to Tecentriq.

### Example 24: Biological Activity of Q-1801 Stimulating Human PBMC In Vitro Proliferation

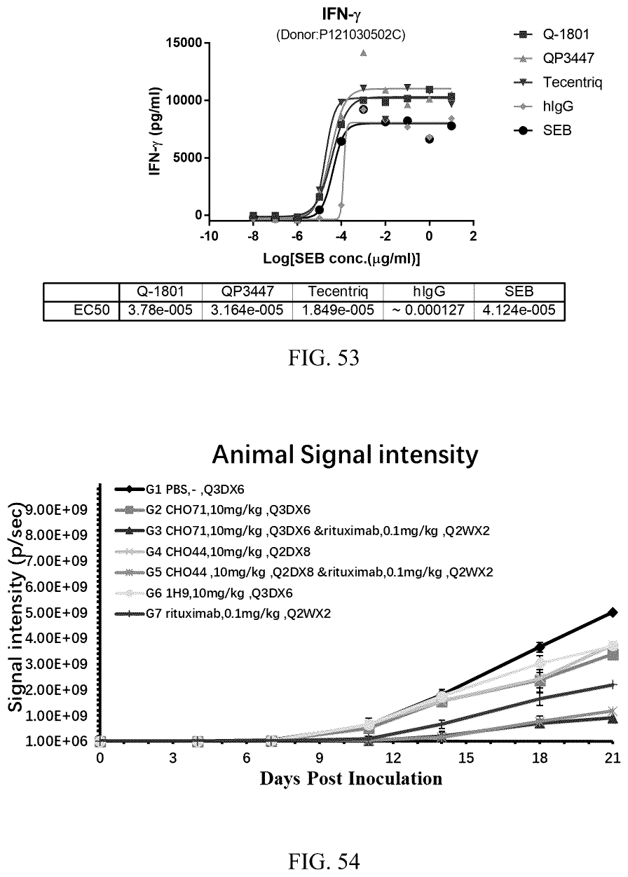

Human peripheral blood mononuclear cells (PBMCs) are composed of various white blood cells, mainly including monocytes, B cells, T cells, NK cells, dendritic cells, and macrophages, etc. Superantigen SEB was added in vitro to stimulates PBMCs, and through the presentation and activation of APC cells, lymphocytes were activated and began to proliferate, producing a variety of cytokines. PD-L1 antibody enhances T cell proliferation and the release of cytokines such as IL-2/IFN-T by blocking the immunosuppressive signal of PD-1/PD-L1 binding. The biological activity of Q-1801 in PBMC in vitro proliferation assay was further investigated by detecting IL-2/IFN-T release through ELISA. PBMC cells were inoculated into a 96 well plate, SEB at different concentrations were prepared and added to the wells containing PBMC cells. Then Q-1801 and other control antibodies were added thereto, mixed gently, and subjected to culture for 2-5 days. IL-2 secretion in cell culture supernatant was detected by ELISA, and IFN-γ secretion in cell culture supernatant was detected by ELISA. The results showed that Q-1801 significantly enhanced the activation and proliferation of PBMCs, as well as the production of IL-2 and IFN-γ, in the in vitro proliferation assay of PBMCs stimulated by SEB. The single component control QP3447 could significantly enhance the activation and proliferation of PBMCs, as well as the production of IL-2 and IFN-γ, in the in vitro proliferation assay of PBMCs stimulated by SEB. The control antibody Tecentriq could significantly enhance the activation and proliferation of PBMCs, as well as the production of IL-2 and IFN-γ, in the in vitro proliferation assay of PBMCs stimulated by SEB. The results are shown in FIG. 52 and FIG. 53.

In summary, Q-1801 can significantly enhance the activation and proliferation of PBMCs, as well as the production of IL-2 and IFN-γ, in the in vitro proliferation assay of PBMCs stimulated by SEB, and the activity is comparable with Tecentriq.

### Example 25: Inhibition of Raji-Luc Tumor Growth by Q-1801 in B-NDG-hSIRPα Mouse Model

B-luc-GFPRaji cells resuspended in PBS were inoculated into female B-NDG hSIRPα mice via tail vein at a concentration of 1×10⁵ cells/0.2 mL, with a volume of 0.2 mL/mouse. On the 0th day after inoculation, the tumor inoculation status was observed using a small animal imaging device. On the 4th day after inoculation, the growth of tumor cells was measured using a small animal imaging device. Tumor bearing mice were eliminated if the imaging signal was too strong or too weak. 70 mice with moderate tumor imaging signals were selected and randomly assigned to 7 groups, with 10 mice in each group. The average imaging signal of each group was about 2.92E+06p/sec. The day of grouping was defined as Day 0 (D0), and on the day of grouping, administration was beginning according to the experimental scheme design, with a administration volume of 10 μL/g. If imaging and administration were scheduled on the same day, then an interval of more than 4 hours was set between imaging and administration. The detailed administration method, dosage, and route are shown in Table 24.

TABLE 24
B-luc-GFP Raji tumor model pharmacological experiment scheme

| | Number | | | | |
|---|---|---|---|---|---|
| | of | | | Mode of | Cycle of |
| Group | animals | Administrated group | Dose (mg/kg) | administration | administration |
| 1 | 10 | Vehicle control (PBS) | — | i.v. | Q3D |
| 2 | 10 | CHO71 | 10 | i.p. | Q3D |
| 3 | 10 | CHO71 + rituximab | 10 + 0.1 | i.p. + i.v | Q3D + Q2W |
| 4 | 10 | CHO44 | 10 | i.p. | Q2D |
| 5 | 10 | CHO44 + rituximab | 10 + 0.1 | i.p. + i.v | Q2D + Q2W |
| 6 | 10 | 1H9 | 10 | i.p. | Q3D |
| 7 | 10 | rituximab | 0.1 | i.v. | Q2W |

After starting the administration, the status of the mice was strictly monitored every ay, an a small animal live imaging device was used to image the mice twice a week, and obtain imaging signal maps and signal intensities. After the last administration, the weight and tumor growth of the experimental animals (detected and recorded by a small animal imaging device) were observed for another 3 days, and then the mice were subjected to euthanasia.

The following analysis method was selected for data analysis: TGI (%)=(1−TR/CR)×100%, where TR and CR are the relative tumor imaging signal sizes (R) of the treatment group and the control group at a specific time point, respectively; R=Vt/V0 (V0 is the mean imaging signal at grouping, Vt is the mean imaging signal at each measurement after treatment).

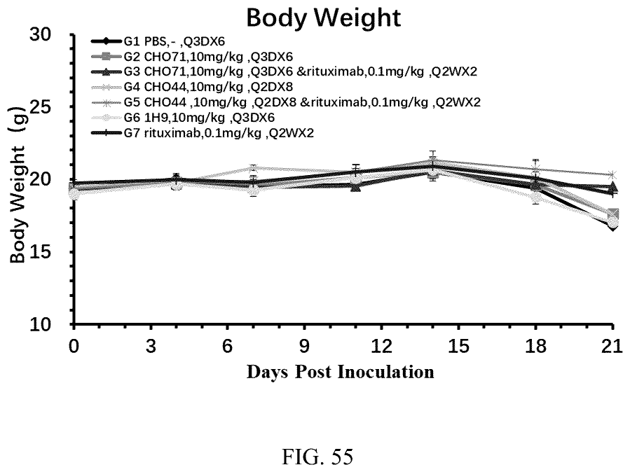

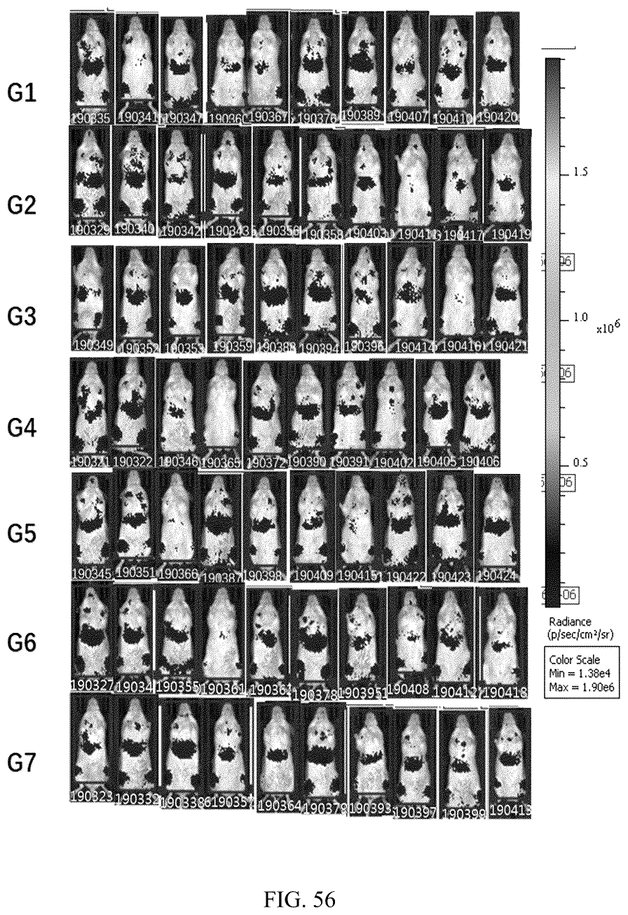

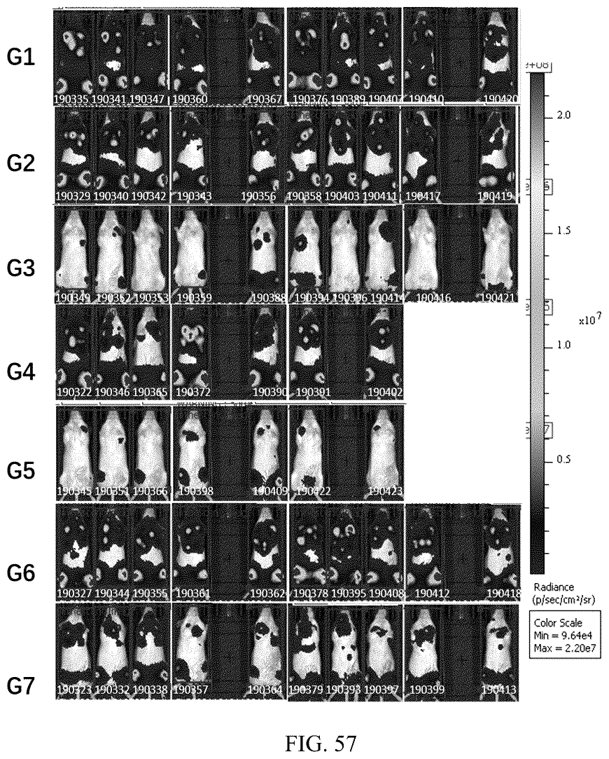

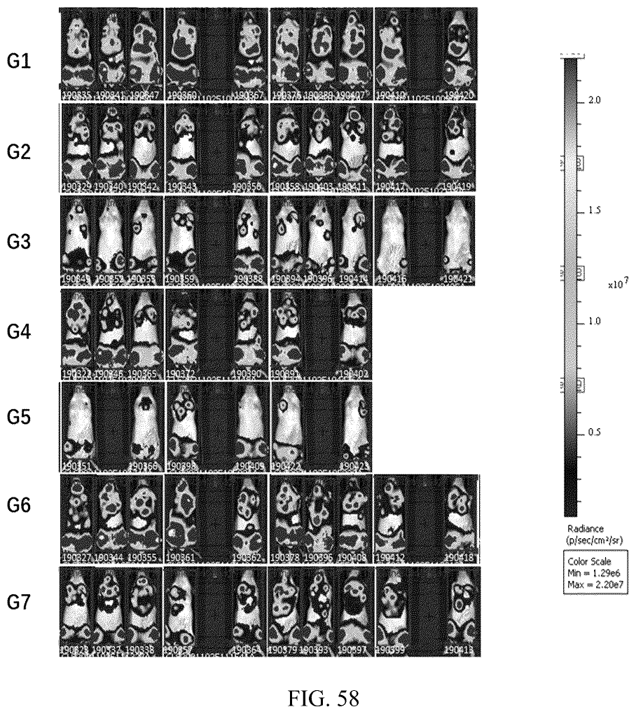

The tumor growth inhibition (TGI) was calculated based on the imaging signal intensity, and the results are shown in Table 25 and FIG. 54. FIG. 54 shows the trend of tumor growth after administration. The body weight of each group of mice and the tumor imaging signal intensity of each individual mouse after administration are shown in FIGS. 55 to 58. FIG. 55 shows the trend of weight changes in animals after administration. FIG. 56 shows the live imaging photos of mice on day 0 after grouping. FIG. 57 shows the live imaging photos of mice on day 7 after grouping. FIG. 58 shows the live imaging photos of mice on day 14 after grouping.

TABLE 25
The effect of the tested examples on the tumor volume of
B-NDG-hSIRPa mice transplanted with B-luc-GFPRaji cells

| | | Tumor imaging signal intensity (p/sec) ^{a} | | | |
|---|---|---|---|---|---|
| | | Before | Day 14 of group | TGI | |
| Group | Tested sample | Administration | administration | (%) | P^{b} |
| G1 | PBS | 2.92E+06 ± 2.78E+05 | 3.65E+09 ± 2.11E+08 | — | — |
| G2 | CHO71 | 2.91E+06 ± 2.89E+05 | 2.37E+09 ± 1.15E+08 | 35.2 | ****<0.0001 |
| G3 | CHO71 + rituximab | 2.90E+06 ± 2.94E+05 | 7.04E+08 ± 1.66E+08 | 80.8 | ****<0.0001 |
| G4 | CHO44 | 2.93E+06 ± 3.27E+05 | 2.43E+09 ± 2.37E+08 | 33.4 | **<0.01 |
| G5 | CHO44 + rituximab | 2.93E+06 ± 3.04E+05 | 7.64E+08 ± 2.19E+08 | 79.1 | ****<0.0001 |
| G6 | 1H9 | 2.91E+06 ± 3.17E+05 | 3.05E+09 ± 1.96E+08 | 16.6 | 0.050 |
| G7 | rituximab | 2.93E+06 ± 3.04E+05 | 1.64E+09 ± 1.67E+08 | 55.2 | ****<0.0001 |

Note:
^{a} Mean ± SEM
^{b}Statistical comparison of tumor imaging signal intensity between the treatment group and the solvent control group on the 14th day of group administration, t-test.
(**P < 0.01,
****P < 0.0001).

### Experimental Results

On the 14th day after group administration, compared with the PBS control group, the experimental group including 1H₉, CHO71, CHO44, and rituximab significantly inhibited the increase in tumor imaging signal intensity. The combination of CHO71 and CHO44 with rituximab showed a more significant inhibitory effect on the increase of tumor imaging signal intensity compared to the single therapy group (P<0.0001 and P<0.0001, respectively). However, there was no significant decrease in the body weight of the mice during the administration process, indicating that the antibody molecules did not have significant toxic side effects on the mice.

### Example 26: Inhibition of MC38-hPD-L1 Tumor Model Growth in C57BL/6-hPD-L1 Mice

Experimental objective: To evaluate the inhibitory activity of anti-human SIRPα antibodies on tumor growth in C57BL/6-hPD-L1 mice through in vivo pharmacological experiments using the MC38-hPD-L1 tumor model.

MC38-hPD-L1 mouse colon cancer cells in logarithmic growth phase were digested, the culture medium was removed, and washed twice with PBS for cell counting. The cells were seeded subcutaneously on the right side of C57BL/6-hPD-L1 mice, with 5×10⁵/100 μL tumor cells per mouse. When the average tumor volume grew to about 50 mm³, the mice were randomly divided into groups of 10 mice each, and the day of grouping was defined as D0. On the day of grouping, the drug was administered according to the experimental scheme, with a volume of 10 μL/g. The detailed administration method, dosage, and route are shown in Table 26.

TABLE 26
MC38-hPD-L1 tumor model pharmacological experiment scheme

| | Numbers of | | | | |
|---|---|---|---|---|---|
| | animals | Administrated | Dose | Mode of | Cycle of |
| Group | (mice) | group | (mg/kg) | administration | administration |
| 1 | 10 | PBS | — | i.p. | Q2D × 10 |
| 2 | 10 | CH044-L | 7.5 | i.p. | Q2D × 10 |
| 3 | 10 | CH044-M | 15 | i.p. | Q2D × 10 |
| 4 | 10 | CH044-H | 30 | i.p. | Q2D × 10 |

After starting administration, the body weight and tumor volume of mice were measured twice a week. The calculation method for tumor volume is: tumor volume (mm³)=1/2×(a×b²) (wherein a represents tumor long diameter and b represents tumor short diameter. The experiment was terminated one week after the last administration, and the mice were euthanized. The tumors were isolated, weighed, and photographed.

The following analysis method was used for data analysis: relative tumor proliferation rate, T/C (%), which is the percentage ratio of relative tumor volume or weight between the treatment group and the control group at a certain time point. The calculation formula is: T/C %=TRTV/CRTV×100% (TRTV: treatment group average RTV; CRTV: control group average RTV; RTV=Vt/V0, where V0 represents the tumor volume of mice at the time of grouping and Vt represents the tumor volume of mice after treatment; The relative tumor growth inhibition rate TGITV (%) was calculated based on tumor volume, using the formula: TGITV %=(1−T/C)×100% (T and C are the relative tumor volume (RTV) of the treatment group and the control group at a specific time point, respectively); The tumor growth inhibition rate TGITW (%) based on changes in tumor weight, using the formula: TGITW %=(1−TWtreat/TWvehicle)×100% (TWtreat and TWvehicle are the average values of tumor weight at the end point of the experiment in the treatment group and control group mice, respectively).

### Experimental Results

The average tumor volume of PBS control group mice on the 19th day after administration was 402.47 mm³. On the 19th day after administration, the average tumor volumes of the antibody molecules groups CHO44-L (7.5 mg/kg), CHO44-M (15 mg/kg), CHO44-H (30 mg/kg), and QP3447 (10 mg/kg) were 198.20 mm³, 144.21 mm³, 92.54 mm³, and 89.33 mm³, respectively. Compared with the control group PBS, CHO44-L (7.5 mg/kg, TGI=58.07%), CHO44-M (15 mg/kg, TGI=73.19%), CHO44-H (30 mg/kg, TGI=87.94%), and QP3447 (10 mg/kg, TGI=88.92%) could significantly inhibit tumor growth (P<0.05*, P<0.05*, P<0.01**, and P<0.01**), and showed a dose gradient dependence.

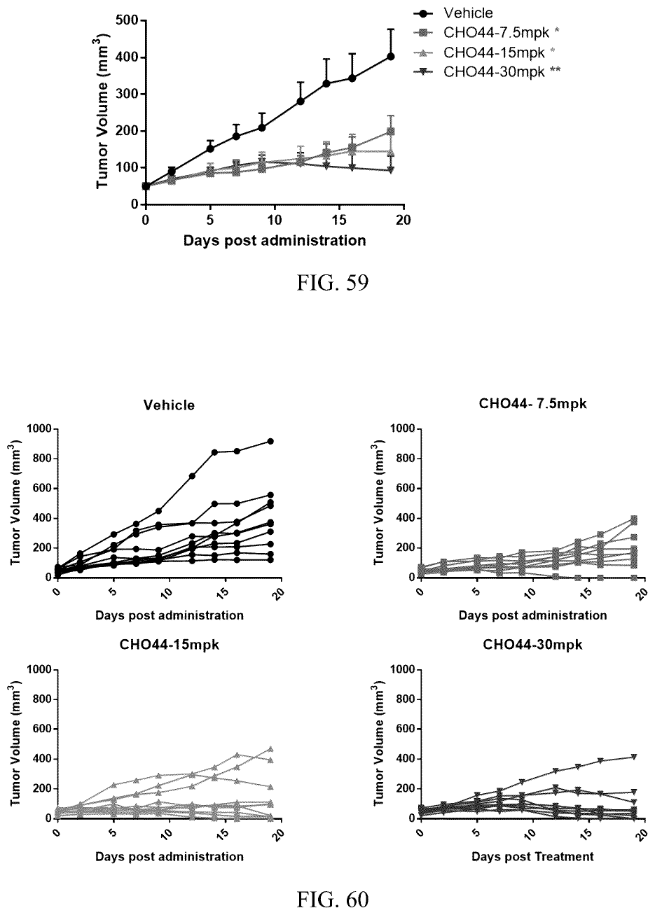

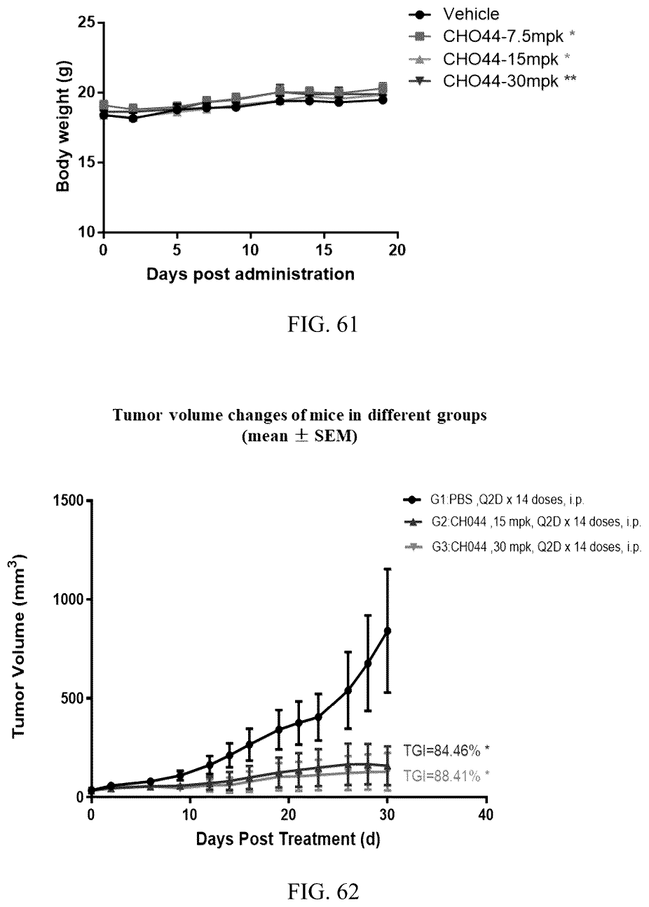

The tumor growth inhibition rate (TGITV) was calculated based on the tumor volume of mice, and the results are shown in Table 27. The pharmacological effects of antibody molecules CHO44-L (7.5 mg/kg), CHO44-M (15 mg/kg), and CHO44-H (30 mg/kg) on the MC38-hPD-L1 tumor model are shown in FIG. 59; FIG. 60 shows the tumor growth curves of mice in the PBS group, CHO44-L (7.5 mg/kg), CHO44-M (15 mg/kg), and CHO44-H (30 mg/kg) administrated groups after group administration; FIG. 61 shows the weight change curves of mice of MC38-hPD-L1 colon cancer tumor model in each group after administration.

TABLE 27
The effect of the tested samples on the tumor volume of
C57BL/6-hPD-L1 mice transplanted with MC38-hPD-L1 cells

| | | Tumor volume (mm³) ^{a} | | | |
|---|---|---|---|---|---|
| | | D0 when | D19 after group | | |
| Group | Tested sample | grouping | administration | TGI_{TV}(%) | P^{b} |
| G1 | PBS | 49.74 | 402.47 | — | — |
| G2 | CHO44-L (7.5 mg/kg) | 50.31 | 198.20 | 58.07 | 0.0330* |
| G3 | CHO44-M (15 mg/kg) | 49.62 | 144.21 | 73.19 | 0.0105* |
| G4 | CHO44-H (30 mg/kg) | 50.01 | 92.54 | 87.94 | 0.0016** |

Note:
^{a} The data is represented by mean.
^{b}Compared with G1 group, independent sample T-test was used,
*P < 0.05;
**P < 0.01.

Through the in vivo pharmacological test of MC38-hPD-L1 tumor model in C57BL/6-hPD-L1 mice, we found that antibody molecules CHO44 and QP3447 could significantly inhibit tumor growth. The inhibitory effect of CHO44 on tumor growth was dose-dependent, and there was no significant decrease in mouse body weight during administration, indicating that antibody molecules had no significant toxic side effects on mice.

### Example 27: Inhibition of CT26-hPD-L1&hCD47 Tumor Model Growth in BALB/c-hPD-1&hSIRPα Mice

Experimental objective: To evaluate the inhibitory activity of anti-human SIRPα antibodies on tumor growth in BALB/c-hPD-1&hSIRPα mice through in vivo pharmacological experiments using the CT26-hPD-L1&hCD47 tumor model.

Experimental steps: CT26-hPD-L1&hCD47 mouse colon cancer cells in logarithmic growth phase were digested, the culture medium was removed, and washed twice with PBS for cell counting. The cells were seeded subcutaneously on the right side of BALB/c-hPD-1&hSIRPα mice, with 1.5×10⁶/100 μL tumor cells per mouse. When the average tumor volume grew to about 40 mm³, the mice were randomly divided into groups of 6 mice each, and the day of grouping was defined as D0. On the day of grouping, the drug was administered according to the experimental scheme, with a volume of 10 μL/g. The detailed administration method, dosage, and route are shown in Table 28.

TABLE 28
CT26-hPD-L1 tumor model pharmacological experiment scheme

| | Numbers of | | | | |
|---|---|---|---|---|---|
| | animals | Administrated | Dose | Mode of | Cycle of |
| Group | (mice) | group | (mg/kg) | administration | administration |
| 1 | 6 | PBS | — | i.p. | Q2D × 14 |
| 2 | 6 | CH044-L | 15 | i.p. | Q2D × 14 |
| 3 | 6 | CH044-H | 30 | i.p. | Q2D × 14 |

After starting administration, the body weight and tumor volume of mice were measured twice a week. The calculation method for tumor volume is: tumor volume (mm³)=1/2×(a×b²) (wherein a represents tumor long diameter and b represents tumor short diameter. The experiment was terminated one week after the last administration, and the mice were euthanized. The tumors were isolated, weighed, and photographed.

The following analysis method was used for data analysis: relative tumor proliferation rate, T/C (%), which is the percentage ratio of relative tumor volume or weight between the treatment group and the control group at a certain time point. The calculation formula is: T/C %=TRTV/CRTV×100% (TRTV: treatment group average RTV; CRTV: control group average RTV; RTV=Vt/V0, where V0 represents the tumor volume of mice at the time of grouping and Vt represents the tumor volume of mice after treatment; The relative tumor growth inhibition rate TGI_{TV} (%) was calculated based on tumor volume, using the formula: TGI_{TV} %=(1−T/C)×100% (T and C are the relative tumor volume (RTV) of the treatment group and the control group at a specific time point, respectively); The tumor growth inhibition rate TGI_{TW} (%) based on changes in tumor weight, using the formula: TGI_{TW} %=(1−TWₜᵣₑₐₜ/TW_{vehicle})×100% (TWₜᵣₑₐₜ and TW_{vehicle} are the average values of tumor weight at the end point of the experiment in the treatment group and control group mice, respectively).

### Experimental Results

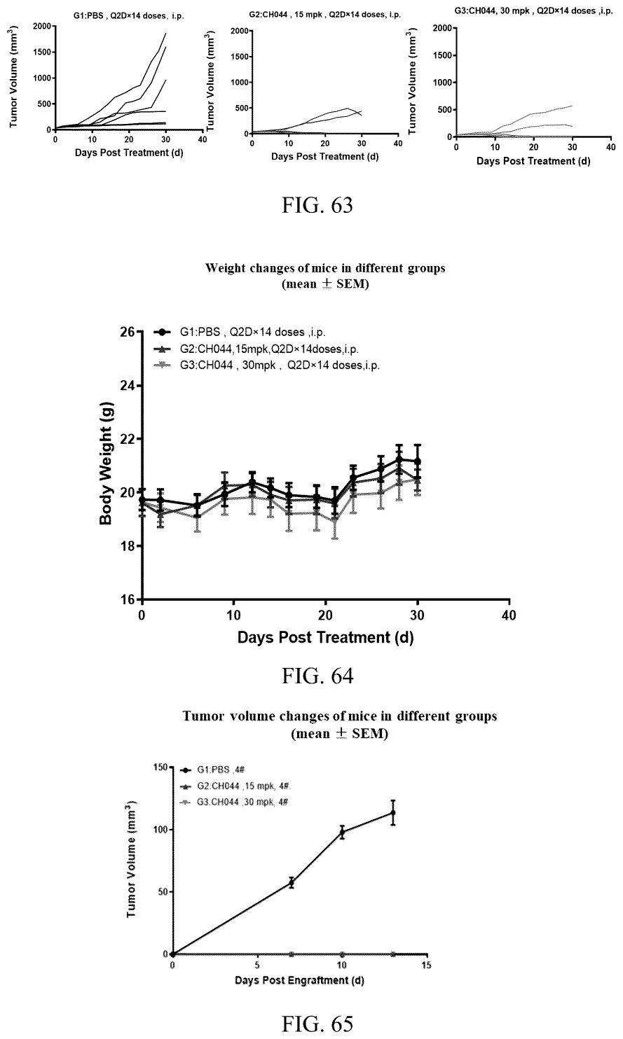

The average tumor volume of PBS control group mice on the 30th day after administration was 841.5 mm³. On the 30th day after administration, the average tumor volumes of the CHO44-L (15 mg/kg) and CHO44-H (30 mg/kg) antibody groups were 158.3 mm³ and 128.1 mm³, respectively. The tumor growth inhibition rate (TGI_{TV}) was calculated based on the mouse tumor volume. Compared with the control group PBS, CHO44-L (15 mg/kg, TGI=80.85%) and CHO44-H (30 mg/kg, TGI=83.9%) significantly inhibited tumor growth (P<0.05*, P<0.05*, P<0.01**, and P<0.01**), as shown in Table 29 and FIG. 62. FIG. 63 shows the tumor growth curves of each mouse in the PBS group, CHO44-L (15 mg/kg), and CHO44-H (30 mg/kg) administrated groups after group administration, wherein three mice in the CHO-L group had tumor elimination on day D14 (2 mice) and day D23 (1 mouse), while four mice in the CHO44-H group had tumor elimination on day D12 (2 mice), day D14 (1 mouse), and day D21 (1 mouse). FIG. 64 shows the weight change curves of mice in each group of CT26-hPD-L1&hCD47 colon cancer tumor model after administration.

TABLE 29
The effect of the tested samples on the tumor volume of B-
hPD1&hSIRPα mice transplanted with CT26-hPD-L1&hCD47 cells

| | | Tumor volume (mm³) ^{a} | | | |
|---|---|---|---|---|---|
| | | D0 when | D19 after group | | |
| Group | Tested sample | grouping | administration | TGI_{TV}(%) | P_{b} |
| G1 | PBS | 34.61 | 841.50 | — | — |
| G2 | CHO44-L (15 mg/kg) | 34.68 | 158.30 | 84.66 | 0.039* |
| G3 | CHO44-H (30 mg/kg) | 34.59 | 128.10 | 88.41 | 0.030* |

In the pharmacological evaluation of BALB/c-hPD1/hSIRPα mice subcutaneously inoculated with CT26-hPDL1 hCD47 model, mice with tumor elimination were inoculated with CT26-hPDL1&hCD47 to evaluate tumor growth.

The treatment of cells, measurement of tumor volume, and calculation of TGI in the experiment were all based on the methods used for in vivo efficacy evaluation during the initial inoculation. The number of cell inoculation was consistent with the first inoculation quantity, and the inoculation position was on the left side, opposite to the first inoculation position. The tumor volume was measured twice a week.

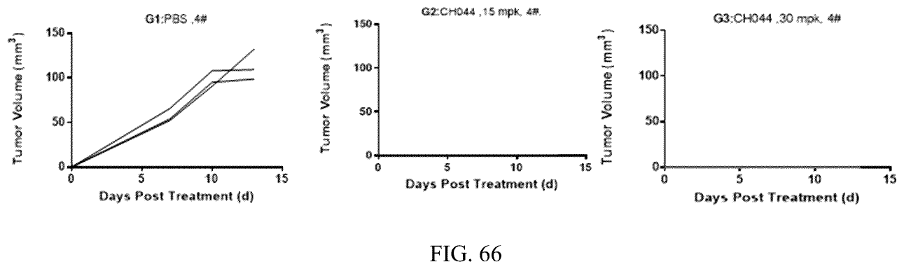

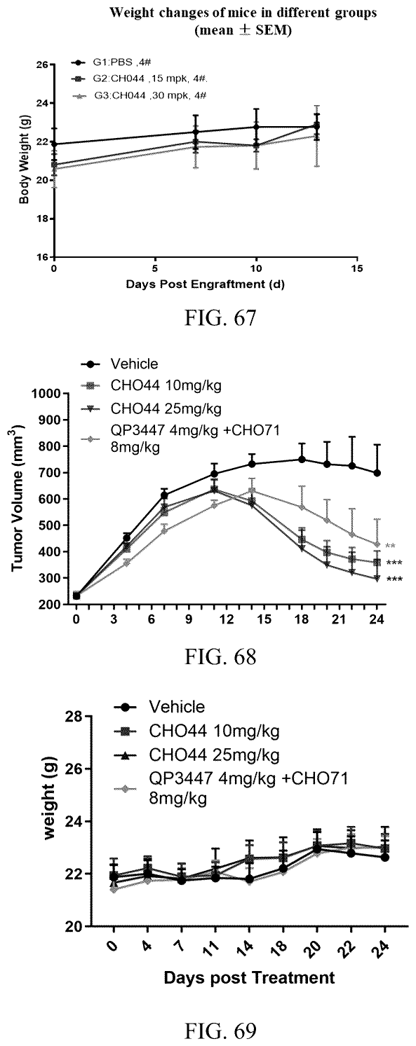

It was found that after re-inoculation of cells, the average tumor volume in the PBS group grew to 113.56 mm³ on the 14th day after inoculation. On the 14th day after re-inoculation of antibody molecules CHO44-L (15 mg/kg) and CHO44-H (30 mg/kg) groups, the tumors did not grow. The results are shown in FIG. 65, and the growth curves and body weights of each group of mice are shown in FIGS. 66 and 67. The results indicate that mice developed immune memory during the initial treatment, and when mice with tumor elimination were inoculated again, the tumor did not grow.

### Example 28: Pharmacodynamic Evaluation in Female NCG Mouse PBMC Reconstruction Model of Subcutaneous Transplantation of Non-Small Cell Lung Cancer HCC827 Cell Line

Human non-small cell lung cancer HCC827 cells were subcutaneously inoculated into female NCG mice at a concentration of 3.0E+06 cells/100 μl. When the average volume of the tumor reaches 100 mm³, it was defined as Day D0. On Day D0, 5.5E+06 PBMCs were inoculated via ip. After 7 days (D7), the mice were randomly divided into 4 groups based on tumor volume, with 8 mice in each group: G1/PBS, G2/CHO44-10 mg/kg, G3/CHO44-25 mg/kg, G4/CHO71-8 mg/kg+QP3447-4 mg/kg. After starting administration, the body weight of mice was measured twice a week, and the tumor volume was measured 2-3 times a week. According to the statistical analysis of tumor volume data on D24 day, compared with the control group PBS, the G2/CHO44-10 mg/kg, G3/CHO44-25 mg/kg, G4/CHO71-8 mg/kg+QP3447-4 mg/kg groups all showed significant inhibitory activity on tumor growth, and CHO44 exhibited dose gradient dependence. TGI were G2: 72.98% (p<0.0001), G3: 86.28% (p<0.0001), G4: 58.02% (p=0.0023), respectively (FIG. 68, Table 30). The body weight changes during administration are shown in FIG. 69.

TABLE 30
Changes in tumor volume inhibition rate (TGITV) after administration in different groups of mice

| TGITV | | | | | | | | | | | |
|---|---|---|---|---|---|---|---|---|---|---|---|
| | Tested | | | | | | | | | | P value |
| Group | drug | 0 | 4 | 7 | 11 | 14 | 18 | 20 | 22 | 24 | (D 24) |
| G2 | CHO44 | 0.00% | 19.32% | 17.61% | 12.85% | 28.15% | 58.67% | 67.02% | 71.78% | 72.98% | <0.0001 |
| | (10 mg/kg) | | | | | | | | | | |
| G3 | CHO44 | 0.00% | 15.31% | 12.25% | 14.15% | 31.60% | 65.70% | 76.76% | 82.22% | 86.28% | <0.0001 |
| | (25 mg/kg) | | | | | | | | | | |
| G4 | QP3447 | 0.00% | 44.06% | 35.86% | 26.06% | 20.36% | 35.06% | 42.59% | 52.76% | 58.02% | 0.0023 |
| | 4 mg/kg + | | | | | | | | | | |
| | CHO71 | | | | | | | | | | |
| | 8 mg/kg | | | | | | | | | | |

Although the content of the present invention has been described in detail through the preferred embodiments mentioned above, it should be recognized that the above description should not be considered a limitation of the present invention. After reading the above disclosure, it will be apparent to those skilled in this art that there are various modifications and substitutions to the present invention. Therefore, the scope of protection of the present invention should be limited by the appended claims.

1. A bispecific antibody targeting SIRPα and PD-L1 or antigen binding fragment thereof, comprising: a SIRPα binding domain and a PD-L1 binding domain; wherein,
the SIRPα binding domain comprises: a heavy chain variable region and a light chain variable region; the heavy chain variable region comprises: VHCDR1, VHCDR2, and VHCDR3 with amino acid sequences as shown in SEQ ID NOs: 3, 4, and 5, respectively; the light chain variable region comprises: VLCDR1, VLCDR2, and VLCDR3 with amino acid sequences as shown in SEQ ID NOs: 37, 38, and 9, respectively;
the PD-L1 binding domain comprises: a VHH fragment, which comprises CDR1, CDR2, and CDR3 with amino acid sequences as shown in SEQ ID NOs: 63, 64, and 65, respectively.

2. The bispecific antibody targeting SIRPα and PD-L1 or antigen binding fragment thereof according to claim 1, wherein the sequence of the heavy chain variable region of the SIRPα binding domain is as shown in SEQ ID NO: 17 or has at least 85% sequence identity with SEQ ID NO: 17; or the sequence of the light chain variable region of the SIRPα binding domain is selected from SEQ ID NO: 18 or has at least 85% sequence identity with SEQ ID NO: 18.

3. The bispecific antibody targeting SIRPα and PD-L1 or antigen binding fragment thereof according to claim 1, wherein the sequence of the VHH fragment is as shown in SEQ ID NO: 62 or has at least 85% sequence identity with SEQ ID NO: 62.

4. The bispecific antibody targeting SIRPα and PD-L1 or antigen binding fragment thereof according to claim 1, wherein the bispecific antibody or antigen binding fragment thereof further comprises: a heavy chain constant region selected from human-derived IgG1, IgG2, IgG3, or IgG4 or variants thereof; and a light chain constant region selected from human-derived κ, λ chains or variants thereof.

5. The bispecific antibody targeting SIRPα and PD-L1 or antigen binding fragment thereof according to claim 4, wherein the heavy chain constant region comprises: an Fc fragment or variants thereof; the variant of the Fc fragment is derived from IgG1, according to EU Numbering, including mutation sites: L234A, L235A, and K338A.

6. The bispecific antibody targeting SIRPα and PD-L1 or antigen binding fragment thereof according to claim 4, wherein the bispecific antibody or antigen binding fragment thereof comprises: a first polypeptide chain and a second polypeptide chain;
the first polypeptide chain comprises: the heavy chain variable region of the SIRPα binding domain, the heavy chain constant region, and the VHH fragment; the VHH fragment is fused with the N-terminus of the heavy chain variable region of the SIRPα binding domain, or the VHH fragment is fused with the C-terminus of the heavy chain constant region;
the second polypeptide chain comprises: the light chain variable region of the SIRPα binding domain and the light chain constant region; alternatively, the first polypeptide chain comprises: the heavy chain variable region of the SIRPα binding domain and the heavy chain constant region,
the second polypeptide chain comprises: the light chain variable region of the SIRPα binding domain, the light chain constant region, and the VHH fragment; the VHH fragment is fused with the N-terminus of the light chain variable region of the SIRPα binding domain.

7. (canceled)

8. The bispecific antibody targeting SIRPα and PD-L1 or antigen binding fragment thereof according to claim 6, wherein the bispecific antibody or antigen binding fragment thereof is a symmetrical structure comprising two first polypeptide chains and two second polypeptide chains.

9. The bispecific antibody targeting SIRPα and PD-L1 or antigen binding fragment thereof according to claim 6, wherein the bispecific antibody or antigen binding fragment thereof further comprises: a linking sequence; preferably, the linking sequence may be selected from (GGGGS)n, wherein n is an integer from 1 to 4.

10. The bispecific antibody targeting SIRPα and PD-L1 or antigen binding fragment thereof according to claim 6, wherein the amino acid sequence of the first polypeptide chain is as shown in any one of SEQ ID NOs: 66, 26, 69, 84, 85; or the amino acid sequence of the second polypeptide chain is as shown in any one of SEQ ID NOs: 67, 68, 82, 83.

11. The bispecific antibody targeting SIRPα and PD-L1 or antigen binding fragment thereof according to claim 10, wherein the amino acid sequence of the first polypeptide chain is as shown in SEQ ID NO: 66, and the amino acid sequence of the second polypeptide chain is as shown in SEQ ID NO: 67.

12. A drug comprising the bispecific antibody targeting SIRPα and PD-L1 or antigen binding fragment thereof according to claim 1.

13. The drug according to claim 12, wherein the drug further comprises one or more other cancer therapeutic agents.

14. A nucleic acid molecule encoding the bispecific antibody targeting SIRPα and PD-L1 or antigen binding fragment thereof according to claim 1.

15. A vector comprising the nucleic acid molecule according to claim 14.

16. A host cell transformed with the vector according to claim 15.

17. A method for inhibiting or treating a disease, disorder or condition, which comprises a step of administrating the bispecific antibody targeting SIRPα and PD-L1 or antigen binding fragment thereof according to claim 1 to a subject in need thereof.

18. The method according to claim 17, wherein the disease, disorder or condition includes: cancer, solid tumor, chronic infection, inflammatory disease, multiple sclerosis, autoimmune disease, neurological disease, brain injury, nerve injury, polycythemia, hemochromatosis, trauma, septic shock, fibrosis, atherosclerosis, obesity, type II diabetes, allograft dysfunction or arthritis.

19. The method according to claim 18, wherein the cancer is selected from anal cancer, appendiceal cancer, astrocytoma, basal cell cancer, gallbladder cancer, gastric cancer, lung cancer, bronchial cancer, bone cancer, hepatobiliary cancer, pancreatic cancer, breast cancer, liver cancer, ovarian cancer, testicular cancer, renal cancer, renal pelvis and ureter cancer, salivary gland cancer, small intestine cancer, urethra cancer, bladder cancer, head and neck cancer, spinal cancer, brain cancer, cervical cancer, uterine cancer, endometrial cancer, colon cancer, colorectal cancer, rectal cancer, esophageal cancer, gastrointestinal cancer, skin cancer, prostate cancer, pituitary cancer, vaginal cancer, thyroid cancer, laryngeal cancer, glioblastoma, melanoma, myelodysplastic syndrome, sarcoma, teratoma, chronic lymphoblastic leukemia (CLL), chronic myeloid leukemia (CML), acute lymphoblastic leukemia (ALL), acute myeloid leukemia (AML), Hodgkin's lymphoma, non-Hodgkin's lymphoma, multiple myeloma, T or B-cell lymphoma, gastrointestinal stromal tumor, soft tissue tumor, hepatocellular carcinoma or adenocarcinoma.

20. The method according to claim 17, wherein the bispecific antibody targeting SIRPα and PD-L1 or antigen binding fragment thereof is administrated to the subject in combination with one or more other drugs.

21. The method according to claim 20, wherein the other drugs include rituximab.
